(19) United States
(12) Patent Application Publication
(10) Pub. No.: US 2024/0190913 A1
(43) Pub. Date: Jun. 13, 2024

(54) SEPARATION MEDIA AND PURIFICATION METHODS FOR CARBOHYDRATE CONTAINING MOLECULES USING THE SAME

(71) Applicant: Donaldson Company, Inc., Bloomington, MN (US)

(72) Inventors: Jinxiang Zhou, Greenville, SC (US); Graham Temples, Greenville, SC (US); Daniel Henn, Wilmington, DE (US)

(21) Appl. No.: 18/383,468

(22) Filed: Oct. 24, 2023

Related U.S. Application Data
(60) Provisional application No. 63/419,241, filed on Oct. 25, 2022.

(51) Int. Cl. C07K 1/22; B01D 15/38; B01J 20/28; B01J 20/289; B01J 20/32; C07K 14/47

(52) U.S. Cl. CPC C07K 1/22; B01D 15/3809; B01D 15/3828; B01J 20/28038; B01J 20/289; B01J 20/3242; C07K 14/473; B01D 2015/3838

(57) ABSTRACT

Methods of using a separation media to isolate a target molecule that includes a carbohydrate are disclosed. The separation media includes a support substrate and a plurality of separation ligands immobilized on the support substrate. The plurality of separation ligands include an affinity capable of recognizing and binding to a carbohydrate.

### CROSS-REFERENCE TO RELATED APPLICATION

This application claims the benefit of U.S. Provisional Patent Application No. 63/419,241, filed Oct. 25, 2022, which is incorporated herein by reference in its entirety.

### FIELD

The present disclosure relates to separation media and separation devices containing the same. The separation media of the present disclosure may be useful for isolation and/or concentration of biomolecules (e.g., proteins or fragments thereof) that include a carbohydrate.

The separation media of the present disclosure may be used for separations in membrane chromatography. The present disclosure further relates to methods of making and using the separation media.

### INTRODUCTION

Antibody-based therapy is a rapidly advancing field. The ability to quickly obtain high purity and high yields of antibodies is, however, hampered by currently available purification media, methods, and systems. As antibody-based therapies are developed and their production upscaled, there is a need for improved separation media and purification methods.

### SUMMARY

This disclosure describes, in one aspect, a method of isolating a target molecule from an isolation solution. The isolation solution includes an isolation solvent and the target molecule.

The target molecule includes a free protein and the free protein includes a carbohydrate. The method includes contacting the isolation solution with a separation media. The separation media includes a support substrate and a plenarily of separation ligands of the formula SL1 or SL2 immobilized on the support substrate.

In formula SL1 and SL2, Rp¹, Rp³, and Rp⁴ each independently comprise the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, Rp^{L}, Rp^{M} or an isomer thereof:

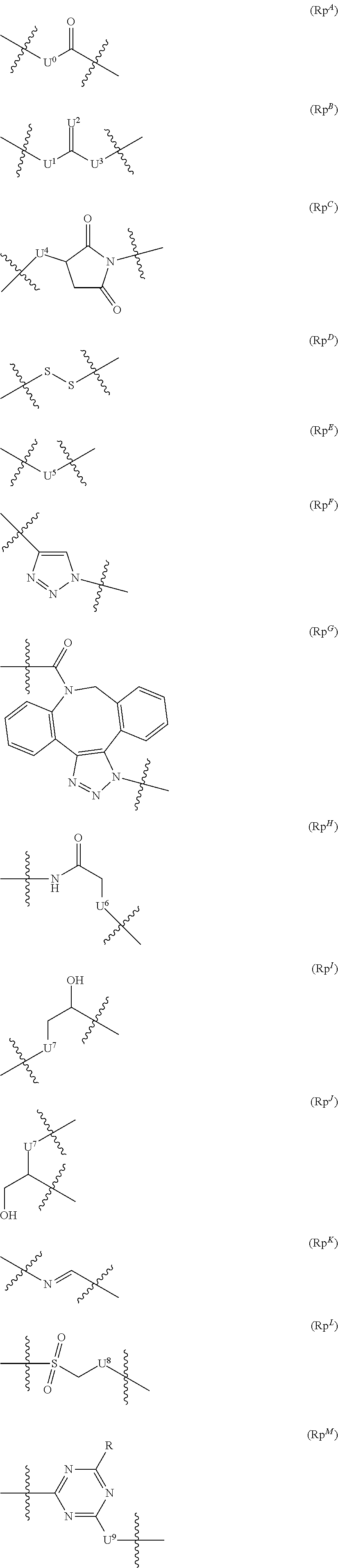

where U⁰, U¹, U², U³, U⁴, U⁵, U⁶, U⁷, U⁸ and U⁹ are each independently NH, N, O, or S and Sp is a spacer. R is an organic group, H, or halogen.

In some embodiments, the target molecule is an antibody or a fragment thereof.

In some embodiments, the target molecule is a glycosylated free protein.

In some embodiments, the target molecule is fetuin.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF FIGURES

FIG. 1A is a flow diagram of a first method for making the separation media of the present disclosure.

FIG. 1B is a flow diagram of a second method for making the separation media of the present disclosure.

FIG. 2A is a flow diagram of a third method for making the separation media of the present disclosure.

FIG. 2B is a flow diagram of a fourth method for making the separation media of the present disclosure.

FIG. 3A is a flow diagram of a fifth method for making the separation media of the present disclosure.

FIG. 3B is a flow diagram of a sixth method for making the separation media of the present disclosure.

FIG. 4A is a schematic of a separation media consistent with embodiments of the present disclosure.

FIG. 4B is a schematic representation of a separation device consistent with embodiments of the present disclosure.

FIG. 5 is a flow diagram of a method of using the separation media and/or separation devices of the present disclosure.

FIG. 6 is a first schematic synthetic strategy for the making of separation media consistent with the present disclosure. This strategy includes the deposition of a polymer onto the support substrate through the grafting on technique. This strategy also includes indirect immobilization of the separation ligands onto the support substrate and an amine assisted coupling method.

FIG. 7 is a second schematic synthetic strategy for the making of separation media consistent with the present disclosure. This strategy includes the deposition of a polymer onto the support substrate through the grafting from technique. This strategy also includes indirect immobilization of the separation ligands onto the support substrate and an amine assisted coupling method.

FIG. 8 is a third schematic synthetic strategy for the making of separation media consistent with the present disclosure. This strategy includes direct immobilization of the separation ligands onto the support substrate and an amine assisted conjugation method.

FIG. 9 is a fourth schematic synthetic strategy for the making of separation media consistent with the present disclosure. This strategy includes direct immobilization of the separation ligands onto the support substrate and an organic solvent assistance conjugation method.

FIGS. 10A and 10B are chromatograms resulting from exposing a separation media having a lectin on the surface to the targets fetuin (FIG. 10A) and asialofetuin (FIG. 10B).

FIG. 11 is a plot showing the relative elution content (%) of fetuin or asialofetuin when elution mixtures of varying NaCl concentrations where employed for elution.

FIG. 12 is a plot showing the static binding capacity of separation media having either lectin having a higher affinity to 2,3 sialic acid than to 2,6 sialic acid or a lectin having a higher affinity to 2,6 sialic acid than to 2,3 sialic acid immobilized on the surface with various targets.

FIG. 13 is a plot showing the porcine thyroglobulin (PTG) static binding capacity (SBC) for a variety of separation media that include a Concanavalin A (Con A) affinity group and prepared by the direct immobilization of the separation ligands and the amine assisted method.

FIG. 14 is a plot showing the porcine thyroglobulin (PTG) SBCs for separation media prepared by direct immobilization of the separation ligands on the support substrate using aqueous buffer conjugation (25 milligrams per milliliter (mg/mL), carb (carbonate), 0.2 micrometer (μm) pore size); direct immobilization of the separation ligands on the support substrate using a salt out conjugation method (25 mg/mL, phos (phosphate), 0.2 μm pore size); direct immobilization of the separation ligands on the support substrate using an organic solvent assisted conjugation method (5 mg/mL, solvent-assisted, 0.2 μm pore size; 5 mg/mL, solvent-assisted, 0.45 μm pore size; 5 mg/mL, solvent-assisted, 1.0 μm pore size); indirect immobilization of the separation ligands on the support substrate using an organic solvent assisted conjugation method (5 mg/mL, solvent-assisted, 0.45 μm pore size, poly-NHS; 5 mg/mL, solvent-assisted, 1 μm pore size, Poly-NHS); and indirect immobilization of the separation ligands on the support membrane (support substrate) using aqueous buffer conjugation (25 mg/mL carb (carbonate), 1 μm pore size Poly-epoxide).

FIG. 15 is a schematic synthetic strategy for depositing a polymer functionalized layer on the support substrate through a grafting from the support substrate.

FIG. 16 is a plot showing the dynamic lentiviral vector bind-and-elute test results using separation media having a dynamic bed volume of 0.025 mL, Con A affinity groups, and pore size of 0.45 μm or 1.0 μm.

FIG. 17 is a plot showing the dynamic lentiviral vector bind-and-elute test results using separation media having a dynamic bed volume of 0.1 mL, Con A affinity groups, and a pore size of 0.45 μm or 1.0 μm. PUREXA-DMAE (weak strong anion-exchange media), and PUREXA-MQ (multimodal anion-exchange media) were also tested.

FIG. 18 is a plot showing the static lentiviral vector bind-and-elute test results using 0.014 mL columns for several separation media that include various affinity groups (Con A, MPBA, and TSA), immobilization methods (direct (Con A) and indirect (Poly-MPBA and Poly-TSA), and pore sizes (0.45 μm or 1.0 μm).

FIG. 19A is a plot showing the static lentiviral vector bind-and-elute test results using 0.014 mL columns and separation media having various affinity groups (GNL, BanLec, MphOH, Gallic Acid, EGCG, serotonin, red (reactive red 120), yellow (cibracon brilliant yellow 3G-P), and blue (reactive blue 4)) immobilized directly or indirectly (Poly-prefix) on the support substrate.

FIG. 19B is a plot showing the dynamic lentiviral vector bind-and-elute test results using 0.025 mL or 0.055 mL columns and separation media having various affinity groups (GNL, BanLec, MphOH, Gallic Acid, EGCG, serotonin, red (reactive red 120), yellow (cibracon brilliant yellow 3G-P), and blue (reactive blue 4)) immobilized directly or indirectly (Poly-prefix) on the support substrate.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used herein, the symbol

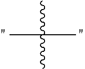

(hereinafter can be referred to as “a point of attachment bond”) denotes a bond that is a point of attachment between two chemical entities, or a chemical entity and a support substrate, one of which is depicted as being attached to the point of attachment bond and the other of which is not depicted as being attached to the point of attachment bond. For example,

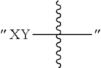

indicates that the chemical entity “XY” is bonded to another chemical entity or support substrate via the point of attachment bond.

The term “organic group” refers to a group that has carbon-hydrogen bonds. The group may also include heteroatoms such as O, S, N, or P. One or more heteroatoms may be catenated at any location in the organic group (e.g., ether, thioether, or amine). A heteroatom may be covalently bonded to a carbon atom through a double bond (e.g., ketone, imine). A heteroatom covalently bonded to a carbon atom may also be covalently bonded to another heteroatom (e.g., phosphodiester, sulfone). One or more functional groups may be included in an organic group, for example, alkane (branched, linear, or cyclic), alkene (branched or linear), alkyne (branched or linear), aromatic, amine (primary, secondary, tertiary, or quaternary), amino, amide, alcohol (primary, secondary, or tertiary), alkoxy, aldehyde, carboxylic acid, ether, ester, imine, phosphoester, phosphodiester, sulfone, sulfonamide, urea, thiourea, thioether, or any combination thereof, and ionized versions thereof. Generally, the organic group may be covalently bonded to compound. The point of attachment of the organic group to the compound may be described in several ways. For example, in some embodiments, the organic group may be described as the monovalent or radical of the respective functional group (e.g., alkyl for alkane, aryl for aromatic ring, aminyl for a primary or secondary amine). In some embodiments, where a general formula is shown with a covalent bond connecting the organic group to a compound, the organic group may be described as the common functional group. For example, if the organic group R is described relative to the formula CH₃CH₂CH₂—R, the organic group may be described, for example, as an aromatic ring.

The term “catenated” in the context of heteroatoms refers to a heteroatom (e.g., O, S, N, P) that replaces at least one carbon atom in a carbon chain. For example, ether groups contain one catenary oxygen atom with at least one carbon atom on each side of the catenary oxygen atom and polyether groups contain more than one catenary oxygen atom with carbon atoms on each side of the more than one catenary oxygen atoms.

The term “aryl” refers to a monovalent group that is aromatic. The aryl group may be carbocyclic or include one or more heteroatoms such as S, N, or O. Example aryl groups include, but are not limited to, phenyl, thiophenyl, furanyl, pyridinyl, pyrimidinyl, piperidinyl, and pyrrolyl.

The term “alkylene” refers to a divalent group that is a radical of an alkane and includes groups that are linear, branched, cyclic, bicyclic, or a combination thereof. Unless otherwise indicated, the alkylene group typically has 1 to 30 carbon atoms. In some embodiments, the alkylene group has 1 to 20 carbon atoms, 1 to 10 carbon atoms, 1 to 6 carbon atoms, or 1 to 4 carbon atoms. Examples of “alkylene” groups include methylene, ethylene, propylene, 1,4-butylene, 1,4-cyclohexylene, and 1,4-cyclohexyldimethylene.

“Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Unless stated otherwise specifically in the specification, an alkyl group can be optionally substituted.

“Alkoxy” refers to the group —OR, where R is alkyl, alkenyl, alkynyl, cycloalkyl, or heterocycle as defined herein. Unless stated otherwise specifically in the specification, alkoxy can be optionally substituted.

The term “backbone” refers to the longest contiguous chain. One or more branches may be covalently bonded to the backbone.

The term “aromatic” refers to a cyclic, fully conjugated planar structure that obeys Hückel's rules, that is the compound has 4n+2π electrons where n is a positive integer or zero. For example, benzene has 6π electrons. Thus, 6=4n+2π. Solving for n gives 1. Therefore, benzene is an aromatic compound.

The term “kosmotrope” is generally used to denote a solute that increases the degree of ordered-ness of water by stabilizing water-water interactions. Kosmotropes may be ionic or non-ionic. In contrast, the term “chaotrope” is generally used to denote a solute that decreases the degree of ordered-ness of water by destabilizing water-water interactions. Chaotropes may be ionic or non-ionic.

The term “peptide” refers to a sequence of amino acid residues without regard to the length of the sequence. Therefore, the term “peptide” refers to any amino acid sequence having at least two amino acids and includes full-length proteins and, as the case may be, polyproteins.

The term “polypeptide” refers to a sequence of amino acid residues without regard to the length of the sequence. Therefore, the term “polypeptide” refers to any amino acid sequence having at least two amino acids and includes full-length proteins, fragments thereof, and/or, as the case may be, polyproteins.

The term “protein” refers to any sequence of two or more amino acid residues without regard to the length of the sequence, as well as any complex of two or more separately translated amino acid sequences. Protein also refers to amino acid sequences chemically modified to include a carbohydrate, a lipid, a nucleotide sequence, or any combination of carbohydrates, lipids, and/or nucleotide sequences. As used herein, “protein,” “peptide,” and “polypeptide” are used interchangeably.

In the description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

### DETAILED DESCRIPTION

The present disclosure provides separation media and separation devices containing the same. Specifically, the disclosure provides separation media that may be used to concentrate or separate (e.g., purify) a target molecule that includes a carbohydrate such as a glycosylated biomolecule. To that end, the separation media of the present disclosure include separation ligands. The separation ligands include a separating group that can be an affinity group, an assistance group, or a capping group. The affinity group includes a carbohydrate binding domain to which the carbohydrate of the target molecule can bind. Multiple layers of separation media of the present disclosure may be arranged in a stacked configuration to increase separation specificity and/or efficiency. The separation media of the present disclosure may be used for separations in membrane chromatography.

A carbohydrate is a monosaccharide, a disaccharide, or a polysaccharide (also called a glycan). Glycans may include a variety of saccharides of varying identities arranged in a glycosylation pattern; that is, the pattern of saccharides in a glycan that includes the identity of the saccharides and the configuration of the connection between adjacent saccharides. Carbohydrate binding proteins can bind to specific glycosylation patterns or a specific saccharide of a glycosylation pattern. Glycans can be conjugated to various bio-molecules to create glyco-biomolecules (i.e., biomolecules that include a glycan group). For example, glycans may be conjugated to lipids, capsids (the protein shell of a virus), and proteins to create glycolipids, glycoproteins, and glycocapsids respectively. A biomolecule that includes a glycan group may be referred to as having been glycosylated (e.g., a glycosylated protein). The binding of various biomolecules to specific saccharides or glycosylation patterns of glycosylated biomolecules regulate many biological processes.

Some viruses include glycosylated surface proteins. The glycosylated surface proteins impact virulence and infectivity of the virus. For example, a viral glycosylated surface protein can bind to host cell surface receptors facilitating entry of the virus into the host cell. Additionally, various proteins (e.g., lectins) can bind to the glycosylated surface protein of a virus to initiate and immune response.

Due to their ability to enter cells, viral vectors derived from viruses are being explored as for delivering gene therapies. A viral vector is a unidirectional non-propagating gene delivery system. The gene of a viral vector is encapsulated in shell. The shell may be a capsid or an envelope (e.g., a lipid bilayer). Viral vectors may include proteins displayed on the surface of the shell such that the proteins are able to interact with proteins and/or other molecules exterior to the viral vector. The proteins displayed on the surface of a viral vector may be bound to shell and/or partially lodged within the shell.

Gene therapy is one of the most promising cure treatments for conditions with very limited or no treatment options, such as rare diseases. The National Institutes of Health reports that nearly 7,000 rare diseases affect more than 25 million Americans, which is about 10% of the US population. In the United States, a rare disease is defined as a condition that affects fewer than 200,000 people. Effective gene therapy is expected to be a single or short-term treatment. To date, there are more than 2,500 gene therapies undergoing clinical trials. More than 90% of the trials are in early stages. Scaling up viral vector production is a critical barrier for completion of clinical trials and successful commercialization for these gene therapies.

Sixty-five percent of the gene therapies under clinical trials are using viral vectors as delivery vehicles. Particularly, the use of lentiviral vectors has been growing rapidly since 2015. While more than 50 different types of vectors are under investigation, 20-30% of the new gene therapy clinical trials are lentiviral vector based. However, due to lentiviral vector fragility and limited purification tools available, downstream purification of these vectors faces significant challenges at larger scale, including poor scalability, low productivity, and often a trade-off between recovery and purity.

Typical viral vector purification methods are time-consuming with many steps and often result in low recovery. For example, to obtain high purity, a typical viral vector downstream process can require as many as nine separate purification steps, including: 1) clarification, 2) benzonase treatment, 3) ion exchange capture, 4) PEG precipitation, 5) centrifugation, 6) solvent extraction, 7) ion exchange polishing, 8) gel filtration, and 9) sterile filtration. This tedious process is costly in terms of time and capital, as well as recovery of active viral vectors. Even if every step could achieve a high recovery of 90%, the final yield after nine steps would be only 39% (0.9⁹×100%=39%). The problem of low recovery is exacerbated by the reality that high starting viral vector titers are difficult to obtain. It is further exacerbated by the fact that viral vectors, such as lentivirus based-viral vectors can lose infectivity during long processing.

Additionally, viral vector purification trains often include various chromatography steps (e.g., ion exchange capture and ion exchange polishing) using one or more types of chromatography columns (e.g., size exclusion columns and affinity chromatography columns). A typical chromatography column used in biomolecule purification (e.g., virus purification) may include a packed bed column with resin configured for size exclusion chromatography, reverse phase chromatography, or affinity chromatography. Resin based chromatography columns have been the gold standard employed to purify biologics for decades. However, resin-based column chromatography in large volumes may be very slow. Resin columns are also known to require long residence times to perform adequately. Furthermore, resin-based systems often have low binding capacity for viral vectors due to the large viron diameter (e.g., ˜100 nm) compared to the pore diameters of the resin. The small pore diameters of resins leads to the exclusion of viral vectors from binding internal resin binding sites. Additionally, traditional elution conditions (e.g., high salt concentrations and/or drastic changes in pH) used for resin-based column chromatography may make the viral vector unstable and result in degradation of the viral vector or recovery of viral vector with decreased infectivity.

Ion-exchange and affinity membrane chromatography columns have been reported to be most effective for lentiviral vector purification. Nevertheless, the current membrane chromatography products all fail to meet the industry needs for high recovery and high purity. Although ion-exchange membrane columns can remove >95% of the impurities, elution of viral vectors requires a high concentration salt buffer or pH change, which have been shown to significantly reduce the infectivity of the viral vector. Lentiviral vectors are especially unstable due to the fragile nature of their envelope. Reported capture step lentiviral vector recovery is usually 50-60%, but falls to 25-30% after an hour storage in the elution buffer. No commercial affinity membrane chromatography columns are available. Heparin-based affinity membranes have been reported for high-selectivity lentiviral vector purification; however, the challenge is that heparin ligands are animal-derived, carrying the risk of contamination. In addition, a recovery of only 53% and 94% impurity removal was reported.

Antibodies and other proteins are rapidly being used and explored as therapeutics. Antibodies are often glycosylated, and the particular glycosylation pattern or presence of terminal glycan residues may impact their ability to bind their partners and/or their half life in the blood plasma. For example, fucose moieties (e.g., in a glycan) on antibodies are through exacerbate immune responses via the opsonin system and result in rapidly cleared drugs. In contrast, the degree of sialyation (i.e., the number of sialic acid groups) of an antibody has been shown to increase the longevity of drug circulation and improve bioavailability, resulting in the ability to apply comparatively lower doses to achieve equitable effect. As such there is need for the ability to rapidly assess the presence and quantity of such glycosylation of antibodies.

Traditionally, antibodies are purified prior to assessing their glycosylation state using high sensitivity instruments such as mass spectrometry. Common methods of assessing the glycosylation state of antibodies make use of high performance liquid chromatograph (HPLC) systems in tandem with mass spec. Theses systems often take hours to run. If species are unable to be resolved with more rapid separation methodologies such as size exclusion chromatography (SEC) due to similar size or property, affinity chromatography may be needed. Currently only resin based or nano porous HPLC columns exist for such purifications. These often necessitate low processing rates or use of high pressures to efficiency perform separations in a timely manner and still require processing times on the order of hours to get samples to adequate purity and perform analytics. Unfortunately, such rates are not amenable to practical, real time sampling of antibody production to assess the glycosylation states of antibodies being produced (e.g., being mass produced in a bioreactor).

The present disclosure describes separation media that may be used for separations in membrane chromatography. In contrast to resin columns, membrane adsorbers perform well at short column residence times, potentially providing rapid separations for biologics. Additionally, membranes may include pore sizes that are larger than the diameter of a viral vector which allows for an increased binding capacity as compared to resin-based chromatography. The separation media of the present disclosure are compatible with non-destructive elution conditions. The present disclosure provides membrane-based separation media that are suitable for non-destructive separation, purification, and/or concentration of biomolecules that include a carbohydrate group. In some embodiments, the separation media of the present disclosure allow for a high recovery of high purity target molecules at low residence times.

Molecules of interest that may be separated using the separation media of the present disclosure are collectively referred to here as target molecules or as targets. The target molecules may be present in a solution, suspension, or dispersion. For simplicity, the liquid containing the target molecule is referred to here as an isolation solution. Also for simplicity, a target may be referred to in the singular but it is understood that an isolation solution may include a plurality of target molecules of the same identity. An isolation solution may also include two or more targets of different identity. The isolation solution may be or include the media or lysate of a recombinant or natural expression system used to make the target molecule. As such, the isolation solution may include other biomolecules or cellular debris. The separation media may be configured for concentrating the target from an isolation solution of already purified target. As such, the isolation solution may include the target molecule, one or more buffering agents, and one or more salts. The isolation solution containing the target molecule may also include solvents, such as water, an organic solvent, or a combination thereof, and soluble components dissolved in the solvent. The separation media may be configured for use with an organic solvent. The separation media may be configured to separate or purify the target molecules from an isolation solution that includes an organic solvent.

A separation media of the present disclosure includes a plurality of separation ligands immobilized on a support substrate. The separation ligands include one or more separation groups and a linker. A separation group is a chemical group that facilitates the isolation of a target molecule from an isolation solution. Facilitation of separation may be in the form of an affinity group to which the target molecule binds or selectively binds; a chemical group to which a target molecule binds to through non-specific interactions (e.g., electrostatic interactions or hydrophobic interactions); a chemical group that allows for increased density of the affinity group—target molecule interaction and/or increases the target molecule attraction to the support substrate; or a chemical group that blocks a reactive group from covalently modifying the target molecule during contact with the separation media; or any combination thereof. A separation group may be an affinity group, an assistance group, or a capping group. The separation media includes a plurality of separation ligands that include an affinity group. In addition to the plurality of separation ligands that include an affinity group, the separation media may include a plurality of separation ligands that include an assistance group; a plurality of separation ligands that include a capping group; or both.

A support substrate is the base material for the separation media. The support substrate provides a platform for which the separation ligands are immobilized. The support substrate includes at least one membrane. In some embodiments, the support substrate is the at least one membrane. In some embodiments, the support substrate includes two or more membranes arranged in a stacked configuration. In addition to the at least one membrane, the support substrate may include additional layers such as hydrogels, woven fibrous materials (i.e., a material made by the interlacing of multiple fibers), nonwoven fibrous materials (i.e., a material made from one or more fibers that are bound together through chemical, physical, heat, or mechanical treatment); or any combination thereof. Such additional layers may impart rigidity and structure to the membrane of the support substrate. In some embodiments, the support substrate includes a functionalized material that is deposited on the surface of the at least one membrane. The functionalized material may provide reactive handles to which the separation ligands may be reacted to be immobilized to the support substrate. In embodiments where the separation media includes multiple layers, the layers may be laminated.

Any layer of the support substrate may be made of any suitable material. A suitable support substrate material is a material that is porous so as to allow the isolation solution to pass through the support substrate. In some embodiments, a suitable support substrate material is a material that does not chemically alter the target molecule; that is, does not react with the target molecule to add, remove, or transform chemical groups on the target molecule. Additionally, in some embodiments, a suitable support substrate is a material that does not react with the target molecule, or other molecules in the isolation solution, to form a covalent bond which would permanently immobilize said molecule to the support substrate.

The support substrate includes at least one membrane. A membrane is understood as a sheet of material with a continuous pathway of polymeric material in all dimensions. The membrane may be made of any suitable support substrate material. Examples of suitable support substrate membrane materials include polyolefins; polyethersulfone; poly(tetrafluoroethylene); nylon; fiberglass; hydrogels; polyvinyl alcohol; natural polymers such as cellulose, cellulose ester, cellulose acetate, regenerated cellulose, cellulosic nanofiber, cellulose derivatives, agarose, chitosan; polyethylene; polyester; polysulfone; expanded polytetrafluoroethylene (ePTFE), polyvinylidene fluoride; polyamide (Nylon); polyacrylonitrile; polycarbonate; and any combination thereof.

In some embodiments, the membrane includes polypropylene. In some embodiments, the polypropylene is microporous polypropylene that has an average pore diameter of 3.0 μm.

In some embodiments, the membrane itself is functionalized prior to immobilizing the separation ligands. Functionalization of the membrane may be done to install reactive handles (e.g., a support substrate reactive handle as discussed herein) on the membrane. The reactive handles react with cooperative reactive handles on the separation ligands to form a covalent bond thereby immobilizing the separation ligands on the support substrate (as discussed herein). Functionalization may be accomplished by plasma treatment, corona treatment, and the like.

In some embodiments, the support substrate includes a functionalized layer. In some embodiments, the functionalized layer is a membrane. A functionalized layer is a material disposed on the surface of a support substrate layer (e.g., disposed on the surface of the at least one membrane) and includes the support substrate reactive handles that may be used for separation ligand immobilization. A functionalized layer may be covalently attached to the support substrate; adhered to the support substrate through electrostatic forces, hydrogen-bonding, and/or Van der Waals forces; laminated to the support substrate; or simply contacting the support substrate. A functionalized layer may be deposited on the surface a support substrate (e.g., on the surface of the at least one membrane) using a variety of deposition techniques such as chemical vapor deposition, dip coating, spray coating, electrospinning, and the like.

In some embodiments, the functionalized layer is a polymer that is disposed onto the support surface using a grafting on or grafting from polymerization technique. The terms “grafting on,” “grafting onto,” and “grafted onto” refer to already formed polymer chains that adsorb or covalently attach to a surface (e.g., a support substrate surface). The terms “grafting from” or “grafted from” refer to a polymer chain that is imitated and grown from a surface (e.g., a support substrate surface). Any suitable polymer may be grafted on or grafted from a support substrate to from a functionalized layer. Suitable polymers are those that include a functional group that includes a reactive handle that allows for attachment of separation ligands to the support substrate. The reactive handle is not the polymerizable group, but instead is a group that remains intact following polymerization. Example polymers that include a reactive handle or a functional group that can be converted to a reactive handle (i.e., support substrate reactive handle) include carboxylic acids, amines, alcohols, epoxides, amides, azide, alkynes, and the like. Examples of monomers that can be used to form such polymers include vinyl alcohol, hydroxy functional acrylates (e.g., 2-hydroxyethyl acrylate and 4-hydroxybutyl acrylate), hydroxy functional methacrylate (e.g., hydroxyethyl methacrylate), epoxy containing monomers, and hydroxy functional acrylamides (e.g., N-hydroxyethyl acrylamide). Examples of specific polymers that may be grafted on or grafted from a support substrate include polydopamine, poly(vinyl alcohol), poly(acrylic acid), poly(glycidyl methacrylate, and poly 2-hydroxyethyl acrylate (formed from 2-hydroxyethyl acrylate monomers). Graft on and graft from polymerization may be accomplished using suitable technique such addition polymerization (e.g., free radical polymerization such as atom transfer radical polymerization (ATRP) and reversible addition fragmentation chain transfer (RAFT) polymerization; anionic polymerization; and cationic polymerization) or condensation polymerization. In embodiments, where the polymer is grafted from the support substrate, an initiator is first coupled to the support substrate (e.g., through an OH group on the support substrate). Any suitable initiator may be used, for example, 2-bromo-2-methylpropionyl bromide (BiBB).

The membranes of the support substrate are porous and can have an average pore size, as measure by a capillary flow porometer, of 10 micrometer (μm) or less, 5 μm or less, 2 μm or less, 1 μm or less, 0.6 μm or less, 0.5 μm or less, 0.45 μm or less, or 0.2 μm or less. The membrane may have an average pore size of 0.1 μm or greater, 0.2 μm or greater, 0.45 μm or greater, 0.5 μm or greater, 0.6 μm or greater, 0.7 μm or greater, or 1 μm or greater. The membrane may have an average pore size ranging from about 0.1 μm to 10.0 μm, 0.1 μm to 0.2 μm, 0.1 μm to 0.45 μm, 0.1 μm to 0.5 μm, 0.1 μm to 1 μm, 0.2 μm to 0.45, 0.2 μm to 0.50, 0.2 μm to 1 μm, 0.2 μm to 2 μm, 0.2 μm to 10 μm, 0.45 μm to 1 μm, 0.45 μm to 2 μm, 0.45 μm to 10 μm, 1 μm to 2 μm, or 1 μm to 5 μm. In some embodiments, the support substrate has an average pore size of 0.1 μm to 0.5 μm, 0.1 μm to 0.6 μm, 0.1 μm to 0.3 μm, or 0.4 μm to 0.6 μm.

The membrane of the support substrate (each membrane) may have a thickness of 500 μm or greater, 250 μm or greater, 100 μm or greater, 80 μm or greater, 50 μm or greater, or 30 μm or greater. The membrane may have a thickness of 2500 μm or less, 1000 μm or less, 500 μm or less, 250 μm or less, or 100 μm or less. The thickness of the membrane may be in a range of 30 μm to 500 μm, 50 μm to 500 μm, 80 μm to 500 μm, 100 μm to 500 μm, 250 μm to 500 μm, 30 μm to 250 μm, 50 μm to 250 μm, 80 μm to 250 μm, 100 μm to 2500 μm, 30 μm to 100 μm, 50 μm to 100 μm, or 80 μm to 100 μm.

In some embodiments, the support substrate includes multiple membranes stacked in a multilayer arrangement to increase capacity or selectivity of the separation media for a given application. The multilayer membrane configuration (i.e., only considering the membrane layers of a support substrate) maybe have a thickens of 10,000 μm or less, 7,500 μm or less, 5,000 μm or less, 4,000 μm or less, 3,000 μm or less, 2,500 μm or less, 2,000 μm or less, 1,000 μm or less, 750 μm or less, 500 μm or less, 400 μm or less, or 300 μm or less. The stacked arrangement of membranes may have a thickness ranging from 70 μm to 10,000 μm, 70 μm to 100 μm, 70 μm to 200 μm, 70 μm to 300 μm, 70 μm to 400 μm, 70 μm to 500 μm, 70 μm to 750 μm, 70 μm to 1,000 μm, 70 μm to 2,000 μm, 70 μm to 3,000 μm, 70 μm to 4,000 μm, 70 μm to 5,000 μm, 250 μm to 300 μm, 250 μm to 400 μm, 250 μm to 500 μm, 250 μm to 750 μm, 250 μm to 1,000 μm, 250 to 2,000 μm, 250 to 3,000 μm, 250 to 4,000 μm, 250 to 5,000 μm, 500 μm to 1,000 μm, 500 μm to 2,000 μm, 500 μm to 3,000 μm, 500 μm to 4,000 μm, or 500 μm to 5,000 μm in thickness.

In some embodiments, the membrane is a regenerated cellulose membrane having a pore size of between 0.2 μm and 5.0 μm, a thickness of between 70 μm and 2,000 μm. Such membranes may be in a stacked arrangement approximately 70 μm to 10,000 μm in thickness.

In some embodiments, the support membrane includes cellulose such as regenerated cellulose, cellulose acetate, or cellulose ester. In some such embodiments, the support membrane has an average pore size 0.1 μm to 0.5 μm, 0.1 μm to 0.6 μm, 0.1 μm to 0.3 μm, or 0.4 μm to 0.6 μm.

The support substrate may include or be a microfiltration membrane. Microfiltration membranes are typically created through a phase inversion process or an expansion process. Typical materials used to prepare membranes include polyethersulfone (PES), nylon, polyvinylidene fluoride (PVDF), cellulose acetate, regenerated cellulose, polypropylene, and expanded polytetrafluoroethylene (ePTFE).

The separation media includes a plurality of separation ligands that include an affinity group. In addition to the plurality of separation ligands that include an affinity group, the separation media may include a plurality of separation ligands that include an assistance group; a plurality of ligands that include a capping group; or both.

An affinity group is a chemical group that is bound by the target molecule. The affinity group may include a carbohydrate binding ligand or a carbohydrate binding domain. The term “carbohydrate binding ligand” refers to a small molecule that is capable of recognizing and binding to a carbohydrate. The term “carbohydrate binding domain” refers to a biomolecule, such as a protein, a peptide, or a portion thereof, that is capable of recognizing and binding to a carbohydrate. The target may be a viral vector or a free protein that includes a carbohydrate to which the carbohydrate binding ligand and/or carbohydrate binding domain can bind thereby temporarily immobilizing the target molecule to the separation media.

Carbohydrate is the general term for the complete complement of sugars in an organism and includes monosaccharides (a single saccharide), disaccharides (two covalently linked saccharides), and glycans (a polymer of three or more saccharides). A monosaccharide is the simplest carbohydrate form. Monosaccharides can have various chemical compositions and configurations. Generally, a monosaccharide includes carbon, hydrogen, and oxygen atoms often according to the empirical formula Cₘ(H₂O)ₙ where m and n are integers that may or may not be the same (i.e., the hydrogen-oxygen ratio is 2:1). However, not all monosaccharides follow the empirical formula such as, for example, uronic acid and deoxy-sugars (e.g., fucose, deoxyribose, fuculose, and rhamnose), and dideoxy sugars (e.g., colitose and abequose). Additionally, monosaccharides may include one or more substituents or modifications that make it such that the monosaccharide no longer follows the empirical formula. Example substituent groups and modifications include acetyl (Ac); D-alanyl (Ala), N-acetyl-D-alanyl (Ala2Ac); N-acetimidoyl (Am); N—(N-methyl-acetimidoyl) (AmMe); N—(N,N-dimethyl-acetimidoyl) (AmMe2); formyl (Fo); glycolyl (Gc); N-acetyl-glutaminyl (Gln2Ac); N-methyl-5-glutamyl (5Glu2Me); glycyl (Gly); glyceryl (Gr); 2,3-di-O-methyl-glyceryl (Gr2,3Me₂); 4-hydroxybutyryl (4Hb); 3,4-dihydroxybutyryl (3,4Hb); (R)-3-hydroxybutyryl (3_{R}Hb); (S)-3-hydroxybutyryl (3_{S}Hb); lactyl (Lt); methyl (Me); amino (N); N-acetyl (NAc); phosphate (P); pyruvyl (Py); 1-carboxyethylidene (Pyr); sulfate (S); and tauryl (Tau). Table 1 shows examples of monosaccharides.

Each monosaccharide may have a variety of stereoisomers. For example, monosaccharides are often classified as D or L depending on the stereochemistry of the stereocenter that is the farthest away from the anomeric carbon. Additionally, monosaccharides can exist as anomers (α-anomer and β-anomer). An anomer is a pair of stereoisomers that are identical accept at the configuration of the anomeric carbon. A single monosaccharide may exist as an α-anomer or a β-anomer.

TABLE 1
Example monosaccharides

| Monosaccharide name | Monosaccharide name | Monosaccharide name |
|---|---|---|
| 4-epi-Legionaminic acid | L-Fucose | N-Acetyl-D-mannosamine |
| 6-Deoxy-L-altrose | N-Acetyl-L-fucosamine | Muramic acid |
| N-Acetyl-6-deoxy-L- | D-Galactose | N-Acetylmuramic acid |
| altrosamine | | |
| 6-Deoxy-D-gulose | D-Galacturonic acid | N-Glycolylmuramic acid |
| 6-Deoxy-D-talose | D-Galactosamine | Neuraminic acid |
| N-Acetyl-6-deoxy-D- | N-Acetyl-D-galactosamine | N-Acetylneuraminic acid |
| talosamine | | |
| 8-epi-Acinetaminic acid | D-Glucose | N-Glycolylneuraminic acid |
| 8-epi-Legionaminic acid | D-Glucuronic acid | Olivose |
| Abequose | D-Glucosamine | Paratose |
| Acinetaminic acid | N-Acetyl-D-glucosamine | Pseudaminic acid |
| D-Allose | D-Gulose | D-Psicose |
| D-Alluronic acid | D-Guluronic acid | D-Quinovose |
| D-Allosamine | D-Gulosamine | N-Acetyl-D-quinovosamine |
| N-Acetyl-D-allosamine | N-Acetyl-D-gulosamine | L-Rhamnose |
| L-Altrose | L-Idose | N-Acetyl-L-rhamnosamine |
| L-Altruronic acid | L-Iduronic acid | D-Ribose |
| L-Altrosamine | L-Idosamine | Sialic acid |
| N-Acetyl-L-altrosamine | N-Acetyl-L-idosamine | L-Sorbose |
| L-Apiose | 2-Keto-3-deoxy-nononic acid | D-Tagatose |
| L-Arabinose | 3-Deoxy-D-manno-octulosonic acid | D-Talose |
| Bacillosamine | Legionaminic acid | D-Taluronic acid |
| Colitose | L-glycero-D-manno-Heptose | D-Talosamine |
| D-glycero-D-manno-Heptose | D-Lyxose | N-Acetyl-D-talosamine |
| 3-Deoxy-D-lyxo- | D-Mannose | Tyvelose |
| heptulosaric acid | | |
| D-Digitoxose | D-Mannuronic acid | D-Xylose |
| D-Fructose | D-Mannosamine | |

Monosaccharides may be covalently linked to form larger molecules such as disaccharides or glycans. Monosaccharides are linked through glycosidic bonds; that is, covalent bonds between adjacent monosaccharides of a disaccharide or glycan. Glycosidic bonds may also be used to describe a monosaccharide or a glycan that is covalently linked to a different molecule such as a protein. In disaccharides and glycans, a glycosidic linkage couples the anomeric carbon of a first saccharide to a carbon of an adjacent saccharide through and O-type (ether linkage), N-type (amine linkage), or S-type (thioether linkage) linkage. Glycosidic linkages may take on several configurations depending on how two monosaccharides are connected. The configuration of the linkage is denoted by listing the anomeric carbon number from which the bond originates first followed by the carbon number of the second monosaccharide to which the anomeric carbon of the first monosaccharide is linked to by a glycosidic linkage. For example, a first monosaccharide and a second monosaccharide may have a C1 to C4 glycosidic linkage; that is, the anomeric carbon (C1) of the first monosaccharide is coupled to the carbon in position 4 (C4) of the second monosaccharide. Additionally, the stereochemistry configuration of the glycosidic linkage may vary depending on the configuration of the anomeric carbon that covalently links the first monosaccharide to the second monosaccharide through the glycosidic linkage. If the first monosaccharide is an α-anomer, the glycosidic linkage is and a linkage. If the first monosaccharide is a β-anomer, the glycosidic linkage is a 3 linkage.

Sialic acids are a class of saccharides (a subclass of nonulsonic acids) that help regulate cell-cell interaction, cell signaling, carbohydrate-cell interactions, cell aggregation, immune reactions, reproduction, and developmental processes. Additionally, sialic acids have been found to enable infectious disease infection (e.g., viruses and bacteria) and tumor growth. Sialic acids (also called neuraminic acids) are alpha-keto acid (possess a ketone and carboxylic acid group) saccharides that have a nine carbon backbone. There are numerous sialic acids that differ in structure. Examples of sialic acid monosaccharides are shown in Table 2. Additionally, there are “sialic acid-like” monosaccharides which are dideoxy-nonulosonic acids (deoxy at C3 and C 9). Examples of sialic acid like monosaccharides are given in Table 3.

TABLE 2
Example sialic acid monosaccharides

| Sialic Acid Name | Abbreviation |
|---|---|
| 5-Amino-3,5-dideoxy-D-glycero-D-galacto-non-2-ulosonic acid/ | Neu |
| neuraminic acid | |
| Neuraminic acid 1,5-lactam | Neu1, 5lactam |
| 5-N-Acetyl-neuraminic acid (N-Acetylneuraminic acid) | Neu5Ac |
| 5-N-Acetyl-4-O-acetyl-neuraminic acid | Neu4, 5Ac2 |
| 5-N-Acetyl-7-O-acetyl-neuraminic acid | Neu5, 7Ac2 |
| 5-N-Acetyl-8-O-acetyl-neuraminic acid | Neu5, 8Ac2 |
| 5-N-Acetyl-9-O-acetyl-neuraminic acid | Neu5, 9Ac2 |
| 5-N-Acetyl-4,9-di-O-acetyl-neuraminic acid | Neu4, 5, 9Ac3 |
| 5-N-Acetyl-7,8-di-O-acetyl-neuraminic acid | Neu5, 7, 8Ac3 |
| 5-N-Acetyl-7,9-di-O-acetyl-neuraminic acid | Neu5, 7, 9Ac3 |
| 5-N-Acetyl-8,9-di-O-acetyl-neuraminic acid | Neu5, 8, 9Ac3 |
| 5-N-Acetyl-4,7,9-tri-O-acetyl-neuraminic acid | Neu4, 5, 7, 9Ac4 |
| 5-N-Acetyl-7,8,9-tri-O-acetyl-neuraminic acid | Neu5, 7, 8, 9Ac4 |
| 5-N-Acetyl-4,7,8,9-tetra-O-acetyl-neuraminic acid | Neu4, 5, 7, 8, 9Ac5 |
| 5-N-Acetyl-4-O-glycolyl-neuraminic acid | Neu5Ac4Gc |
| 5-N-Acetyl-7-O-glycolyl-neuraminic acid | Neu5Ac7Gc |
| 5-N-Acetyl-9-O-lactyl-neuraminic acid | Neu5Ac9Lt |
| 5-N-Acetyl-4-O-acetyl-9-O-lactyl-neuraminic acid | Neu4, 5Ac29Lt |
| 5-N-Acetyl-7-O-acetyl-9-O-lactyl-neuraminic acid | Neu5, 7Ac29Lt |
| 5-N-Acetyl-8-O-acetyl-9-O-lactyl-neuraminic acid | Neu5, 8Ac29Lt |
| 5-N-Acetyl-8-O-methyl-neuraminic acid | Neu5Ac8Me |
| 5-N-Acetyl-4-O-acetyl-8-O-methyl-neuraminic acid | Neu4, 5Ac28Me |
| 5-N-Acetyl-9-O-acetyl-8-O-methyl-neuraminic acid | Neu5, 9Ac28Me |
| 5-N-Acetyl-9-O-methyl-neuraminic acid | Neu5Ac9Me |
| 5-N-Acetyl-4-O-sulpho-neuraminic acid | Neu5Ac4S |
| 5-N-Acetyl-8-O-sulpho-neuraminic acid | Neu5Ac8S |
| 5-N-Acetyl-4-O-acetyl-8-O-sulpho-neuraminic acid | Neu4, 5Ac28S |
| 5-N-Acetyl-9-O-phospho-neuraminic acid | Neu5Ac9P |
| 5-N-Acetyl-2-deoxy-2,3-didehydro-neuraminic acid | Neu2en5Ac |
| 5-N-Acetyl-9-O-acetyl-2-deoxy-2,3-didehydro-neuraminic acid | Neu2en5, 9Ac2 |
| 5-N-Acetyl-2-deoxy-2,3-didehydro-9-O-lactyl-neuraminic acid | Neu2en5Ac9Lt |
| 5-N-Acetyl-2,7-anhydro-neuraminic acid | Neu2, 7an5Ac |
| 5-N-Acetyl-4,8-anhydro-neuraminic acid | Neu4, 8an5Ac |
| 5-N-Acetyl-neuraminic acid 1,7-lactone | Neu1, 7lactone5Ac |
| 5-N-Acetyl-9-O-acetyl-neuraminic acid 1,7-lactone | Neu1, 7lactone5, 9Ac2 |
| 5-N-Acetyl-4,9-di-O-acetyl-neuraminic acid 1,7-lactone | Neu1, 7lactone4, 5, 9Ac3 |
| 1-Tauryl 5-N-acetyl-neuraminic amide | Neu5Ac1Tau |
| 5-N-Glycolyl-neuraminic acid (N-Glycolylneuraminic acid) | Neu5Gc |
| 4-O-Acetyl-5-N-glycolyl-neuraminic acid | Neu4Ac5Gc |
| 7-O-Acetyl-5-N-glycolyl-neuraminic acid | Neu7Ac5Gc |
| 8-O-Acetyl-5-N-glycolyl-neuraminic acid | Neu8Ac5Gc |
| 9-O-Acetyl-5-N-glycolyl-neuraminic acid | Neu9Ac5Gc |
| 4,7-Di-O-acetyl-5-N-glycolyl-neuraminic acid | Neu4, 7Ac25Gc |
| 4,9-Di-O-acetyl-5-N-glycolyl-neuraminic acid | Neu4, 9Ac25Gc |
| 7,9-Di-O-acetyl-5-N-glycolyl-neuraminic acid | Neu7, 9Ac25Gc |
| 8,9-Di-O-acetyl-5-N-glycolyl-neuraminic acid | Neu8, 9Ac25Gc |
| 4,7,9-Tri-O-acetyl-5-N-glycolyl-neuraminic acid | Neu4, 7, 9Ac35Gc |
| 7,8,9-Tri-O-acetyl-5-N-glycolyl-neuraminic acid | Neu7, 8, 9Ac35Gc |
| 4,7,8,9-Tetra-O-acetyl-5-N-glycolyl-neuraminic acid | Neu4, 7, 8, 9Ac45Gc |
| 5-N-Glycoly1-9-O-lactyl-neuraminic acid | Neu5Gc9Lt |
| 4-O-Acetyl-5-N-glycolyl-9-O-lactyl-neuraminic acid | Neu4Ac5Gc9Lt |
| 7-O-Acetyl-5-N-glycolyl-9-O-lactyl-neuraminic acid | Neu7Ac5Gc9Lt |
| 8-O-Acetyl-5-N-glycolyl-9-O-lactyl-neuraminic acid | Neu8Ac5Gc9Lt |
| 4,7-Di-O-acetyl-5-N-glycoly1-9-O-lactyl-neuraminic acid | Neu4, 7Ac25Gc9Lt |
| 7,8-Di-O-acetyl-5-N-glycolyl-9-O-lactyl-neuraminic acid | Neu7, 8Ac25Gc9Lt |
| 5-N-Glycolyl-8-O-methyl-neuraminic acid | Neu5Gc8Me |
| 4-O-Acetyl-5-N-glycolyl-8-O-methyl-neuraminic acid | Neu4Ac5Gc8Me |
| 7-O-Acetyl-5-N-glycolyl-8-O-methyl-neuraminic acid | Neu7Ac5Gc8Me |
| 9-O-Acetyl-5-N-glycolyl-8-O-methyl-neuraminic acid | Neu9Ac5Gc8Me |
| 4,7-Di-O-acetyl-5-N-glycoly1-8-O-methyl-neuraminic acid | Neu4, 7Ac25Gc8Me |
| 7,9-Di-O-acetyl-5-N-glycolyl-8-O-methyl-neuraminic acid | Neu7, 9Ac25Gc8Me |
| 5-N-Glycolyl-9-O-methyl-neuraminic acid | Neu5Gc9Me |
| 5-N-Glycoly1-8-O-sulpho-neuraminic acid | Neu5Gc8S |
| 5-N-Glycoly1-9-O-sulpho-neuraminic acid | Neu5Gc9S |
| 5-N-(O-Acetyl)glycolyl-neuraminic acid | Neu5(Gc2Ac) |
| 5-N-(O-Methyl)glycolyl-neuraminic acid | Neu5(Gc2Me) |
| 2-Deoxy-2,3-didehydro-5-N-glycolyl-neuraminic acid | Neu2en5Gc |
| 9-O-Acetyl-2-deoxy-2,3-didehydro-5-N-glycolyl-neuraminic acid | Neu2en9Ac5Gc |
| 2-Deoxy-2,3-didehydro-5-N-glycolyl-9-O-lactyl-neuraminic acid | Neu2en5Gc9Lt |
| 2-Deoxy-2,3-didehydro-5-N-glycolyl-8-O-methyl-neuraminic acid | Neu2en5Gc8Me |
| 2,7-Anhydro-5-N-glycolyl-neuraminic acid | Neu2, 7an5Gc |
| 2,7-Anhydro-5-N-glycolyl-8-O-methyl-neuraminic acid | Neu2, 7an5Gc8Me |
| 4,8-Anhydro-5-N-glycolyl-neuraminic acid | Neu4, 8an5Gc |
| 5-N-Glycolyl-neuraminic acid 1,7-lactone | Neu1, 7lactone5Gc |
| 9-O-Acetyl-5-N-glycolyl-neuraminic acid 1,7-lactone | Neu1, 7lactone9Ac5Gc |
| 7-Acetamido-9-O-acetyl-7-deoxy-5-N-glycolyl-neuraminic acid | Neu9Ac5Gc7NAc |
| 7-Acetamido-8,9-di-O-acetyl-7-deoxy-5-N-glycolyl-neuraminic acid | Neu8, 9Ac25Gc7NAc |
| 3-Deoxy-D-glycero-D-galacto-non-2-ulosonic acid/2-keto-3- | Kdn |
| deoxy-nononic acid | |
| 5-O-Acetyl-2-keto-3-deoxy-nononic acid | Kdn5Ac |
| 7-O-Acetyl-2-keto-3-deoxy-nononic acid | Kdn7Ac |
| 8-O-Acetyl-2-keto-3-deoxy-nononic acid | Kdn8Ac |
| 9-O-Acetyl-2-keto-3-deoxy-nononic acid | Kdn9Ac |
| 4,5-Di-O-acetyl-2-keto-3-deoxy-nononic acid | Kdn4, 5Ac2 |
| 4,7-Di-O-acetyl-2-keto-3-deoxy-nononic acid | Kdn4, 7Ac2 |
| 5,9-Di-O-acetyl-2-keto-3-deoxy-nononic acid | Kdn5, 9Ac2 |
| 7,9-Di-O-acetyl-2-keto-3-deoxy-nononic acid | Kdn7, 9Ac2 |
| 8,9-Di-O-acetyl-2-keto-3-deoxy-nononic acid | Kdn8, 9Ac2 |
| 2-Keto-3-deoxy-4-O-methyl-nononic acid | Kdn4Me |
| 2-Keto-3-deoxy-5-O-methyl-nononic acid | Kdn5Me |
| 2-Keto-3-deoxy-9-O-methyl-nononic acid | Kdn9Me |
| (R)-7,9-O-[1-Carboxyethylidene]-2-keto-3-deoxy-nononic acid | Kdn7, 9PyrR |
| 2-Keto-3-deoxy-9-O-phospho-nononic acid | Kdn9 |

TABLE 3
Example sialic acid like monosaccharides

| Sialic Acid-Like Monosaccharide Name | Abbreviation |
|---|---|
| 5,7-Diamino-3,5,7,9-tetradeoxy-L-glycero-L-manno-non- | Pse |
| 2-ulosonic acid/pseudaminic acid | |
| 5,7-Di-N-acetyl-pseudaminic acid | Pse5, 7Ac2 |
| 5,7-Di-N-acetyl-4-O-acetyl-pseudaminic acid | Pse4, 5, 7Ac3 |
| 5,7-Di-N-acetyl-8-O-acetyl-pseudaminic acid | Pse5, 7, 8Ac3 |
| 5,7-Di-N-acetyl-8-O-glycyl-pseudaminic acid | Pse5, 7Ac28Gly |
| 5,7-Di-N-glyceryl-pseudaminic acid | Pse5, 7Gr2 |
| 5-N-Acetimidoyl-7-N-acetyl-pseudaminic acid | Pse7Ac5Am |
| 5-N-Acetimidoy1-7-N-acetyl-8-O-acetyl-pseudaminic acid | Pse7, 8Ac25Am |
| 5-N-Acetimidoy1-7-N-acetyl-8-O-(N-acetyl-glutaminyl)- | Pse7Ac5Am8(Gln2Ac) |
| pseudaminic acid | |
| 5-N-Acetyl-7-N-formyl-pseudaminic acid | Pse5Ac7Fo |
| 5-N-Acetyl-7-N-L-glyceryl-pseudaminic acid | Pse5Ac7Gr |
| 5-N-Acetyl-7-N-[(R)-3-hydroxybutyryl]-pseudaminic acid | Pse5Ac7(3RHb) |
| 5-N-Acetyl-7-N-[(R)-3-hydroxybutyry1]-4-O-acetyl- | Pse4, 5Ac27(3RHb) |
| pseudaminic acid | |
| 5-N-Acetyl-7-N-[(S)-3-hydroxybutyryl]-pseudaminic acid | Pse5Ac7(3SHb) |
| 5-N-Acetyl-7-N-(4-hydroxybutyryl)-pseudaminic acid | Pse5Ac7(4Hb) |
| 5-N-Acetyl-7-N-(3,4-dihydroxybutyryl)-pseudaminic acid | Pse5Ac7(3, 4Hb) |
| 7-N-Acetimidoyl-5-N-acetyl-pseudaminic acid | Pse5Ac7Am |
| 5-N-Acetimidoyl-7-N-glyceryl-pseudaminic acida | Pse5Am7Gr |
| 7-N-Acetimidoyl-5-N-(2,3-di-O-methyl-glyceryl)- | Pse7Am5(Gr2, 3Me2) |
| pseudaminic acid | |
| 7-N-Acetyl-5-N-(3-hydroxybutyryl)-pseudaminic acid | Pse7Ac5(3Hb) |
| 7-N-Acetyl-5-N-(2,3-di-O-methyl-glyceryl)-pseudaminic | Pse7Ac5(Gr2, 3Me2) |
| acid | |
| 7-N-Formy1-5-N-[(R)-3-hydroxybutyryl]-pseudaminic acid | Pse7Fo5(3RHb) |
| 5,7-Diamino-3,5,7,9-tetradeoxy-D-glycero-D-galacto-non- | Leg |
| 2-ulosonic acid/legionaminic acid | |
| 5,7-Di-N-acetyl-legionaminic acid | Leg5, 7Ac2 |
| 5,7-Di-N-acetyl-4-O-acetyl-legionaminic acid | Leg4, 5, 7Ac3 |
| 5,7-Di-N-acetyl-8-amino-8-deoxy-legionaminic acid | Leg5, 7Ac28N |
| 5-N-Acetimidoyl-7-N-acetyl-legionaminic acid | Leg7Ac5Am |
| 5-N-Acetimidoy1-7-N-acetyl-8-O-acetyl-legionaminic acid | Leg7, 8Ac25Am |
| 5-N-Acetimidoy1-7-N-acetyl-5-N-methyl-legionaminic | Leg7Ac5Am5Me |
| acid | |
| 5-N-(N-Methyl-acetimidoyl)-7-N-acetyl-legionaminic acid | Leg7Ac5AmMe |
| 5-N-(N,N-Dimethyl-acetimidoy1)-7-N-acetyl-legionaminic | Leg7Ac5AmMe2 |
| acid | |
| 5-N-Acetimidoyl-7-N-acetyl-8-O-acetyl-5-N-methyl- | Leg7, 8Ac25Am5Me |
| legionaminic acid | |
| 5-N-(N,N-Dimethyl-acetimidoyl)-7-N-acetyl-8-O-acetyl- | Leg7, 8Ac25AmMe2 |
| legionaminic acid | |
| 5-N-Acetyl-7-N-(N-acetyl-D-alanyl)-legionaminic acid | Leg5Ac7(Ala2Ac) |
| 5-N-Acetyl-7-N-(D-alanyl)-legionaminic acid | Leg5Ac7Ala |
| 7-N-Acetyl-5-N-formyl-legionaminic acid | Leg7Ac5Fo |
| 7-N-Acetyl-5-N-[(S)-3-hydroxybutyryl]-legionaminic | Leg7Ac5(3SHb) |
| acidd | |
| 7-N-Acetyl-5-N-(N-methyl-5-glutamyl)-legionaminic acid | Leg7Ac5(5Glu2Me) |
| 5,7-Diamino-3,5,7,9-tetradeoxy-D-glycero-D-talo-non-2- | 4Leg |
| ulosonic acid/4-epi-legionaminic acid | |
| 5,7-Di-N-acetyl-4-epi-legionaminic acid | 4eLeg5, 7Ac2 |
| 5,7-Di-N-acetyl-8-O-acetyl-4-epi-legionaminic acid | 4eLeg5, 7, 8Ac3 |
| 5-N-Acetimidoyl-7-N-acetyl-4-epi-legionaminic acid | 4eLeg7Ac5Am |
| 5-N-Acetimidoyl-7-N-acetyl-8-O-acetyl-4-epi- | 4eLeg7, 8Ac25Am |
| legionaminic acid | |
| 5,7-Diamino-3,5,7,9-tetradeoxy-L-glycero-D-galacto-non- | 8eLeg |
| 2-ulosonic acid/8-epi-legionaminic acid | |
| 5,7-Di-N-acetyl-8-epi-legionaminic acid | 8eLeg5, 7Ac2 |
| 5,7-Di-N-acetyl-8-O-acetyl-8-epi-legionaminic acid | 8eLeg5, 7, 8Ac3 |
| 5-N-Acetimidoy1-7-N-acetyl-8-epi-legionaminic acid | 8eLeg7Ac5Am |
| 7-N-Acetimidoy1-5-N-acetyl-8-epi-legionaminic acid | 8eLeg5Ac7Am |
| 7-N-Acetimidoy1-5-N-acetyl-8-O-acetyl-8-epi- | 8eLeg5, 8Ac27Am |
| legionaminic acid | |
| 7-N-Acetyl-5-N-[(R)-3-hydroxybutyryl]-8-epi- | 8eLeg7Ac5(3RHb) |
| legionaminic acid | |
| 7-N-Acetyl-5-N-(4-hydroxybutyryl)-8-epi-legionaminic | 8eLeg7Ac5(4Hb) |
| acid | |
| 5,7-Diamino-3,5,7,9-tetradeoxy-L-glycero-L-altro-non-2- | Aci |
| ulosonic acid/acinetaminic acid | |
| 5,7-Di-N-acetyl-acinetaminic acid | Aci5, 7Ac2 |
| 5,7-Diamino-3,5,7,9-tetradeoxy-D-glycero-L-altro-non-2- | 8eAci |
| ulosonic acid/8-epi-acinetaminic acid | |
| 5,7-Di-N-acetyl-8-epi-acinetaminic acid | 8eAci5, 7Ac2 |
| Some related 9-deoxy-non-2-ulosonic acids | |
| 5- or 7-Acetamido-, 7- or 5-(3-hydroxybutyramido)-5,7,9- | |
| trideoxy-non-2-ulosonic acid | |
| 5-Acetamido-7-[(S)-3-hydroxybutyramido]-8-amino- | |
| 3,5,7,8,9-pentadeoxy-L-glycero-L-manno- or D-glycero-L- | |
| manno-non-2-ulosonic acid | |
| 5-Acetamidino-3,5,9-trideoxy-L-glycero-L-gluco-non-2- | |
| ulosonic acid (tentatively assigned chirality; trivial name: | |
| fusaminic acid) | |
| 5-Acetamidino-4-O-acetyl-3,5,9-trideoxy-L-glycero-L- | |
| gluco-non-2-ulosonic acid (tentatively assigned chirality) | |
| 5-Acetamidino-7-acetamido-3,5,7,9-tetradeoxy-D-glycero- | |
| L-gluco-non-2-ulosonic acid (tentatively assigned chirality) | |

Sialic acids may be terminal or internal residues of a glycan. Most commonly, sialic acids are terminal residues of cell surface glycans. As such, sialic acids help to regulate various biological processes including cell-cell signaling, tumor growth and metathesis, cell adhesion, and cellular recognition. Additionally, viruses, such as adeno-assisted viruses can bind to cell surface sialyated glycans (a glycan that includes at least one sialic acid saccharide) and achieve entry into a host cell. Furthermore, lectins may bind to sialyated glycans on biomolecules to initiate downstream biological processes.

The monosaccharide composition and the configuration of the linkage of the monosaccharaides leads to polysaccharides with higher order structure. Disaccharides and polysaccharides may be homopolysaccharides (or homoglycans) or may be heterosaccharides (or heteroglycans). Homopolysaccharides include a single type of monosaccharide. In contrast, heteropolysaccharides include two or more different types of monosaccharides. Glycans may be in a linear or branched configuration. A linear glycan has a straight chain of linked monosaccharides. Examples of linear glycans include cellulose and chitin. A branched glycan is a glycan that has a glycan backbone and one or more branches off the glycan backbone.

In some embodiments, the target is a virus or a viral vector. In some embodiments, the viral vector includes one or more surface proteins that include a carbohydrate. In some embodiments, the one or more surface proteins of the viral vector are glycosylated (a glycosylated surface protein). In some embodiments, the target molecule is a virus or a viral vector that includes a glycosylated surface protein. In some embodiments, the virus or viral vector is a lentiviral vector, an adenoviral vector, or an adeno-associated viral vector. In some embodiments, the virus is a lentiviral vector, and the lentiviral vector is a human immunodeficiency-virus (HIV) based viral vector.

The glycosylated surface protein of the target viral vector may be any natural or engineered glycosylated surface protein. Examples of glycosylated surface proteins of viruses that may be used in viral vectors include, stomatitis virus glycoprotein (VSV-G), envelope glycoprotein GP120, envelope glycoprotein GP160, envelope glycoprotein G41, hemagglutinin, neuraminidase, spike(S) glycoprotein, hepatitis C virus envelope glycoprotein E1, hepatitis C virus envelope glycoprotein E2, Zaire Ebola virus spike protein Gp1, Zaire Ebola virus spike protein Gp2, engineered variants thereof, or any combination thereof.

The glycosylated surface protein of the target viral vector includes at least one conjugated glycan. The glycan may include any glycosylation pattern; that is, any pattern of monosaccharides, each monosaccharide linked to at least one other monosaccharide through a glycosidic linkage. The monosaccharides included in a glycan may be any monosaccharide as described herein including stereoisomers thereof, and ionized versions thereof, as well as have any substituent or modification as described herein. Additionally, each glycosidic linkage in a glycan affinity group may be of any configuration as described herein.

In some embodiments, the affinity group includes a carbohydrate binding domain capable of binding to a glycosylated surface protein of a target molecule. Specifically, the carbohydrate binding domain binds to a specific monosaccharide of the glycan of the glycosylated viral vector surface protein; binds to a specific glycosylation pattern of the glycan of the glycosylated viral vector surface protein; or both. The carbohydrate domain can be chosen based on the target molecule to be isolated. In some embodiments, the carbohydrate domain binds to the glycan of the vesicular stomatitis virus glycoprotein, the envelope glycoprotein GP120, or both.

In some embodiments, the target is a free protein. The free protein includes a carbohydrate. A free protein is a protein that is not bound to a surface; bound to and/or partially encapsulated in shell or layer; or both. A free protein is not a part of a viral vector or virus. For example, a free protein is not bound to and/or partially encapsulated in a capsid or envelope of a viral vector of virus. Examples of free proteins are antibodies and fragments thereof, fetuin, and other glycosylated proteins that are not tethered to a surface of a shell.

In some embodiments, the target molecule is an antibody or a fragment thereof that includes a carbohydrate group. The antibody may be any type of antibody (e.g., IgG, IgM, IgA, IgE, IgD) or a fragment thereof (e.g., Fab, scFv, Nb, Fc fusion, BsFab, Nb-scFV fusion, multispecific Nb tandem, and the like) and produced in any source (e.g., humans, various cell lines, camelids, and the like). The antibody includes at least on conjugated monosaccharide, disaccharide, or glycan. The monosaccharide may be any monosaccharide as described herein. The glycan and/or the disaccharide may include any glycosylation pattern; that is, any pattern of monosaccharides, each monosaccharide linked to at least one other monosaccharide through a glycosidic linkage. The monosaccharides included in a disaccharide or a glycan of the target antibody may be any monosaccharide as described herein including stereoisomers thereof, and ionized versions thereof, as well as have any substituent or modification as described herein. Additionally, each glycosidic linkage in a glycan affinity group may be of any configuration as described herein.

In some embodiments, the target molecule is a free protein (e.g., an antibody or fragment thereof) that includes a carbohydrate. In some embodiments, the carbohydrate is a monosaccharide, disaccharide, or glycan. In some embodiments, the carbohydrate includes a sialic acid (e.g., Neu5Ac). In some embodiments, the sialic acid is attached to an adjacent saccharide in an α2,3 linkage (e.g., α2,3-Neu5Ac). In some embodiments, the sialic acid is attached to an adjacent saccharide in a an α2,6 linkage (e.g., α2,6-Neu5Ac). In some embodiments, the target molecule includes multiple carbohydrates. In some such embodiments, the carbohydrates have the same identity. In other such embodiments, the carbohydrates have different identities. In some embodiments, the target molecule is a protein that includes at least one carbohydrate that includes an α2,6 linked sialic acid (e.g., α2,6-Neu5Ac) and at least one carbohydrate that includes an α2,3 linked sialic acid (e.g., α2,3-Neu5Ac). In some embodiments the target molecule is fetuin, a protein that includes multiple sialic acid moieties. In some embodiments the target molecule is mucin or a fragment thereof. In some embodiments the target molecule is mucin of a fragment thereof. In some embodiments the target molecule is asialofetuin of a fragment thereof.

In some embodiments, the antibody or fragment thereof includes a carbohydrate that includes a fucose group. In some embodiments, the fucose group is alpha fucose (αfucose α-fucose, alpha-fucose). In some embodiments, the antibody or fragment thereof includes a carbohydrate that includes a sialic acid group (e.g., Neu5Ac). In some embodiments, the sialic acid is attached to an adjacent saccharide in an α2,3 linkage (e.g., α2,3-Neu5Ac). In some embodiments, the sialic acid is attached to an adjacent saccharide in a an α2,6 linkage (e.g., α2,6-Neu5Ac). In some embodiments, the antibody or fragment thereof includes multiple carbohydrates. In some such embodiments, the carbohydrates have the same identity. In other such embodiments, the carbohydrates have different identities. In some embodiments, the target molecule is an antibody or fragment thereof that includes at least one carbohydrate that includes an α2,6 linked sialic acid (e.g., α2,6-Neu5Ac) and at least one carbohydrate that includes an α2,3 linked sialic acid (e.g., α2,3-Neu5Ac).

In some embodiments the target molecule is fetuin, a protein that includes multiple sialic acid moieties.

In some embodiments, the carbohydrate binding domain includes the carbohydrate binding domain of a lectin. Lectins are a class of carbohydrate binding proteins that bind specific free monosaccharide/disaccharide, or monosaccharide/disaccharide, of a glycan; glycosylation patterns; or both, for example, on glycosylated biomolecules. The carbohydrate binding domain of a lectin is the minimum portion of the lectin required to bind to the appropriate monosaccharide/disaccharide or glycosylation pattern. In some embodiments, the lectin carbohydrate binding domain has been engineered to increase the binding affinity and/or specificity to one or more monosaccharide/disaccharide of a glycan or glycosylation patterns. In some embodiments, the carbohydrate binding domain includes the entire lectin protein or an engineered form thereof.

There are a wide variety of lectin types derived from different plant species with different binding specificities as well as varying molecular weights and isoelectric points Examples of lectin proteins are shown in Table 4. The ability of some lectins to bind the intended target is influenced by the presence or lack thereof of a metal ion (Table 4). Table 5 shows the specific glycosylation pattern or monosaccharide bound by each lectin (Sugar Specificity) as well as molecules such as monosaccharide, disaccharides, and/or glycans that inhibit or reduce binding of the lectin to the intended target (Inhibitor or Eluting Sugar). In some embodiments, the carbohydrate binding domain includes the carbohydrate binding domain or an engineered version thereof of any one of the lectins in Table 4 or Table 5. In some embodiments, the carbohydrate binding domain includes any one of the lectins or an engineered version thereof in Table 3 or Table 4. In some embodiments, the carbohydrate binding domain comprises the carbohydrate binding domain, the entire protein, or an engineered version thereof of Concanavalin A, Galanthus nivalis, or Musa paradisiaca. The carbohydrate binding domains may be produced recombinantly using methods known in the art.

TABLE 4
Example Lectins.

| | Common | | Metal Ions |
|---|---|---|---|
| Lectin | Name | Source | Present |
| Agaricus bisporus | ABL | Agaricus bisporus | No |
| | | (white button mushrooms) | |
| Aleuria aurantia | AAL | Aleuria aurantia mushrooms | — |
| Bauhinia purpurea | BPL, BPA | Bauhinia purpurea alba | No |
| | | (camel's foot tree) seeds | |
| Concanavalin A | Con A | Canavalia ensiformis | Ca++, Mn++ |
| | | (jack bean) seeds | |
| Succinylated | sCon A | — | — |
| Concanavalin A | | | |
| Datura stramonium | DSL | Datura stramonium (thorn apple, | No |
| | | jimson weed) seeds | |
| Dolichos biflorus | DBA | Dolicos biflorus | Ca++, Mn++, |
| | | (horse gram) seeds | Mg ++, Zn++ |
| Erythrina cristagalli | ECL, ECA | Erythrina cristagalli | Ca++, Mn++, |
| | | (coral tree) seeds | Zn++ |
| Galanthus nivalis | GNL | Galanthus nivalis | No |
| | | (snowdrop) bulbs | |
| Griffonia (Bandeiraea) | GSL I, | Griffonia (Bandeiraea) | Ca++, Mn++ |
| simplicifolia I | BSL I | simplicifolia seeds | |

simplicifolia I Isolectin

| | | | |
|---|---|---|---|
| Griffonia (Bandeiraea) | GSL I - B4 | Griffonia (Bandeiraea) | Ca++, Mn++ |
| simplicifolia | | simplicifolia seeds | |
| I Isolectin | | | |
| B4 | | | |
| Griffonia (Bandeiraea) | GSL II, | Griffonia (Bandeiraea) | Ca++, Mn++ |
| simplicifolia II | BSL II | simplicifolia seeds | |
| Hippeastrum hybrid | HHL, AL | Hippeastrum hybrid (amaryllis) | No |
| | | bulbs | |
| Jacalin | Jacalin | Artocarpus integrifolia | No |
| | | (jackfruit) seeds | |
| Lens culinaris | LCA, LcH | Lens culinaris (lentil) seeds | Ca++, Mn++ |
| Lotus tetragonolobus | LTL | Lotus tetragonolobus, | Ca++, Mn++ |
| | | Tetragonolobus purpurea | |
| | | (winged pea, asparagus pea) | |
| | | seeds | |
| Lycopersicon esculentum | LEL, TL | Lycopersicon esculentum | -- |
| | | (tomato) fruit | |
| Maackia amurensis I | MAL I, | Maackia amurensis seeds | No |
| | MAL | | |
| Maackia amurensis II | MAL II, | Maackia amurensis seeds | No |
| | MAH | | |
| Maclura Pomifera | MPL | Maclura pomifera (Osage | No |
| | | orange) seeds | |
| Musa paradisiaca | BanLec | Musa paradisiaca (banana fruit) | |
| Narcissus pseudonarcissus | NPL, NPA, | Narcissus pseudonarcissus | No |
| | DL | (daffodil) bulbs | |
| Peanut | PNA | Arachis hypogaea peanuts | Ca++, Mg++ |
| Phaseolus vulgaris | PHA-E | Phaseolus vulgaris (red kidney | Ca++, Mn++ |
| Erythroagglutinin (PHA- | | bean) seeds | |
| E) | | | |
| Phaseolus vulgaris | PHA-L | Phaseolus vulgaris (red kidney | Ca++, Mn++ |
| Leucoagglutinin (PHA-L) | | bean) seeds | |
| Pisum sativum | PSA | Pisum sativum (pea) seeds | Ca++, Mn++ |
| Ricinus communis I | RCA I, | Ricinus communis (castor bean) | No |
| | RCA120 | seeds | |
| Ricin A Chain | Ricin A | RCA60 | No |
| | Chain | | |
| Ricin B Chain | Ricin B | RCA60 | No |
| | Chain | | |
| Sambucus nigra | SNA, EBL | Sambucus nigra (elderberry) | No |
| | | bark | |
| Solanum tuberosum | STL, PL | Solanum tuberosum (potato) | No |
| | | tubers | |
| Soybean | SBA | Glycine max (soybean) seeds | Ca++, Mn++ |
| Ulex europaeus I | UEA I | Ulex europaeus (furze gorse) | Ca++, Mn++, |
| | | seeds | Zn++ |
| Vicia villosa | VVL, VVA | Vicia villosa (hairy vetch) seeds | Ca++, Mn++ |
| Wheat Germ | WGA | Triticum vulgaris (wheat germ) | Ca++ |
| Succinylated Wheat Germ | Succinylated | Triticum vulgaris (wheat germ) | Ca++ |
| | WGA | | |
| Wisteria floribunda | WFA, | Wisteria floribunda (Japanese | — |
| | WFL | wisteria) seeds | |

TABLE 5
Example lectins and their glycosylation pattern specificity and inhibitors

| | | | Inhibitor | Inhibitor |
|---|---|---|---|---|
| | Sugar | Sugar | or Eluting | or Eluting |
| Lectin | Specificity¹ | specificity² | Sugar¹ | Sugar² |
| Agaricus bisporus | Gal(β-1,3) GalNAc | O-linked Gal β-1,3 | Fetuin | Gal |
| | | GalNAc | | |
| Aleuria aurantia | Fucα6GlcNAc | Fuc | L-Fuc | Fuc |
| Bauhinia purpurea | Galβ3GalNAc | Primarily Gal β-1,3 or | Lactose | Gal |
| | | 1,4 but will also bind β | | |
| | | GalNAc more weakly | | |
| Concanavalin A | aMan, aGlc | Branched and terminal | MeαMan + | Man |
| | | mannose [High-Man, | MeαGlc | |
| | | Mana-1,6 (Mana-1,3) | | |
| Succinylated | — | αMan, αGlc | — | Man |
| Concanavalin A | | | | |
| Datura | (GlcNAc)2-4 | GlcNAc β-1,4; GlcNAc | Chitin | Lac |
| stramonium | | oligomers and LacNAc | hydrolysate | |
| | | (Gal β-1,4GlcNAc) | | |
| Dolichos biflorus | αGalNAc | GlcNAc β-1,4; GlcNAc | GalNAc | Gal |
| | | oligomers and LacNAc | | |
| | | (Gal β-1,4GlcNAc) | | |
| Erythrina | Galβ4GlcNAc | Gal β-1,4 GalNAc | Lactose | Gal |
| cristagalli | | | | |
| Galanthus nivalis | αMan | Terminal a-1,3 | MeαMan | Man |
| | | mannose | | |
| Griffonia | αGal, αGalNAc | a-Galactose, also binds | Gal/GalNAc | Gal |
| (Bandeiraea) | | some GalNAc | | |
| simplicifolia I | | | | |
| Griffonia | αGal | αGal | Gal or | Gal |
| (Bandeiraea) | | | Raffinose | |
| simplicifolia I | | | | |
| Isolectin B4 | | | | |
| Griffonia | α or βGIcNAc | Terminal GlcNAc | Chitin | GalcNAc |
| (Bandeiraea) | | | hydrolysate | |
| simplicifolia II | | | or GlcNAc | |
| Hippeastrum | αMan | a-1,3 Mannose and a- | MeaMan | Man |
| hybrid | | 1,6 mannose | | |
| Jacalin | Galβ3GalNAc | Gal β-1,3 GalNAc | Gal or | Gal |
| | | | Melibiose | |
| Lens culinaris | αMan, αGlc | Complex (man/GlcNAc | MeαMan + | Man |
| | | core with a-1,6 Fuc) | MeαGlc | |
| Lotus | αFuc | Terminal a-Fuc, Lewis | L-Fuc | Fuc |
| tetragonolobus | | x | | |
| Lycopersicon | (GlcNAc)2-4 | β-1,4 GlcNAc | Chitin | GlcNAc |
| esculentum | | oligomers | hydrolysate | |
| Maackia | Galβ4GlcNAc | Galactosyl β-1,4 N- | Lactose | Lac |
| amurensis I | | acetylglucosamine; a- | | |
| | | 2,3 sialic acid | | |
| Maackia | Neu5Acα3Galβ3GalNAc | a-2,3 sialic acid- | Human | Lac |
| amurensis II | | LacNAc structure | Glycophorin | |
| Maclura Pomifera | Galβ3GalNAc | Gal β-1,3 GalNAc; | Gal | Gal |
| | | GalNAc | | |
| Musa paradisiaca | αMan, αGlc | αMan, αGlc | MeαMan | Man |
| Narcissus | βMan | Terminal and internal | MeαMan | Man |
| pseudonarcissus | | Man | | |
| Peanut | Galβ3GalNAc | Terminal Gal (β-OR) | Gal | Gal |
| Phaseolus vulgaris | Galβ4GlcNAcβ2Manα6 | Complex-type N- | bovine | Lac |
| Erythroagglutinin | (GlcNAcβ4) | glycans with outer Gal | thyro- | |
| (PHA-E) | (GlcNAcβ4Manα3) | and bisectng GlcNAc | globulin, | |
| | Manβ4 | | acetic acid | |
| Phaseolus vulgaris | Galβ4GlcNAcβ6 | β-1,6 Brandched | bovine | Gal |
| Leucoagglutinin | (GlcNAcβ2Manα3) | trimannosyl core N- | thyro- | |
| (PHA-L) | Manα3 | linked glycans | globulin, | |
| | | | acetic acid | |
| Pisum sativum | αMan, αGlc | Man, (Fuc a-1,6 | MeαMan + | Man |
| | | GlcNAc, a-D-Glc, a-D- | MeαGlc | |
| | | Man) | | |
| Ricinus communis | Gal | Gal | Gal or | Lac, Gal |
| I | | | Lactose | |
| Ricin A Chain | — | — | — | |
| Ricin B Chain | Gal | β-Gal/GalNAc | Gal or | Lac |
| | | | Lactose | |
| Sambucus nigra | Neu5Acα6Gal/GalNAc | a-2,6 sialic acid- | Lactose in | Lac |
| | | LacNAc structure | buffered | |
| | | | saline and | |
| | | | acetic acid | |
| Solanum | (GlcNAc)2-4 | GlcNAc oligomers, | Chitin | GlcNAc |
| tuberosum | | LacNAc | hydrolysate | |
| Soybean | α > βGalNAc | a- or β -linked terminal | GalNAc | Gal |
| | | GaINAc, GalNAc a-1,3 | | |
| | | Gal | | |
| Ulex europaeus I | αFuc | αFuc | L-Fuc | Fuc |
| Vicia villosa | GalNAc | GaINAc a, Tn antigen | GalNAc | GalNAc |
| Wheat Germ | GlcNAc | β-GlcNAc, sialic acid, | Chitin | GlcNac |
| | | GalNAc | hydrolysate | |
| | | | or GlcNAc | |
| | | | with acid or | |
| | | | salt | |
| Succinylated | GlcNAc | GlcNAc | Chitin | GlcNac |
| Wheat Germ | | | hydrolysate | |
| | | | or GlcNAc | |
| | | | with acid or | |
| | | | salt | |
| Wisteria | GalNAc | GalNAc | GalNAc, | Gal |
| floribunda | | | acetic acid | |

¹Lectin Application and Resource Guide, Vector Laboratories, Inc.
²Essentials of Glycobiology [Internet]. 4th edition. Varki A, Cummings RD, Esko JD, et al., editors. Cold Spring Harbor (NY): Cold Spring Harbor Laboratory Press; 2022.
Fuc = L-fucose; Gal = D-galactose; GalNAc = N-acetylgalactosamine; Gcl = D-glucose; GlcNAc = N-acetylglucosamine; Man = mannose; MeaGlc = a-methylglucoside; MeaMan = a-methylmannoside; Neu5Ac = N-acetylneuraminic acid (sialic acid); SA = sialic acid.

In some embodiments, the affinity group includes a carbohydrate binding ligand capable of binding to a carbohydrate of a target molecule. The carbohydrate binding ligand can be chosen based on the target molecule to be isolated. In some embodiments, the carbohydrate binding ligand binds to the glycan of the vesicular stomatitis virus glycoprotein, the envelope glycoprotein GP120, or both. In some embodiments, the carbohydrate binding ligand binds to a carbohydrate of a target antibody. Suitable carbohydrate binding ligands include ligands known to bind to carbohydrates, glycosylated viral vector surface proteins, inhibit viruses, or have a chemical structure similar to those reported. In some embodiments, the carbohydrate binding ligand includes thiosalicylic acid (TSA; formula 1 below); 4-mercaptophenyl boronic acid (MPBA; 2); 4-mercaptophenol (MPhOH; 3); gallic acid (4); serotonin (5); epigallocatechin gallate (EGCG; 6); reactive red 120 (7), cibracon brilliant yellow 3G-P (8); reactive blue 4 (9); or any combination thereof (the structures are shown below). When such molecules are an affinity groups, the separation group displayed on the surface of the support substrate includes the reaction product between a reactive handle on the affinity groups and a corresponding reactive handle (as discussed herein).

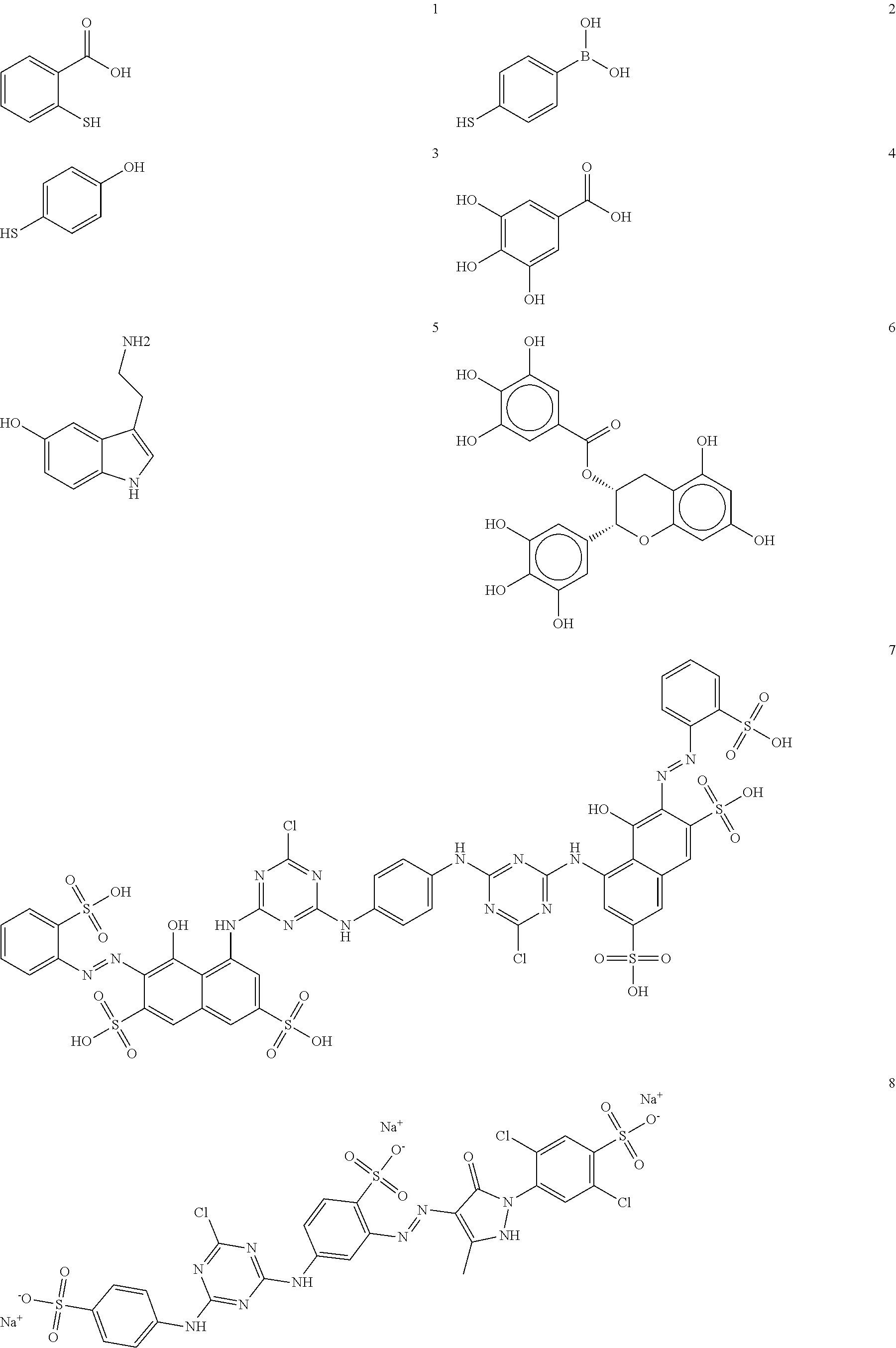

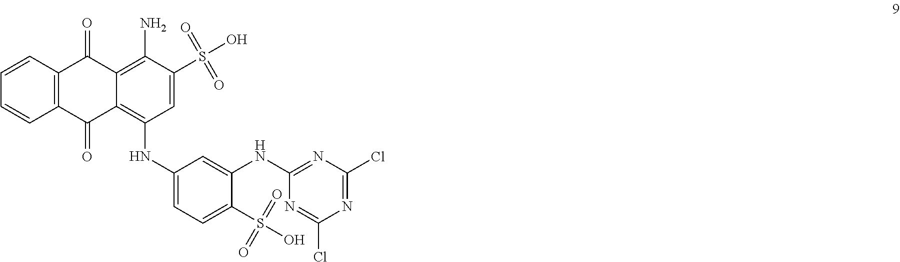 [SMILES: Nc1c(S(=O)(=O)O)cc(Nc2ccc(S(=O)(=O)O)c(Nc3nc(Cl)nc(Cl)n3)c2)c2c1C(=O)c1ccccc1C2=O]

In some embodiments, the affinity group includes

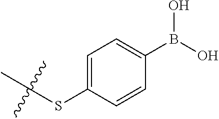 [SMILES: *Sc1ccc(B(O)O)cc1]

where S may or may not be a part of a reaction product.

In some embodiments, the separation media includes a plurality of separation ligands that include a separation group that is an assistance group. An assistance group is a chemical moiety that facilitates the binding of the target molecule to the affinity group; binds the target molecule through electrostatic interactions and/or hydrophobic interactions; or both. In some embodiments, the assistance group may allow for a high density of target molecules to bind to separation ligands that include an affinity group. In some embodiments, the assistance group may aid in attracting the target molecule to the support substrate such as to allow for the target molecule to be in proximity to a separation group that includes an affinity group. For example, the assistance group may be ionizable or possesses a formal charge which may be opposite the charge of the target molecule. In such cases, the oppositely charged assistance group may attract the target molecule to the support substrate which may allow the target molecule to bind to the affinity group.

In some embodiments, the assistance group functions as a cation or anion exchange chromatography ligand. Anion exchange ligands have a positively charged functional group that targets negatively charged target molecules through electrostatic interactions. The anion exchange ligand may possess a formal positive charge, or the positive charge can be induced through the pH of the solution that the anion exchange ligand is exposed to. Cation exchange ligands have a positively charged functional groups that target negatively charged target molecules through electrostatic interactions. The anion exchange ligand may possess a formal negative charge, or the negative charge can be induced through the pH of the solution that the anion exchange ligand is exposed to.

In some embodiments, the assistance group possesses a positive formal charge or is ionizable under certain pH conditions to have a positive charge. Such assistance groups may be beneficial when the target molecule has a negative formal charge. Examples of such assistance ligands include primary, secondary, tertiary, and quaternary amines. Suitable amines may be diamines, triamines, and polyamines.

Examples of primary amines include methylene diamine, ethylene diamine, propylene diamine, butylenediamine (putrescine), pentylamine, or any aliphatic diamine with 1-18 carbons between the terminal amines, covalently attached via one of the amines. Such ligands can be made from polyamines such as ethylene diamine, diethylenetriamine, triethylenetetramine covalently attached via one of the amines.

Examples of secondary amines can include any of the aforementioned primary amines immobilized to the substrate, substituted with an additional R-group as described above. In cases in which diamines are used, secondary amines may also be formed by covalent interaction with the substrate coupling both amines to the substrate. Ligands containing secondary amines with the structure of the ligand may also be immobilized such as linear polyethyleneimine, spermidine, or spermine. Furthermore, groups containing a non-terminal primary amine (e.g., 3-aminopentane) may also be conjugated to the substrate to result in a secondary amine.

Examples of suitable tertiary amines include N,N-dimethylethylenediamine; N,N-dimethylpropylenediamine; N,N-diethylpropylenediamine; or any aliphatic diamine with aliphatic carbon group substitution on one or both amines ranging from one to six carbons, with an linker having 2-18 carbons between the terminal amines.

Examples of quaternary amines include any of the aforementioned primary amines that have undergone a quaternarization reaction resulting in a permanent positive charge. Such reactions can be performed with alkyl groups such as methyl iodide or aryl groups such as benzyl iodide. Quaternary amines can further include any of the aforementioned tertiary amines that have undergone a quaternarization reaction resulting in a permanent positive charge. Such reactions can be described by the Menshutkin reaction which uses an alkyl halide to form a quaternary ammonium salt from a reaction with a tertiary amine. Such reactions can be performed with alkyl containing groups of varying length such as butyl bromide or aryl groups such as benzyl chloride or combinations therein. Additionally, compounds containing quaternary amines can be immobilized directly.

In other embodiments, the assistance group possesses a negative formal charge or is ionizable under certain pH conditions to have a negative charge. Such assistance group may be beneficial when the target molecule has a positive formal charge. The difference in charge of target molecule and the assistance molecule may allow for an electrostatic interaction between the target molecule and the assistance group thereby allowing the target molecule to be proximate to the support surface and the affinity groups which may increase the probability of the target molecule of binding to an affinity group.

In some embodiments, the assistance group is such that it is able to induce hydrophobic interactions with the target molecules. Hydrophobic interactions exploit the differences in hydrophobicity of between the target molecules and possible impurities. In one embodiment, such ligands include aliphatic chains with three carbons or longer (common used lengths include butyl, pentyl, hexyl, heptyl, octyl, nonyl, decyl, undecyl, and dodecyl), benzyl, phenyl, phenol, pyridine, boronic acid groups, branched polymers such as polypropylene glycol, and sulfur-containing thiophilic ligands such as propanethiol, 2-butanethiol, 3,6-dioxa-1,8-octanedithiol, octanethiol, benzyl mercaptan, 2-mercaptopyridine, thiophenol, 1,2-ethanedithiol, 1,4-benzenedimethanethiol, 2-phenylethanethiol, and the like, and any combination thereof.

In some embodiments, separation ligands that include an assistance group can be directly incorporated into a functionalized layer of a support substrate through polymerization of a monomer that includes an assistance group.

In some embodiments, the separation media includes a plurality of separation ligands that includes a separation group that is a capping group. A capping group is a chemical moiety that prevents reactive groups of the support membrane from reacting with the target molecule or any other molecule in the isolation solution. A capping group may be employed to block support substrate reactive handles that have not reacted with other separation ligands. A capping group may be used to cap the end of a polymer chain. Capping groups may be any chemical group that is non-reactive towards the target molecule or other molecules the isolation solution.

In some embodiments, a separation ligand immobilized on a support substrate has the formula (SLim)

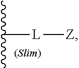

where L is a linker, Z is a separation group, and the vertical black line is the support substrate.

Each separation ligand of the plurality of separation ligands has the formula SL

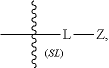

where L is a linker and Z is a separation group. L separates the support substrate from Z. The separation group may include an affinity group, capping group, or assistance group. The affinity group may be any affinity group as disclosed herein. The capping group may be any capping group as disclosed herein. The assistance group may be any capping group as disclosed herein.

Separation ligands of multiple chemical compositions may be immobilized to a single support substrate. For example, a support substrate may include a first portion of a separation ligands of formula SL and a second portion of separation ligands of formula SL. In some embodiments, the first portion and the second portion of separation ligands include the same affinity group but have different linkers (L). In other embodiments, the first portion and the second portion of the separation ligands may have the same linker but have different separation group.

In some embodiments, L is of formula L1 such that the separation ligand of formula SL is of formula SL1.

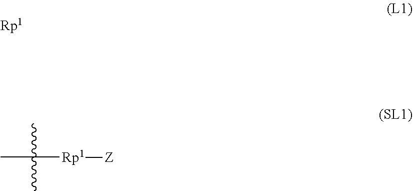

Rp¹ is a reaction product, and Z is the separation group. A reaction product is the chemical group resulting from the reaction of two cooperative functional handles (as discussed herein). In a separation ligand of formula SL1, the linker is the reaction product. The reaction product Rp¹ links the support substrate (not shown) and the separation group (Z). A covalent bond from Rp¹ to the support substrate is the point of covalent attachment of the linker (L1) to the support substrate. A covalent bond from Rp¹ to the separation group (Z) is the point of covalent attachment of the linker (L1) to the separation group (Z). Rp¹ may be any reaction product as disclosed herein. The reaction product (Rp¹) may be the reaction product between any two cooperative reactive handles (as described herein). Examples of reaction products include amides, ureas, thioureas, carbamates, carbonates, esters, thioethers, ethers, and triazoles. In some embodiments, a reaction product (e.g., such as Rp¹) is Rp^{A}, Rp^{B}, Rp^{C}, Rp^{D}, Rp^{E}, Rp^{F}, Rp^{G}, Rp^{H}, Rp^{I}, Rp^{J}, Rp^{K}, Rp^{L}, Rp^{M}, or an isomer thereof. Chemical structures of Rp^{A}-Rp^{M} are depicted below.

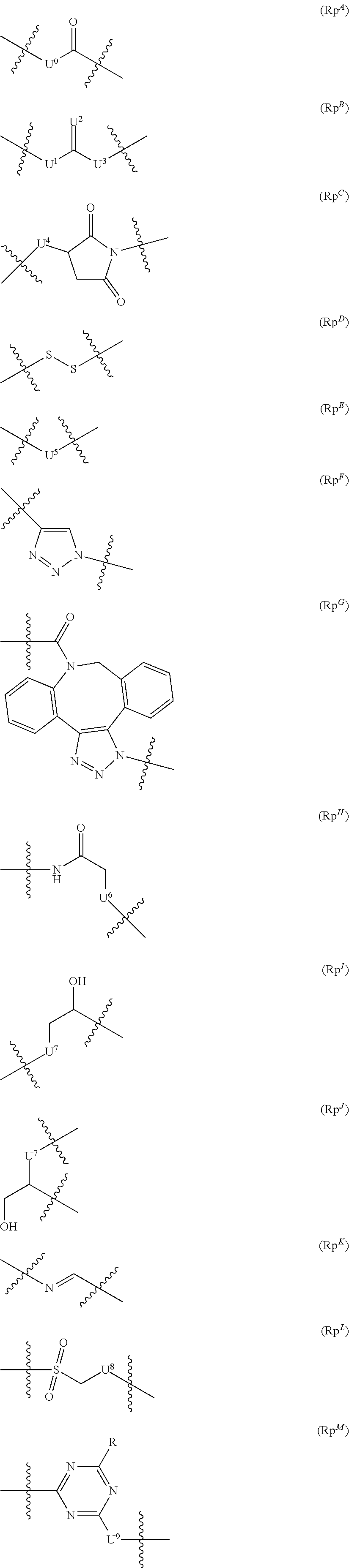

where U⁰, U⁴, U⁵, U⁶, U⁷, U⁸ and U⁹ are independently NH, N, O, or S. For Rp^{B} each of U¹, U², and U³ are independently NH, N, O, or S. R in Rp^{M} may be H, an organic group, or a halogen. The reaction products have two connection points, each of which may be covalently linked to the support substrate or any component of a separation ligand. For separation ligands of formula SL1, one connection point the reaction product Rp¹ is linked to the separation group while the other connection point of the reaction product Rp¹ is linked to the support substrate.

In some embodiments where the separation ligand is of formula SL1, Rp¹ is Rp^{A} where U⁰ is NH. In some such embodiments, the amide nitrogen (U⁰) of Rp^{A} is covalently linked to the separation group. In other such embodiments, the amide nitrogen of Rp^{A} is covalently linked to the support substrate.

In some embodiments where the separation ligand is of formula SL1, Rp¹ is Rp^{A} where U⁰ is O. In some such embodiments, the ester oxygen (U⁰) of Rp^{A} is covalently linked to the separation group. In other such embodiments, the ester oxygen of Rp^{A} is covalently linked to the support substrate.

In some embodiments where the separation ligand is of formula SL1, Rp¹ is Rp^{L} where U⁸ is O. In some such embodiments, oxygen is covalently linked to the separation group. In other such embodiments, the oxygen is covalently linked to the support substrate. In some embodiments when the affinity group includes thiosalicylic acid; 4-mercaptophenyl boronic acid; 4-mercaptophenol; gallic acid; serotonin; epigallocatechin gallate (EGCG; 6); reactive red 120, cibracon brilliant yellow 3G-P; or reactive blue 4, the separation ligand of SL1 includes Rp^{L}.

In some embodiments where the separation ligand is of formula SL1, Rp¹ is Rp^{M} where U⁹ is O and R is a halogen. In some such embodiments, the oxygen is covalently linked to the separation group. In other such embodiments, the oxygen is covalently linked to the support substrate. In some embodiments when the affinity group includes thiosalicylic acid; 4-mercaptophenyl boronic acid; 4-mercaptophenol; gallic acid; serotonin; epigallocatechin gallate (EGCG; 6); reactive red 120, cibracon brilliant yellow 3G-P; or reactive blue 4, the separation ligand of SL1 includes Rp^{M}.

The identity of a reaction product (e.g., Rp¹) depends at least in part on the type of conjugation chemistry used to form the reaction product. In a conjugation reaction, each component being linked together includes a reactive handle, such that the reactive handles are cooperative reactive handles. Components that include a reactive handle for conjugation reactions are termed precursor compounds or precursors. A precursor compound includes the component and a reactive handle covalently linked to the component. Cooperative handles or cooperative reactive handles are two or more reactive handles that when exposed to each other under favorable reaction conditions a conjugation reaction occurs to form a reaction product between the reactive handles. Components that have been conjugated through a conjugation reaction may be referred to as a conjugate. For example, component A and component B are to be conjugated through a conjugation reaction. The component A precursor includes a reactive handle X. The component B precursor includes a reactive handle Y. X and Y are cooperative. A conjugation reaction between the component A precursor and the component B precursor results in the formation of an A-B conjugate that includes the reaction product between X and Y. It is understood that the notation of a conjugate is from the perspective of the conjugated components, not the precursors of those components (i.e., A-B conjugate not A precursor-B precursor conjugate). This is because upon completion of the conjugation reaction, the precursor components are no longer precursors. In the case of a component precursor that includes two independently reactive handles, one of which has been reacted with a different component precursor to form a conjugate, the conjugate notation is still from the perspective of the conjugated components, not the precursor components, with the understanding that the conjugate includes the unreacted second reactive handle. For example, a component D precursor includes a first reactive handle J and a second reactive handle Z. The component B precursor has the reactive handle Y. J and Y are cooperative handles. A conjugation reaction between the component A precursor and the component B precursor results in the formation of an A-B conjugate that includes the reaction product between J and Y. The A-B conjugate also includes the unreacted second reactive handle Z.

Any pair of cooperative reactive handles may be used to forma reaction product of the present disclosure. Examples of cooperative handles include an activated ester and an amine; an amine and an NHS-ester; a hydroxyl and an NHS-ester; a hydroxyl and an epoxide; an acyl chloride and an amine; and acyl chloride and a alcohol; an amine and an epoxide; a thiol and an epoxide; a thiol and a maleimide; a disulfide and a thiol; an azide and an alkyne (azide and a linear alkyne in the presence of Cu(I); an azide and a cyclic alkyne such as cyclooctyne, difluorinated cyclooctyne, dibenzocyclooctyne, TMTH-SulfoxImine, biarylazacyclooctynone, or bicyclo[6.1.0]nonyne); an amine and an isocyanate; an amine and an isothiocyanate, a amine and a benzoyl fluoride; a thiol and a iodoacetamide; a thiol and a bromoacetamide; a disulfide and 2-thiopyridine; a thiol and 3-arylpropiolonitirle; a phenol and a diazonium salt; a phenol and 4-phenyl-1,2,4-triazoline-3,5-dione; a phenol and aldehyde, and a aniline; a hydroxyl and sodium periodate; a thiol and an iodoacetamide; an amine and a pyridoxal phosphate; an azide and a functionalized triphenyl phosphine; a tetrazine and a strained alkene; and the like.

Examples of individual reactive handles that may be used to form the separation media of the present disclosure include Rh^{A} (hydroxyl), Rh^{B} (thiol), Rh^{C} (amine), Rh^{D} (activated ester), Rh^{E} (azide), Rh^{F} (alkyne), Rh^{G} (NHS-ester), Rh^{H} (maleimide), Rh^{I} (where X is a Cl, Br, or I leaving group attached to carbon that can undergo nucleophilic substitution; e.g., a bromoacetamide or iodoacetamide), Rh^{J} (cyclooctyne), Rh^{K} (isocyanate), Rh^{L} (isothiocyanate), Rh^{M} (where X is a Cl, Br, or I leaving group attached to carbon that can undergo nucleophilic substitution), Rh^{N} (an epoxide), Rh^{O} (an acyl chloride), R^{Q} (halotriazine where X is Cl, I, or Br and R is an organic group, H or a halogen), Rh^{R} (vinyl sulfone), and isomers thereof. Chemical structures of Rh^{A}—Rh^{R} are depicted below.

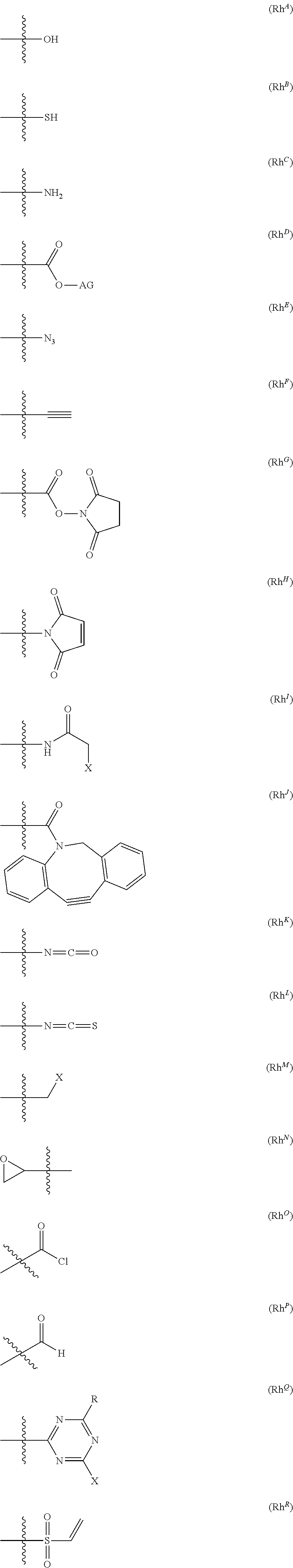

where X in Rh^{M} and Rh^{I} may be -chloro, -bromo, or -iodo.

Rh^{D} is an activated ester where AG is an activating group. An activated ester is an ester that is reactive with an activated ester cooperative reaction handle (e.g., an amide) in a conjugation reaction. Activated esters may be denoted as the type of activated ester or by the activating group. Examples of activating groups include O-acylisoureas, benzotriazoles (with a bond between the ester oxygen and one nitrogen of the triazole), and pentafluorophenyl. In some embodiments, Rh^{D} may be an activated ester of a carboxylic acid. In such embodiments, the activated ester is formed through the reaction of a carboxylic acid with one or more reagents that install the activating group. For example, a carboxylic acid may be converted into an activated ester having a O-acylisoureas activating group by treating the carboxylic acid with various carbodiimide reagents (e.g., N,N′-Dicyclohexylcarbodiimide, 1-Ethyl-3-(3 dimethylaminopropyl)carbodiimide, diisopropylcarbodiimide (DIC)) under favorable reaction conditions. A carboxylic acid may be converted into an activated ester having a benzotriazole activating group by treating the carboxylic acid with various carbodiimide reagents followed by treatment with hydroxybenzotriazole (HOBT) or by treating the carboxylic acid with various benzotriazole containing compounds (e.g., O-(Benzotriazol-1-yl)-N,N,N′,N′-tetramethyluronium hexafluorophosphate (HBTU); O-(Benzotriazol-1-yl)-N,N,N′,N′-tetramethyluronium tetrafluoroborate (TBTU); 2-(1H-benzotriazol-1-yl)-1,1,3,3-tetramethyluronium hexafluorophosphate (I-BTU); Benzotriazol-1-yloxy)tris(dimethylamino)phosphonium hexafluorophosphate (BOP); (Benzotriazol-1-yloxy)tripyrrolidinophosphonium hexafluorophosphate (PyBOP); and O-(7-Azabenzotriazol-1-yl)-N,N,N′,N′-tetramethyluronium tetrafluoroborate (TATU) under favorable reaction conditions. Other reagents are available for making activated esters from carboxylic acids including bromotripyrrolidinophosphonium hexafluorophosphate (PyBrOP); O—(N-Succinimidyl)-1,1,3,3-tetramethyl-uronium tetrafluoroborate (TSTU); O-(5-Norbornene-2,3-dicarboximido)-N,N,N′,N′-tetramethyluronium tetrafluoroborate (TNTU); O-(1,2-Dihydro-2-oxo-1-pyridyl-N,N,N′,N′-tetramethyluronium tetrafluoroborate (TPTU); and 3-(Diethylphosphoryloxy)-1,2,3-benzotriazin-4(3H)-one (DEPBT); carbonyldiimidazole. In some embodiments, the activated ester may be created in situ from a carboxylic acid and not isolated prior to a conjugation reaction.

Rh^{O} is an acyl chloride. Acyl chlorides may be prepared from carboxylic acids, for example, using thionyl chloride. Acyl chlorides may not be stable and as such, may be prepared in situ and not isolated prior to a conjugation reaction.

Reactive handles Rh^{A}, Rh^{B}, Rh^{C}, Rh^{D}, Rh^{E}, Rh^{F}, Rh^{G}, Rh^{H}, Rh^{I}, Rh^{J}, Rh^{K}, Rh^{L}, Rh^{M}, Rh^{N}, Rh^{O}, Rh^{P}, Rh^{Q}, Rh^{R} include various pairs of cooperative handles that can from the reaction products of Rp^{A}, Rp^{B}, Rp^{C}, Rp^{D}, Rp^{E}, Rp^{F}, Rp^{G}, Rp^{H}, Rp^{I}, Rp^{J}, Rp^{K}, Rp^{L}, and Rp^{M}. For example, under favorable reaction conditions, a conjugation reaction between Rh^{A} and Rh^{D} forms Rp^{A} where U⁰ is O. Under favorable reaction conditions, a conjugation reaction between Rh^{D} and Rh^{C} forms Rp^{A} where U⁰ is NH. Under favorable reaction conditions, a conjugation reaction between Rh^{C} and Rh^{G} forms Rp^{A} where U⁰ is NH. Under favorable reaction conditions, a conjugation reaction between Rh^{B} and Rh^{H} forms Rp^{C} where U⁴ is S. Under favorable reaction conditions, a conjugation reaction between two Rh^{B} forms Rp^{D}. Under favorable reaction conditions, a conjugation reaction between Rh^{C} and Rh^{I} forms Rp^{H} where U⁶ is NH. Under favorable reaction conditions, a conjugation reaction between Rh^{B} and Rh^{I} forms Rp^{H} where U⁶ is S. Under favorable reaction conditions, a conjugation reaction between Rh^{M} and Rh^{B} forms Rp^{E} where U⁵ is S. Under favorable reaction conditions, a conjugation reaction between Rh^{M} and Rh^{C} forms Rp^{E} where U⁵ is NH. Under favorable reaction conditions, a conjugation reaction between Rh^{K} and Rh^{C} forms Rp^{B} where U¹ and U³ are NH and U^{z} is O. Under favorable reaction conditions, a conjugation reaction between Rh^{L} and Rh^{C} forms Rp^{B} where U¹ and U³ are NH and U² is S. Under favorable reaction conditions, a conjugation reaction between Rh^{F} and Rh^{E} forms Rp^{F}. Under favorable reaction conditions, a conjugation reaction between Rh^{J} and Rh^{E} forms Rp^{G}. Under favorable reaction conditions, a conjugation reaction between Rh^{N} and Rh^{A} forms Rp^{I} or Rp^{J} where U⁷ is O. Under favorable reaction conditions, a conjugation reaction between Rh^{N} and Rh^{B} forms Rp^{I} or Rp^{J} where U⁷ is S. Under favorable reaction conditions, a conjugation reaction between Rh^{N} and Rh^{C} forms Rp^{I} or Rp^{J} where U⁷ is N. Under favorable reaction conditions, a conjugation reaction between Rh^{O} and Rh^{A} forms Rp^{A} where U⁰ is O. Under favorable reaction conditions, a conjugation reaction between Rh^{O} and Rh^{B} forms Rp^{A} where U⁰ is NH. Under favorable reaction conditions, a conjugation reaction between Rh^{I} and Rh^{C} forms Rp^{K}. Under favorable reaction conditions, a conjugation reaction between Rh^{A} and Rh^{Q} forms Rp^{M} where U⁹ is O. Under favorable reaction conditions, a conjugation reaction between Rh^{A} and Rh^{R} forms Rp^{L} where U⁹ is O.

Conjugation reactions between cooperative handles may be done under favorable reaction conditions. Favorable reaction conditions are conditions that facilitate a reaction, increase the yield of a reaction, minimize unwanted biproducts of a reaction, and/or increase the rate of a reaction. Example reaction conditions include reaction temperature, reaction atmosphere composition, reaction solvent, the presence of a catalyst, the presence of a base, the presence of an acid, and any combination thereof. Favorable reaction conditions for conjugation reactions are known.

Cooperative handles may be chosen such that the conjugation reaction is an orthogonal conjugation reaction. Orthogonal conjugation reactions are reactions where the chemistry is selective such that only two cooperative handles react to form a reaction product even when additional reactive handles or pairs of cooperative reactive handles may be present. Orthogonal conjugation reactions may be useful because they allow for multiple selective conjugation reactions to take place in series or in parallel. Orthogonality of two or more conjugation reactions may be achieved by choosing reactive handles that are only reactive with their cooperative counterpart in the presence of other cooperative reactive handle pairs. Orthogonality of two or more conjugation reactions may also be achieved by using reactive handles that are reactive with multiple cooperative counterparts, but the reactivity can be influenced through the reaction conditions such that only a specific pair of cooperative handles will react in the given set of reaction conditions.

To form a separation ligand of formula SL1, conjugation reaction precursor compounds are employed, each precursor compound having a reactive handle that is cooperative with the reactive handle of a different precursor compound. In some embodiments, a separation ligand of formula SL1 is formed through the conjugation of a separation group precursor of formula Pre-Z(1) and a support substrate precursor of formula Pre-M(1) by way of synthetic scheme S1. The support substrate precursor includes a reactive handle Rh¹ that is covalently attached to the support substrate (shown as a thick black vertical line). The separation group precursor includes the separation group (Z) of formula SL1 and a separation group reactive handle Rh². Rh¹ and Rh² are cooperative reactive handles and may be any pair of cooperative handles as disclosed herein. In scheme S1, the support substrate reactive handle (Rh¹) is reacted with the separation group reactive handle (Rh²) to from a reaction product (Rp¹) thereby forming a separation ligand of formula SL1.

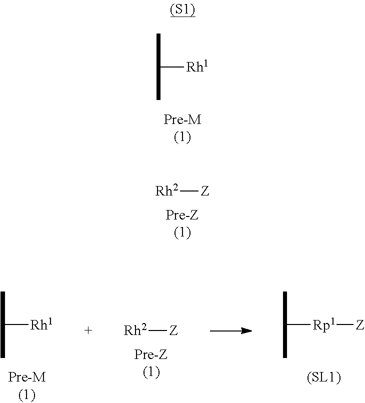

In some embodiments the material of the support substrate does not include a reactive handle that is cooperative with the separation group reactive handle (Rh²). In such embodiments, scheme S1 may further include installing the support substrate reactive handle Rh¹. The support substrate reactive handle Rh¹ may be installed through treatment of the support substrate to form the Rh¹. In such embodiments, a chemical functionality already present on the support substrate is transformed into the support substrate reactive handle. For example, the support substrate may be exposed to an oxidizing or reducing reagent (or conditions). The support substrate reactive handle Rh¹ may be installed through the installation of a functionalized layer. In such embodiments, the functionalized layer is considered a part of the support substrate. In such embodiments, the reactive handle of functionalized layer is the support substrate reactive handle. Examples of materials suitable for a functionalized layer are discussed herein.

In embodiments, where the separation group includes a carbohydrate binding domain or a protein that includes a carbohydrate binding domain, the separation group reactive handle may be the side chain of an amino acid. For example, in some embodiments, the separation group reactive handle is the amine of the side chain of lysine. In some embodiments, the separation group reactive handle is the hydroxyl side chain of the amino acid serine or threonine. In some embodiments, the separation group reactive handle is the thiol of the amino acid side chain of cysteine. Because proteins may have multiple amino acids of the same type, it may be difficult to control the location of the reactive handle on the affinity group. For this reason, in some embodiments, the plurality of separation groups having an affinity group may have some affinity groups attached to the support substrate at one reactive handle location and other affinity groups attached to the support substrate at a different reactive handle location. Additionally, in some embodiments, the plurality of separation groups having an affinity group may have some affinity groups attached to the support substrate with a first reaction product and others attached with a second reaction product.

In some embodiments, the carbohydrate binding domain or protein containing the same may be engineered to include an amino acid residue that has a reactive handle at a specific location on the protein (e.g., near the C or N terminus). In some such embodiments, the amino acid residue is a natural amino acid that has a side chain with a reactive handle (e.g., lysine, serine, threonine, cysteine). In other embodiments, the amino acid residue is an unnatural amino acid that has a side chain that includes a reactive handle. Examples of unnatural amino acids that have side chains with reactive handles include those that include an azide (e.g., 3-azido-alanine, 6-azido lysine, 4 azido phenylamine, (2S,4S)-Fmoc-4-azido-pyrrolidine-2-carboxylic acid, 2-(R)-Fmoc-amino-3-azidopropionic acid, and 4-(4-Azidophenyl)butyric acid) and those that include and alkyne (e.g., L-Homopropargylglycine). In some embodiments, where affinity group is engineered to include a reactive handle, the reactive handle may be separated from the affinity group by a linker. The linker may be an amino acid sequence.

In some embodiments, the linker (L) is of formula L2 such that SL is of formula SL2; that is:

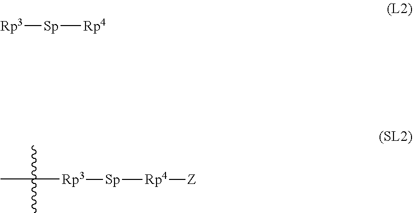

In formulas L2 and SL2, Sp is a spacer, Rp³ is a first reaction product, and Rp⁴ is a second reaction product. In formula SL2, Z is the separation group. The linker of formula L2 includes Sp, Rp³ and Rp⁴. In a separation ligand of formula SL2, a covalent bond from Rp³ to the support substrate is the point of covalent attachment of the linker (L2) to the support substrate. A covalent bond from Rp⁴ to the separation group (Z) is the point of covalent attachment of the linker (L2) to the separation group (Z). Rp³ and Rp⁴ may be any reaction product as described herein.

In some embodiments where the separation ligand is of formula SL2, Rp³, Rp⁴, or both are Rp^{A} where U⁰ is NH. In some such embodiments, the amide nitrogen (U⁰) of Rp^{A} is covalently linked to the support substrate or covalently linked to the separation group. In other such embodiments, the amide nitrogen of Rp^{A} is covalently linked to the spacer.

In some embodiments where the separation ligand is of formula SL2, Rp⁴ is Rp^{L} where U⁸ is O. In some such embodiments, oxygen is covalently linked to an affinity group. In some such embodiments, the affinity group comprises thiosalicylic acid; 4-mercaptophenyl boronic acid; 4-mercaptophenol; gallic acid; serotonin; epigallocatechin gallate (EGCG; 6); reactive red 120, cibracon brilliant yellow 3G-P; or reactive blue 4.

In some embodiments where the separation ligand is of formula SL2, Rp⁴ is Rp^{M} where U⁹ is O and R is a halogen. In some such embodiments, the oxygen is covalently linked to the separation group. In other such embodiments, the oxygen is covalently linked to the support substrate. In some such embodiments, the affinity group comprises thiosalicylic acid; 4-mercaptophenyl boronic acid; 4-mercaptophenol; gallic acid; serotonin; epigallocatechin gallate (EGCG; 6); reactive red 120, cibracon brilliant yellow 3G-P; or reactive blue 4.

In some embodiments where the separation ligand is of formula SL2, Rp³, Rp⁴, or both are Rp^{A} where U⁰ is O. In some such embodiments, the ester oxygen (U⁰) of Rp^{A} is covalently linked to the support substrate or covalently linked to the separation group. In other such embodiments, the ester oxygen of Rp^{A} is covalently linked to the spacer.

The spacer (Sp) of the linker (L1) may be of any length and/or chemical composition that does not completely inhibit the formation of the first reaction product and the second reaction product. The spacer (Sp) may be of any length and/or chemical composition that does not completely inhibit the ability of the affinity group to bind to its intended target.

The spacer (Sp) includes a divalent organic group. The divalent organic group includes a backbone. The “backbone” is the longest contiguous chain of atoms within the spacer (Sp). In some embodiments, the backbone is a carbon-based backbone. A backbone that is carbon-based is a backbone that has a greater number of carbon atoms than heteroatoms in the backbone. The backbone may include one or more substitutions extending from the backbone and/or one or more functional groups catenated within the backbone.

In some embodiments, the backbone is an alkanediyl (divalent group that is a radical of an alkane) or an alkenediyl (divalent group that is a radical of an alkene). The alkanediyl or alkenediyl may have a backbone chain length of C1 to C18, C1 to C10, C1 to C6, C1 to C4, C1 to C3, or C2 to C4. An alkenediyl may have one or more double bonds. The one or more double bonds may be located at any point along the backbone.

In some embodiments, the backbone includes one or more catenated functional groups. Catenated functional groups have at least one atom that is a part of the backbone; that is, at least one atom of the functional group lies within the backbone chain. The at least one atom of the functional group that is a part of the backbone can be a carbon or a heteroatom. For example, in some embodiments, the backbone includes a catenated ketone where the carbon atom of the carbonyl of the ketone is a part of the backbone. In other embodiments, the backbone includes a catenated amide. In some such embodiments, the nitrogen of the catenated amide is a part of the backbone and the carbon of the carbonyl is not a part of the backbone. In other such embodiments, the nitrogen and the carbonyl carbon of the amide are a part of the backbone. Example catenated functional groups include, ethers; thioether; esters (where the ester oxygen atom is a part of the backbone, or where the ester oxygen and the carbonyl carbon are a part of the backbone); thioesters (where the thioester sulfur atom is a part of the backbone, or where the thioester sulfur atom and the carbonyl carbon are a part of the backbone); amides (where the amide nitrogen is a part of the backbone, or where the amide nitrogen and the carbonyl carbon are a part of the backbone); ureas (where one of the urea nitrogens is a part of the backbone, or where both of the urea nitrogens and the carbonyl carbon are a part of the backbone); carbamates (where the carbamate oxygen is a part of the backbone; the carbamate nitrogen is a part of the backbone; or the carbamate oxygen, the carbamate nitrogen, and the carbonyl carbon are a part of the backbone); thioureas (where one of the urea nitrogens is a part of the backbone, or where both of the urea nitrogens and the carbonyl carbon are a part of the backbone); secondary and tertiary amines; aromatic rings (where at least two atoms of the aromatic ring are a part of the backbone); and any combination thereof.

In some embodiments the spacer a catenated ethers (i.e., a catenated oxygen atoms). In some such embodiments, the backbone includes a polyethylene glycol chain of 1, 2, 3, 4, 5, 6, 7, 8, 9, or 10 —OCH₂CH₂— repeat units. In some embodiments, the spacer includes a catenated ketone.

In some such embodiments, the spacer is of the formula —(CO)— (i.e., the backbone is a C1 alkenediyl and the C1 is the carbonyl carbon of the catenated ketone).

In some embodiments where the separation ligand is of formula SL2, Rp³ and Rp⁴ are both Rp^{E} where each U⁵ is independently O, NH, or S. In some embodiments, the U⁵ of Rp³ is O and the U⁵ of Rp⁴ is O. In some embodiments, the U⁵ of Rp³ is NH and the U⁵ of Rp⁴ is NH. In some embodiments, the U⁵ of Rp³ is O and the U⁵ of Rp⁴ is NH. In some embodiments, the U⁵ of Rp³ is NH and the U⁵ of Rp⁴ are O.

In some embodiments where Rp³ and Rp⁴ are both Rp^{E}, Sp may be —C(O)—. In some such embodiments, L2 may be described as Rp^{B}. In some embodiments were L2 is Rp^{B}, U² is O. In some embodiments were L2 is Rp^{B}, U¹ is O. In some embodiments were L2 is Rp^{B}, U³ is O. In some embodiments were L2 is Rp^{B}, U¹ is NH. In some embodiments were L2 is Rp^{B}, U³ is NH. In some embodiments were L2 is Rp^{B}, U¹ is O, U² is O, and U³ is NH. In some embodiments were L2 is Rp^{B}, U¹ is NH, U² is O, and U³ is O.

In some embodiments where the separation ligand is of formula SL2, Rp³ is Rp^{E} and Rp⁴ is Rp^{I} or Rp^{J} where U⁵ and U⁷ are each independently O, NH, or S. In some embodiments U⁵ is NH and U⁷ is NH. In some embodiments U⁵ is O and U⁷ is O. In some embodiments U⁵ is NH and U⁷ is O. In some embodiments U⁵ is O and U⁷ is NH.

In some embodiments where Rp³ and Rp⁴ are both Rp^{E}, Sp may be —(CH₂)ₙ— where n is 0, 1, 2, 3, 4, 5, 6, 7, 8, 9, or 10. In some such embodiments, L2 is of the formula or

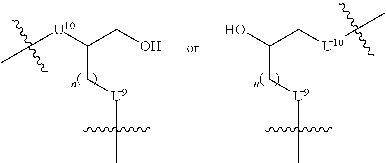

where U⁹ and U¹⁰ are each independently O, NH, or S and n is 0, 1, 2, 3, 4, 5, 6, 7, 8, 9, or 10. U⁹ may be U⁵ from Rp^{I} and U¹⁰ may be U⁷ from Rp^{I}. In some embodiments U⁹ is NH and U¹⁰ is NH. In some embodiments U⁹ is O and U¹⁰ is O. In some embodiments U⁹ is NH and U¹⁰ is O. In some embodiments U⁹ is O and U¹⁰ is NH.

In some embodiments, a separation ligand of formula SL is of formula

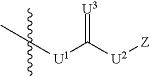

where U¹, U², and U³ are each independently O, NH, or S and Z is a separation group. For example, in some embodiments, a separation ligand of formula SL is

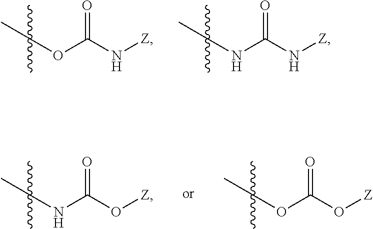

where Z is a separation group. In some embodiments, Z is an affinity group.

In some embodiments, a separation ligand of formula SL is of formula

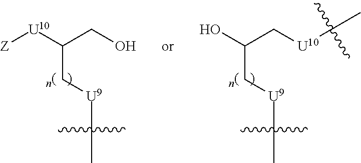

where U⁹ and U¹⁰ are each independently O, NH, or S where Z is a separation group. In some embodiments, SL is

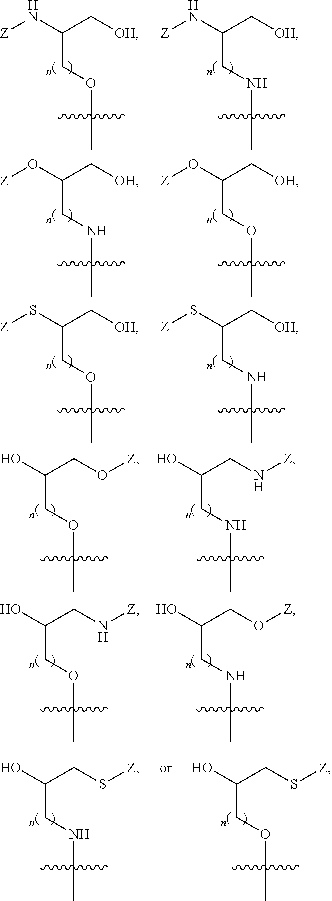

where Z is a separation group and n is 0, 1, 2, 3, 4, 5, 6, 7, 8, 9, or 10. In some embodiments Z is an affinity group.

To form a separation ligand of formula SL2, a series of conjugation reaction precursor compounds are employed, each precursor compound having a reactive handle that is cooperative with the reactive handle of a different precursor compound. In some embodiments, a separation media of formula SL2 is formed through the conjugation of a linker precursor of formula Pre-L, an affinity group precursor of formula Pre-Z(2), and a support substrate precursor of formula Pre-M(2). The linker precursor (Pre-L) includes the spacer Sp of the separation media of formula SL2, a first linker reactive handle Rh³, and a second linker reactive handle Rh⁴. The support substrate precursor (Pre-M(2)) includes a support substrate (vertical black line) and a support substrate reactive handle Rh⁵. The separation group precursor (Pre-Z(2)) includes the separation group Z of formula SL2 and a separation group reactive handle Rh⁶.

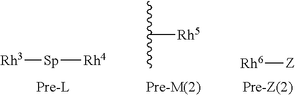

Rh³ and Rh⁵ are a pair of cooperative reactive handles. Rh⁴ and Rh⁶ are a pair of cooperative reactive handles. Rh³ of the linker precursor reacts with Rh⁵ of the support substrate precursor in a conjugation reaction to from a reaction product (i.e., Rp³ of formula SL2). Rh⁴ of the linker precursor reacts with Rh⁶ of the separation group precursor in a conjugation reaction to from a reaction product (i.e., Rp⁴ of formula SL2).

Because the linker precursor includes two reactive handles, the linker precursor is a bifunctional linker. In some embodiments, the linker precursor may be a multifunctional linker precursor that has three or more reactive handles. At least one of the reactive handles is configured to react with the support substrate precursor. The additional reactive handles may be configured to react with a cooperative reactive handle on one or more separation group precursor. Examples, of bifunctional and multifunctional linker precursors include, epichlorohydrin, diglycidyl ether, triglycidyl ether, tetraglycidyl ether, triazine, poly triazine, poly acrylic (e.g., the COOH groups can be made into activated ester reactive handles), succinic acid (e.g., the COOH groups can be made into activated ester reactive handles), and N′N′-disuccinimidyl carbonate (DSC).

In some embodiments, a separation ligand of formula SL2 may be formed through two conjugation reactions. The reactions may be conducted in any order or simultaneously. For example, in some embodiments, a separation ligand of formula SL2 is formed by way of synthetic scheme 2 (S2).

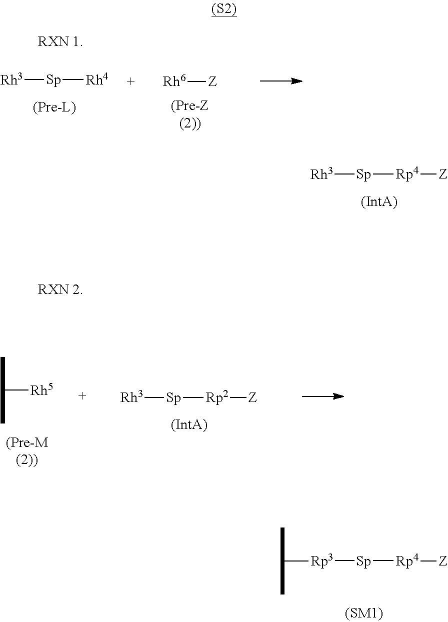

In a first conjugation reaction of scheme S2 (RXN1), the separation group reactive handle (Rh⁶) is reacted with a first linker reactive handle (Rh⁴) in a first conjugation reaction to from a first reaction product Rp⁴ thereby resulting in intermediate A (IntA). Intermediate A is a linker-separation group conjugate that includes the first reaction product (Rp⁴) and the second linker reactive handle (Rh³). IntA may be isolated or taken forward to the second conjugation reaction without isolation. In a second conjugation reaction of S2 (RXN 2) the second linker reactive handle (Rh³) of IntA is reacted with the support substrate reactive handle (Rh⁵) to form a second reaction product (Rp³), thereby forming a separation ligand of formula SL2.

In some embodiments, a separation ligand of formula SL2 is formed by way of synthetic scheme 3 (S3).

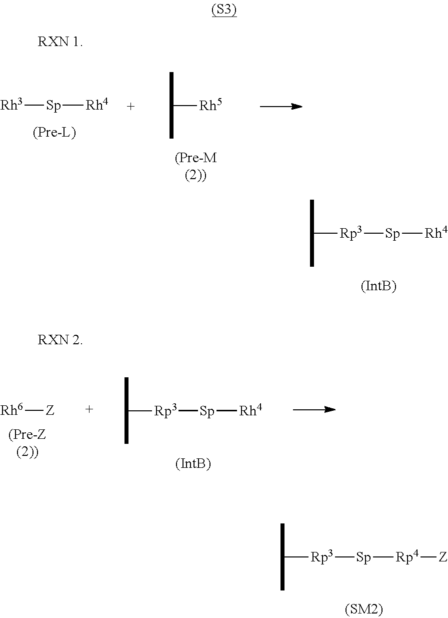

In a first conjugation reaction of scheme S3 (RXN1), the support substrate reactive handle (Rh⁵) is reacted with a first linker reactive handle (Rh³) in a first conjugation reaction to from a first reaction product (Rp³) thereby resulting in intermediate B (IntB). Intermediate B is a linker-support substrate conjugate that includes the first reaction product (Rp³) and the second linker reactive handle (Rh⁴). IntB may be isolated or taken forward to the second conjugation reaction without isolation. In a second conjugation reaction of S3 (RXN 2) the second linker reactive handle (Rh⁴) of IntB is reacted with the separation group reactive handle (Rh⁶) to form a second reaction product (Rp⁴), thereby forming a separation ligand of formula SL2.

Synthetic scheme S4 and synthetic scheme S5 are examples of forming a separation ligand of formula SL2 through scheme S3 using the bifunctional linker (Pre-L) N,N′-disuccinimidyl carbonate (S3) or epichlorohydrin (S4). In both S4 and S5, R¹⁰ can be OH, NH₂, or SH and R¹¹ can be O, NH, or S depending on the identity of R¹⁰.

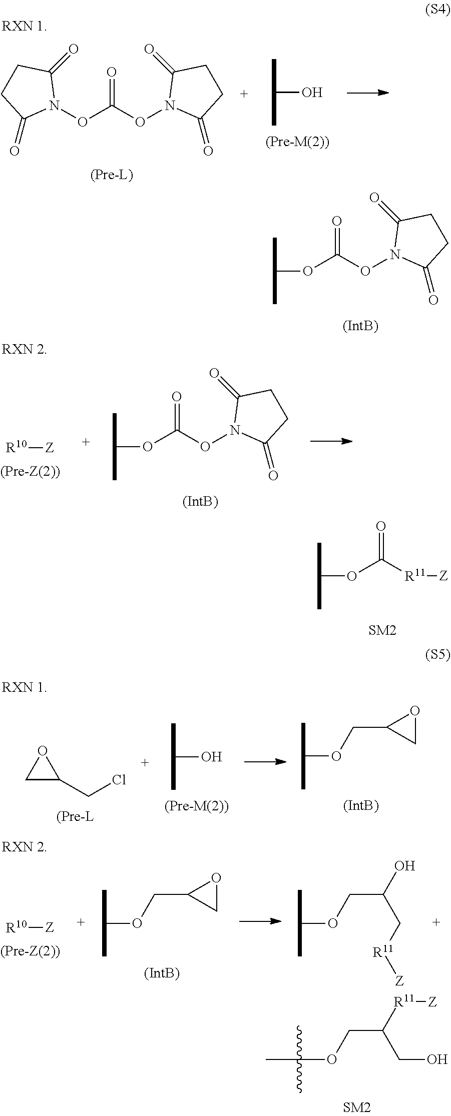

The present disclosure provides methods of making the separation media of the present disclosure. The separation media may be any separation media as disclosed herein. The separation media of the present disclosure may be made using methods similar to those described in PCT application number PCT/US2019/065805 (WO2020123714A1, Zhou), which is incorporated by reference in its entirety.

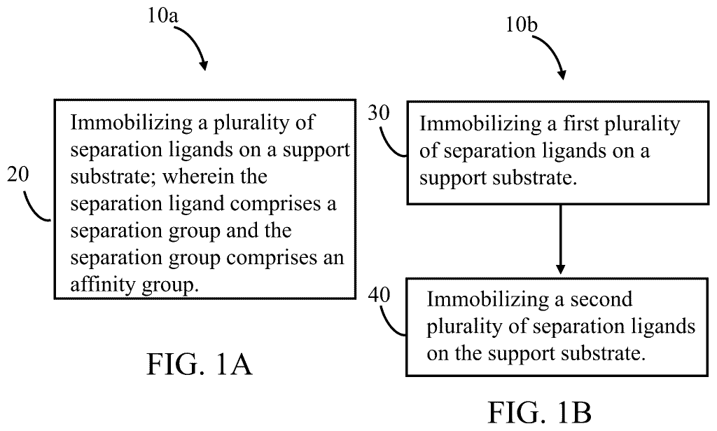

FIG. 1A is a flow diagram depicting a general method 10a for making a separation media of the present disclosure. The general method 10a includes immobilizing a plurality of separation ligands on a support substrate (step 20). Each separation ligand includes a separation group and a linker. The separation group includes the affinity ligand. Each separation ligand may be of formula SM, SM1, or SM2. Each separation ligand can be immobilized according to any relevant synthetic scheme described herein (e.g., S1, S2, S3, S4, or S5).

In some embodiments, the separation media includes a first plurality of separation ligands immobilized on the support substrate and a second plurality of separation ligands immobilized on the support substrate. FIG. 1B is a flow diagram depicting a general method 10b for making a separation media of the present disclosure that includes at least two pluralities of separation ligands. Each plurality of separation ligands immobilized on a support substrate may be of formula SLim. Each separation ligand of the first plurality of separation ligands and the second plurality of separation ligands includes a separation group and a linker. Each separation ligand of the first plurality of separation ligands and the second plurality of separation ligands may be of formula SM, SM1, or SM2. Each separation ligand of the first plurality of separation ligands and the second plurality of separation ligands can be immobilized according to any relevant synthetic scheme described herein (e.g., S1, S2, S3, S4, or S5).

The method 10b includes immobilizing the first plurality of separation ligands on a support substrate (step 30). The method 10b further includes immobilizing the second plurality of separation ligands on the support substrate (step 40).

In some embodiments of method 10b, the first plurality of separation ligands includes an assistance group, and the second plurality of separation ligands includes an affinity group. Without wishing to be bound by theory, it is thought that the assistance groups of the first plurality of separation ligands can interact with (e.g., via electrostatics and/or hydrophobic or hydrophilic interactions) with the affinity group of the separation group precursor used to form the second plurality of separation ligands. Through these interactions, the second separation group precursors may concentrate on the surface of the support substrate thereby increasing conjugation reaction efficiency (e.g., speed and/or yield). An increase in reaction efficiency may allow a lower concentration of the second plurality of the second separation group precursors to be used in the reaction step than would be needed to achieve the same reaction yield and/or surface coverage without the use of assistance groups. In some embodiments, the assistance group includes an amine. In such embodiments where separation ligands that include an amine assistance group are immobilized prior to immobilization of separation ligands containing affinity groups, the method is amine assisted.

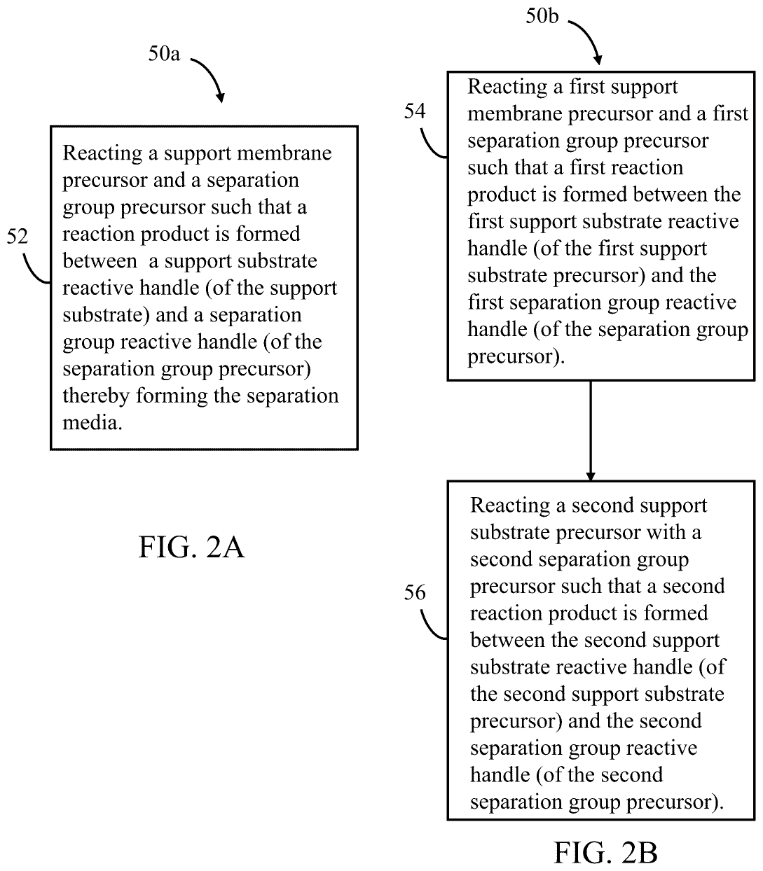

In some embodiments, method 10a or 10b may include method 50a. FIG. 2A is a flow diagram outlining method 50a for making a separation media including a separation ligand of the present disclosure. Method 50a may be understood in reference to synthetic scheme S1 as described herein; however, it is understood that method 50a is not limited to the synthetic scheme S1. The separation ligand of the separation media made from method 50a is synthesized from two components, a separation group precursor (e.g., Pre-Z(1)) and a support substrate precursor (e.g., Pre-M(1)). The separation group precursor includes the separation group (Z) and a separation group reactive handle (Rh²). The support substrate precursor includes a support substrate (thick vertical black line) and a support substrate reactive handle (Rh¹). The separation group reactive handle and the support substrate reactive handle are cooperative handles. Method 50a includes reacting a support substrate precursor and a separation group precursor such that a reaction product (e.g., Rp¹) is formed between the support substrate reactive handle (of the support substrate precursor) and the separation group reactive handle (of the separation group precursor) thereby forming the separation media (e.g., the immobilized separation ligand of Formula SLim)

In some embodiments, step 52 may be accomplished using a reaction mixture. The reaction mixture includes a solvent and the separation group precursor. The reaction mixture may be applied to the support substrate, or the support substrate may be submerged in the reaction mixture. The solvent may include an organic solvent, water, or both. In some embodiments, the solvent is an aqueous buffer that includes one or more salts and/or buffering agents as disclosed herein. The reaction mixture may include additional compounds that facilitate the reaction. For example, the reaction mixture may include an acid, a base, an initiator, a catalyst, or any combination thereof.

In some embodiments where the solvent includes an organic solvent, the reaction step is considered to be “organic assisted” or “organic solvent assisted.” In an organic assisted method, the solvent of the reaction mixture includes water and at least one water-miscible organic solvent. Examples of water-miscible organic solvents include ethanol, acetone, acetonitrile, methanol, propanol (e.g., 2-propanol, 1-propanol), 2-butanol, tetrahydrofuran, dimethylformamide, and dimethyl sulfoxide. The ratio of water to organic solvent in the reaction mixture is such that the reaction mixture is at or near the cloud point of the mixture. The cloud point is the point at which a liquid solution undergoes a liquid-liquid phase separation to from an emulsion or a liquid-solid phase transition to form a stable suspension or a precipitate. The cloud point can be visualized by observing the water-to-organic solvent ratio at which the reaction mixture becomes turbid. Without wishing to be bound by theory, it is thought that including an organic solvent in the reaction mixture such that the reaction mixture is at or near the cloud point increases the conjugation reaction efficiency. The organic solvent molecule can displace water molecules in the separation group precursor thereby increasing interactions between the separation group precursor and the support substrate.

It is possible to define a range of appropriate amounts of organic solvent in the reaction mixture in which the upper boundary is expressed by [V %_{cp}+a(100%−V %_{cp})] and the lower boundary is expressed by [V %_{cp}−bV %_{cp}], where “V %_{cp}” is the percent by volume of the organic solvent in the reaction mixture at the cloud point, “a” is the upper deviation from the cloud point, and “b” is the lower deviation from the cloud point. For the purpose of an example, if the percent by volume of the organic solvent in the ligand solution at the cloud point (V %_{cp}) is 60%, and the upper and lower boundaries are defined by a=0.3 and b=0.5, then the corresponding appropriate amounts of organic solvent in the reaction mixture would range from 30% to 72% organic solvent by volume. In embodiments, the reaction mixture can include an amount of organic solvent in which “a” is about 0.01, 0.02, 0.03, 0.04, 0.05, 0.06, 0.07, 0.08, 0.09, 0.1, 0.12, 0.14, 0.16, 0.18, 0.2, 0.25, 0.3, 0.4, 0.5, 0.6, 0.7, 0.8, 0.9, 0.99 and “b” is about 0.01, 0.02, 0.03, 0.04, 0.05, 0.06, 0.07, 0.08, 0.09, 0.1, 0.12, 0.14, 0.16, 0.18, 0.2, 0.25, 0.3, 0.4, 0.5, 0.6, 0.7, 0.8, 0.9, 0.99. In some embodiments, the reaction mixture includes an amount of organic solvent ranging from 70% to 130%, 80% to 120%, 90% to 110%, or 95% to 105% of the volumetric amount of the organic solvent at the cloud point of the reaction mixture.

In some embodiments where the reaction mixture is aqueous and includes one or more salts, the reaction step may be kosmotropic salt assisted. In a kosmotropic salt assisted method, the reaction mixture includes water and at least one kosmotropic salt at a concentration such that the reaction mixture is at or near its cloud point. Examples of kosmotropic salts include sodium phosphate, sodium sulfate, and ammonium sulfate. Without wishing to be bound by theory, it is thought that including a kosmotropic salt in the reaction mixture such that the reaction mixture is at or near the cloud point increases the conjugation reaction efficiency. The salt molecules can disrupt the solvation shell of separation group precursors thereby increasing interactions between the separation group precursor and the support substrate.

In some embodiments, the separation media includes a first plurality of separation ligands immobilized on the support substrate and a second plurality of separation ligands immobilized on the support substrate. In some such embodiments, method 50b may be used to prepare the separation media. FIG. 2B is a flow diagram outlining method 50b for making a separation media that includes multiple pluralities of separation ligands immobilized to the support substrate. The first plurality of separation ligands synthesized according to method 50b are made from two components a first support substrate reactive precursor and a first separation group precursor. The second plurality of separation ligands synthesized according to method 50b are made from two components a second support substrate reactive precursor and a second separation group precursor. The first support substrate precursor includes the first support substrate reactive handle. The second support substrate precursor includes a second support substrate reactive handle. The first support substrate reactive handle and the second support substrate reactive handle may be the same or different. The first separation group precursor includes a separation group and a first separation group reactive handle. The second separation group precursor includes a separation group and the second separation group reactive handle.

The first support substrate reactive handle and the first separation group reactive handle are cooperative reactive handles. The second support substrate reactive handle and second separation group reactive handle are cooperative handles.

Method 50b includes reacting the first support substrate precursor and the first separation group precursor such that a first reaction product is formed between the first support substrate reactive handle (of the first support substrate precursor) and the first separation group reactive handle (of the first separation group precursor). The method further includes reacting the second support substrate precursor with the second separation group precursor such that a second reaction product is formed between the second support substrate reactive handle (of the second support substrate precursor) and the second separation group reactive handle (of the second separation group precursor).

In some embodiments, the first separation group precursor includes an amine, and the entire method (50b) is amine assisted.

In some embodiments, step 54, step 56, or both are organic solvent assisted or kosmotropic salt assisted. For example, step 54 may be accomplished with a first reaction mixture that includes the first separation group precursor, water, and an organic solvent that is miscible with water or a kosmotropic salt. Step 56 may be accomplished with a second reaction mixture. The second reaction mixture includes the second separation group precursor water and an organic solvent that is miscible with water or at least one kosmotropic salt.

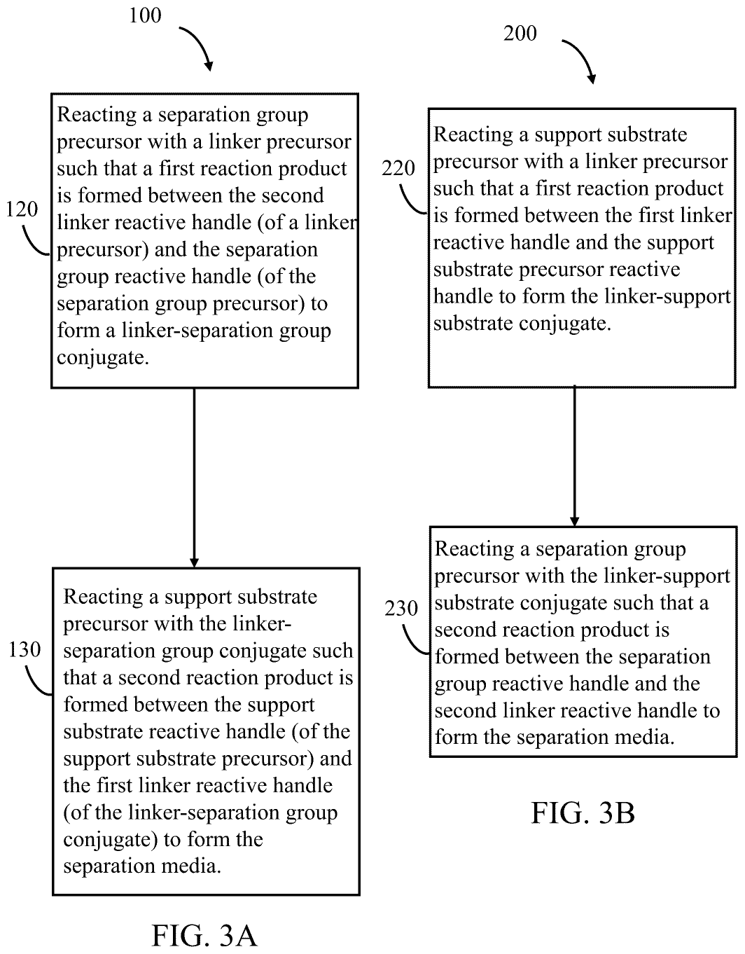

In some embodiments, method 10a,10b, 50a, or 50b may include method 100. FIG. 3A is a flow diagram outlining method 100. Method 100 may be understood in reference to synthetic scheme S2 as described herein; however, it is understood that the method of 100 is not limited to the synthetic scheme S2. The separation ligands of the separation media made according to method 100 are synthesized from three components, a linker precursor (e.g., Pre-L), a support substrate precursor (e.g., Pre-M(2)), and a separation group precursor (e.g., Pre-Z(2)). The linker precursor includes a first linker reactive handle (Rh³), a second linker reactive handle (Rh⁴), and a spacer (Sp) that covalently links the first linker reactive handle and the second linker reactive handle. The separation group precursor includes a separation group (Z) and a separation group reactive handle (Rh⁶). The support substrate precursor includes a support substrate (thick vertical black line) and a support substrate reactive handle (Rh⁵). The second linker reactive handle (Rh⁴) and the separation group reactive handle (Rh⁶) are cooperative reactive handles. The first linker reactive handle (Rh³) and the support substrate reactive handle (Rh⁵) are cooperative reactive handles.

The method 100 includes reacting the separation group precursor with the linker precursor such that a first reaction product is formed between the second linker reactive handle (of a linker precursor) and the separation group reactive handle (of the separation group precursor) to form a linker-separation group conjugate (step 120). Method 100 further includes reacting the support substrate precursor with the linker-separation group conjugate of step 120 such that a second reaction product is formed between the support substrate reactive handle (of the support substrate precursor) and the first linker reactive handle (of the linker-separation group conjugate) to form the separation media (step 130).

In some embodiments, method 10a, 10b, 50a, or 50b may include the method 200. FIG. 3B is a flow diagram outline method 200. The method 200 may be understood in reference to synthetic scheme S3 as described herein; however, it is understood that the method of 200 is not limited to the synthetic scheme S3. The separation ligand made according to method 200 is synthesized from three components, a linker precursor (e.g., Pre-L), a support substrate precursor (e.g., Pre-M(2)), and a separation group precursor (e.g., Pre-Z(2)). The linker precursor includes a first linker reactive handle (Rh³), a second linker reactive handle (Rh⁴), and a spacer (Sp) that covalently links the first linker reactive handle and the second linker reactive handle. The separation group precursor includes a separation group (Z) and a separation group reactive handle (Rh⁶). The support substrate precursor includes a support substrate (thick vertical black line) and a support substrate reactive handle (Rh⁵). The second linker reactive handle (Rh⁴) and the separation group reactive handle (Rh⁶) are cooperative reactive handles. The first linker reactive handle (Rh³) and the support substrate reactive handle (Rh⁵) are cooperative reactive handles.

Method 200 includes reacting a support substrate precursor with a linker precursor such that a first reaction product is formed between the first linker reactive handle and the support substrate precursor reactive handle to form the linker-support substrate conjugate. Method 200 further includes reacting a separation group precursor with the linker-support substrate conjugate such that a second reaction product is formed between the separation group reactive handle and the second linker reactive handle to form the separation media.

Any step of method 100 or method 200 may be organic solvent assisted or kosmotropic salt assisted.

In some embodiments, methods 10a, 10b, 50a, 50b, 100, and 200 further include functionalizing the support substrate to install the support substrate reactive handles. Installing the support substrate reactive handles followed by one or more conjugation reactions to immobilize the separation ligands to the reactive handles is called indirect immobilization. In such embodiments, the method may further include depositing a polymer having reactive handles onto the support substrate. In some embodiments, the polymer is deposited such that is grafted onto the support substrate. In other embodiments, the polymer is deposited such that it is grafted from the support substrate. In embodiments where the polymer is grafted from the support substrate, the method may further include coupling an initiator to the support substrate to form an immobilized initiator. In such embodiments, the method may further include polymerizing a plurality of monomers from the immobilized initiator.

In some embodiments, the support substrate reactive handle is already a part of the support substrate and not from a deposited functional layer. In such embodiments, the separation ligands are immobilized directly to the support substrate in a process called direct immobilization. Any of the methods 10a, 10b, 50a, 50b, 100, and 200 may include direct immobilization.

Direct and indirect immobilization may be accomplished using the amine assisted method, without amine assistance groups (not amine assisted), using the organic solvent assistance method, not using the organic solvent assistance method, using the kosmotropic assisted method, not using the kosmotropic salt assisted method, or any combination thereof.

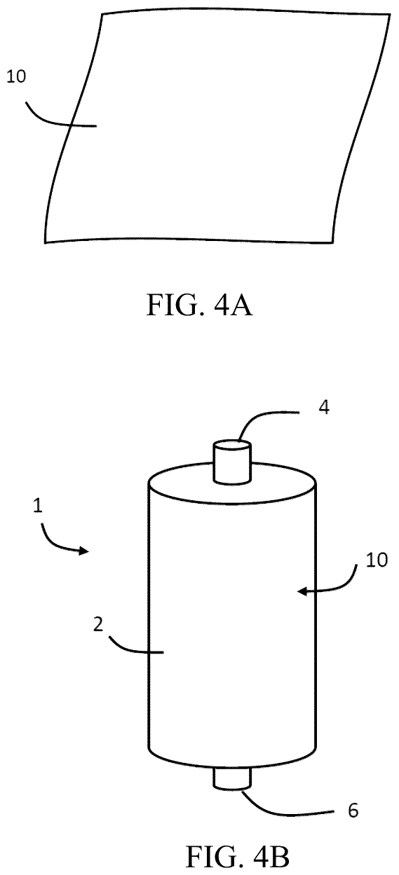

The separation media of the present disclosure may be employed in a separation device. The separation device is a membrane chromatography column, a membrane chromatography cassette, or other membrane chromatography device that includes the separation media of the present disclosure. A separation device may be operated manually or integrated with software, pumps, detectors, and other accessories. The separation media 10 is schematically shown as a membrane in FIG. 4A. The separation media membrane 10 may be provided in a separation device 1 (e.g., a chromatography column), shown in FIG. 4B.

In some embodiments, two or more separation media of the present disclosure may be arranged in a stacked configuration. The stacked configuration may be employed in a separation device. In some embodiments, a first separation media and a second separation media are arranged in a stacked configuration. In some embodiments, the first separation media and the second separation media have the same identity; that is, the separation media have the same support substrate and the same separation ligands immobilized on the substrate. The separation ligands are immobilized at the same or similar separation ligand densities. In other embodiments, the first separation media and the second separation media have different identities. For example, the first separation media and the second separation media have a different support substrate; different separation ligands; different separation group densities; or any combination thereof.

The separation device (e.g., membrane chromatography column, membrane chromatography cassette, or other membrane chromatography device) may provide a residence time of 5 minutes or less, 2 minutes or less, 1 minute or less, 30 seconds or less, 10 seconds or less, 6 seconds or less, 5 seconds or less, 4 seconds or less, 3 seconds or less, 2 seconds or less, or 1 second or less. The separation device (e.g., membrane chromatography column, membrane chromatography cassette, or other membrane chromatography device) may provide a residence time of 0.01 seconds or greater, 0.1 seconds or greater, 1 second or greater, 5 seconds or greater, 6 seconds or greater, 10 seconds or greater, 30 seconds or greater, 1 minute or greater, or 2 minutes or greater. Residence time is the time any normalized amount of fluid takes to traverse the separation media of the separation device (a single separation media or multiple separation media). For example, residence time is the time it takes any molecule that is not the target and/or does not bind to the separation media to traverse the separation media in a separation device. Residence time is calculated as the flow rate or the solution going through the column divided by the total bed volume of all of the separation media included in the separation device. The residence times of the separation devices of the present disclosure may be lower than those of separation media made of resins. According to an embodiment, using membrane-based purification devices can significantly improve productivity.

Process productivity can be defined using the equation below. In the denominator, Vₜₒₜ is the total volume of solution passing through the separation media (e.g., column or cassette) during the whole process, including load (the volume of the isolation solution discussed herein), rinse (e.g., the volume of the washing solution as discussed herein), elution (e.g., the volume of the elution solution as discussed herein), and regeneration steps (e.g., the volume of the regeneration solution as discussed herein). BV is the chromatography medium bed volume (corresponding to the volume of the separation media), and τ (tau) is residence time. Loading volume is proportional to dynamic binding capacity of the chromatography column medium. Thus, process productivity increases with increasing binding capacity and decreasing residence time.

$$Productivity = \frac{target}{Cost of time} = \frac{Loading volume \times target \times yield}{( \frac{{V}_{tot}}{BV} ) \times τ}$$

Dynamic binding capacity generally refers to the concentration of bound target on the separation media (milligram bound per unit bed volume of separation media) at breakthrough in the effluent. A dynamic binding capacity at 10% breakthrough (DBC_{10%}) can be determined via a standard chromatography method, e.g., using Cytiva ÄKTA pure Fast Protein Liquid chromatography (FPLC). First, the separation media is packed into a housing unit. Then, the contained separation media is connected to an FPLC system. Next, feed material (e.g., isolation solution) containing the target is passed though the separation media under certain column volumes per minute flowrate (CV/min) until the effluent concentration of the target reaches 10% of the feed concentration, as determined by a detector (e.g., a UV detector). At the end, based on the holdup volume in the FPLC system and separation media volume, the DBC_{10%} is calculated as follows ((Volume to 10% breakthrough-holdup volume)×(feed concentration))/(volume of separation media)=DBC_{10%} expressed as mg target material/unit volume separation media. The volume of the separation media is determined by the surface area of the separation media multiplied by the thickness of the separation media. The volume of the separation media can be referred to as the bed volume. In general, the volume of the separation media does not account for the void space within the separation media. The holdup volume is the total volume between the injection port (i.e., the location where a fluid enters the system) and the detector. The holdup volume includes the bed volume (e.g., the separation media volume) as well as any volume between the injection port and the bed and any volume between the bed and the detector.

In some embodiments, a separation media or separation device containing the same has a dynamic binding capacity at 10% breakthrough of 0.01 milligrams of target per 1 mL bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media has a dynamic binding capacity at 10% breakthrough of 150 mg/mL bed volume or less, 120 mg/mL bed volume or less 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a dynamic binding capacity at 10% breakthrough of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume. The dynamic binding capacity may depend at least in part on the target and the affinity group.

The separation media of the present disclosure may have a variety of static binding capacities (SBC). The static binding capacity is the amount of target bound to the separation media per volume of the separation media. The static binding capacity can be determined, for example, by incubating the separation media with an isolation solution containing a known amount of the target ligand for a period of time. Following incubation, the amount of the target still in the isolation solution (target not bound to the separation media) can be measured. The static binding capacity can then be calculated as the difference between the initial amount of the target in the isolation solution and the amount of target in the isolation solution following incubation with the separation media. The amount of the target in the isolation solution pre- and post-incubation with the separation media can be determined, for example, using spectroscopy and/or high performance liquid chromatography.

The static binding capacity may be higher than the dynamic binding capacity at 10% breakthrough. For example, in some embodiments, the SBC can be 10% to 40% greater than the DBC_{10%}. The pore size of the support substrate may influence the SBC and DBC_{10%}. For example, smaller pore sizes may cause a greater difference between the SBC and the DBC_{10%} as compared to relatively larger pore sizes. In some embodiments, a separation media or separation device containing the same has a static binding capacity of 0.01 milligrams of target per 1 mL of bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media or separation device containing the same has a static binding capacity of 150 mg/mL bed volume or less, 120 mg/mL bed volume or less 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a static binding capacity of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume. The static binding capacity may depend at least in part on the target and the affinity group.

The separation media may have a variety of separation ligand densities. Separation ligand density is the amount of separation ligands immobilized per unit volume of the separation media. In embodiments where the separation media only includes separation groups that include affinity groups, the separation group density can be a measure of affinity group density. The separation ligand density can be determined, for example, by incubating the support substrate (for example, according to S1, S2, or S3) with the reaction solution containing a known amount of the separation group precursor for immobilization for a reaction time to form the separation media. Following incubation, the amount of the separation group precursor containing still in the reaction solution (unreacted) can be measured. The density of the separation ligands can then be calculated as the difference between the initial amount of the separation group precursor in the reaction solution and the amount of separation group precursor in the reaction solution following incubation with the support substrate. The amount of the separation group precursor in the reaction solution pre- and post-incubation with the support substrate can be determined, for example, using spectroscopy and/or high performance liquid chromatography. The separation group pre-cursor can be used as a proxy for the separation ligand.

In some embodiments, the separation media has separation ligand density of 0.01 milligrams of separation ligand per 1 mL of bed volume (mg/mL bed volume) or greater, 0.1 mg/mL bed volume or greater, 1 mg/mL bed volume or greater, 5 mg/mL bed volume or greater, 10 mg/mL bed volume or greater, 20 mg/mL bed volume or greater, 30 mg/mL bed volume or greater, 40 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, 110 mg/mL bed volume or greater, or 120 mg/mL bed volume or greater. In some embodiments, a separation media has a separation ligand density of 150 mg/mL bed volume or less, 120 mg/mL bed volume or less, 110 mg/mL bed volume or less, 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 30 mg/mL bed volume or less, or 20 mg/mL bed volume or less, 10 mg/mL bed volume or less, 5 mg/mL bed volume or less, 1 mg/mL bed volume or less, or 0.1 mg/mL bed volume or less. In some embodiments, a separation media has a separation ligand density of 0.01 mg/mL bed volume to 150 mg/mL bed volume, 0.1 mg/mL bed volume to 150 mg/mL bed volume, 1 mg/mL bed volume to 150 mg/mL bed volume, 5 mg/mL bed volume to 150 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 10 mg/mL bed volume to 20 mg/mL bed volume, 15 mg/mL bed volume to 60 mg/mL bed volume, 15 mg/mL bed volume to 50 mg/mL bed volume, 15 mg/mL bed volume to 30 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 20 mg/mL bed volume to 30 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, or 1 mg/mL bed volume to 10 mg/mL bed volume.

Separation ligand density can also be described as the specific surface area (SSA) in square meters (m²) relative to the bed volume of the separation media. SSA can be determined, for example, using nitrogen Brunauer-Emmett-Teller (BET) analysis. Prior to immobilization of the separation ligands on the support substrate, the support substrate will have a support substrate SSA. After immobilization of the separation ligands on the support substrate to form the separation media, the separation media has a separation media SSA. The support substrate SSA and the separation media SSA may be impacted by the pore size of the support substrate, Generally, support substrates with greater pore sizes have a larger support substrate SSA. Generally, the separation media SSA will be greater than the support substrate SSA. In some embodiments the separation media SSA is 0.5 times or greater than the support substrate SSA, 1 time or greater than the support substrate SSA, 1.5 times or greater than the support substrate SSA, 2 times or greater than the support substrate SSA, 3 time or greater than the support substrate SSA, 4 time or greater than the support substrate SSA, 5 time or greater than the support substrate SSA, or 7 time or greater than the support substrate SSA. In some embodiments the separation media SSA is 10 times or less than the support substrate SSA, 7 times or less than the support substrate SSA, 5 times or less than the support substrate SSA, 4 times or less than the support substrate SSA, 3 times or less than the support substrate SSA, 2 times or less than the support substrate SSA, 1.5 times or less than the support substrate SSA, or 1 time or less than the support substrate SSA.

In some embodiments the separation media has a separation SSA of 1.5 meters squared per milliliter of bed volume (m²/mL bed volume) or greater, 2 m²/mL bed volume or greater, 3 m²/mL bed volume or greater, 4 m²/mL bed volume or greater, 5 m²/mL bed volume or greater, 8 m²/mL bed volume or greater, 9 m²/mL bed volume or greater, 10 m²/mL bed volume or greater, or 15 m²/mL bed volume when the support substrate has an average pore size of 0.1 μm to 10.0 μm, such as 0.2 μm to 0.5 μm. In some embodiments the separation media has a separation SSA of 20 m²/mL bed volume or less, 15 m²/mL bed volume or less, 10 m²/mL bed volume or less, 9 m²/mL bed volume or less, 8 m²/mL bed volume or less, 7 m²/mL bed volume or less, 6 m²/mL bed volume or less, 5 m²/mL bed volume or less, 4 m²/mL bed volume or less, or 3 m²/mL bed volume or less, 2 m²/mL bed volume or less when the support substrate has an average pore size of 0.1 μm to 10.0 μm, such as 0.2 μm to 0.5 μm. In some embodiments the separation media has a separation SSA of 1.5 m²/mL bed volume to 20 m²/mL bed volume, 1.5 m²/mL bed volume to 15 m²/mL, 1.5 m²/mL bed volume to 10 m²/mL, 2 m²/mL bed volume to 20 m²/mL, 2 m²/mL bed volume to 15 m²/mL, 2 m²/mL bed volume to 10 m²/mL, 2 m²/mL bed volume to 9 m²/mL, 2 m²/mL bed volume to 8 m²/mL, 2 m²/mL bed volume to 7 m²/mL, 2 m²/mL bed volume to 6 m²/mL, 2 m²/mL bed volume to 5 m²/mL, 3 m²/mL bed volume to 20 m²/mL, 3 m²/mL bed volume to 15 m²/mL, 3 m²/mL bed volume to 10 m²/mL, 4 m²/mL bed volume to 20 m²/mL, 4 m²/mL bed volume to 15 m²/mL, 4 m²/mL bed volume to 10 m²/mL, 5 m²/mL bed volume to 20 m²/mL, 5 m²/mL bed volume to 15 m²/mL, or 5 m²/mL bed volume to 10 m²/mL when the support substrate has an average pore size of 0.1 μm to 10.0 μm, such as 0.2 μm to 0.5 μm.

In some embodiments, the separation media and/or separation devices containing the same are able to purify a target at fast flow rates. For example, separation media and/or separation devices containing the same may be used to purify a target at residence times of 5 minutes of less, 2 minutes or less, 1 minute or less, 30 seconds or less, 10 seconds or less, or 6 seconds or less. The residence time is somewhat dependent on the volume of the separation media and/or on the size of the device. For example, in separation media that have low volumes and/or separation devices that are small, the residence times may be as low as 1 second or less. Although there is no desired lower limit for the residence time, in practice residence times are 0.1 seconds or greater.

In some embodiments, the separation media and/or separation device containing the same may be used to purify or concentrate a target from an isolation solution with a high recovery of the target molecule. The recovery of a target molecule is amount of the target molecule recovered after passing it through the separation media divided by the amount of target molecule in the isolation solution. In some embodiments, the target molecule recovery is 50% or greater, 60% or greater, 80% or greater, 90% or greater, 95% or greater, or 99% or greater. In some embodiments, the target molecule recovery is 100% or less, 95% or less, 90% or less, 80% or less, 70% or less, or 60% or less. In some embodiments, the target molecule recovery is 80% to 100%, 90% to 100%, or 95% to 100%.

In some embodiments, the separation media and/or separation device containing the same may be used to purify or concentrate target models from an isolation solution with a high recovery of active target molecule. An active target molecule is a molecule that possesses at least some of the function as the target molecule prior to exposure to the separation media of the present disclosure. For example, an active target molecule is a target molecule that has undergone purification using the separation media of the present disclosure and retains at least some binding affinity to a binding partner. The recovery of active target molecules is the amount of active target molecule recovered after passing them through the separation media divided by the amount of target molecules in the isolation solution. In some embodiments, the active target molecule recovery is 50% or greater, 60% or greater, 80% or greater, 90% or greater, 95% or greater, or 99% or greater. In some embodiments, the active target molecule recovery is 100% or less, 95% or less, 90% or less, 80% or less, 70% or less, or 60% or less. In some embodiments, the active target molecule recovery is 80% to 100%, 90% to 100%, or 95% to 1000%.

In some embodiments, the separation media and/or separation device containing the same may be used to remove impurities from an isolation solution. An impurity is any molecule that is not the target molecule, or a buffering agent, a salt, or an additive that has been added to the isolation solution. In some embodiments, the separation media is able to remove 50% or greater, 60% or greater, 70% or greater, 80% or greater, 90% or greater, or 99% or greater of the impurities initially present in the isolation solution. In some embodiments, the separation media is able to remove 100% or less, 99% or less, 90% or less, 80% or less, 70% or less, or 60% or less. In some embodiments, the separation media is able to remove 50% to 100%, 60% to 100%, 70% to 100%, 80% to 100% 90% to 100%, 90% to⁹⁹%, or 90% to 95% of the impurities initially present in the isolation solution.

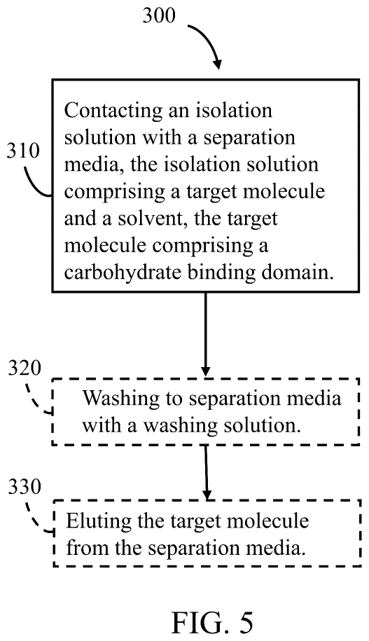

The present disclosure describes a method for using the separation media and/or the separation devices of the present disclosure. FIG. 5 is a flow diagram outlining a method 300 for using the separation media of the present disclosure to isolate and/or concentrate a target molecule from an isolation solution. Method 300 includes contacting an isolation solution with a separation media (step 310).

The isolation solution includes a solvent (isolation solvent) and a target molecule. In some embodiments, the isolation solution includes a plurality of target molecules that have already been purified from a mixture that included additional biomolecules. In such embodiments the plurality of target molecules may be already pure but not concentrated to the desired concentration in a given isolation solution. In some such embodiments, the separation media may be used to concentrate the plurality of target molecules by decreasing the volume of solution in which they are located. In such embodiments, the isolation solution may include one or more suitable buffering agents, one or more suitable salts, one or more suitable additives, or any combination thereof.

The target may be any target described herein. In some embodiments, the target includes a free protein. In some embodiments, the target includes an antibody or a fragment thereof. In some embodiments, the target includes fetuin, mucin, asialofetuin, or a fragment thereof.

In other embodiments, the separation media or separation device may be used to purify the target from a mixture that includes contaminant molecules or undesired molecules. In some such embodiments, the isolation solution includes a mixture of biomolecules and/or cellular debris. In addition, the isolation solution may include one or more suitable buffering agents, suitable salts, other suitable additives, or any combination thereof. For example, the isolation solution may include the lysate of an expression system used to produce the plurality of target molecules as well as any salts, buffering agents, or additives used to lyse the cells. The isolation solution may include the media (e.g., Dulbecco's modified eagle medium) of an expression system in which the target molecules have been excreted from.

Examples of suitable salts and buffering agents include sodium chloride; potassium chloride; lithium chloride; rubidium chloride; calcium chloride; magnesium chloride; cesium chloride; tris base (tris(hydroxymethyl)aminomethane); 4-(2-hydroxyethyl)-1-piperazineethanesulfonic acid (HEPES); 4-(2-hydroxyethyl)piperazine-1-propanesulfonic acid (EPPS); sodium phosphate; potassium phosphate; ammonium sulfate, 2-(N-morpholino)ethanesulfonic acid (MES); 2,2′,2″-nitrilotriacetic acid (ADA); N-(2-acetamido)-2-aminoethanesulfonic acid (ACES); 3-morpholino-2-hydroxypropanesulfonic acid (MOPSO); cholamine chloride hydrochloride; 3-(N-morpholino)propanesulfonic acid (MOPS); N,N-bis(2-hydroxyethyl)-2-aminoethanesulfonic acid (BES); 2-{[1,3-dihydroxy-2-(hydroxymethyl)propan-2-yl]amino}ethane-1-sulfonic acid (TES); 3-(N,N-bis [2-hydroxyethyl]amino)-2-hydroxypropanesulfonic acid (DIPSO); 3-[N-tris(hydroxymethyl)methylamino]-2-hydroxypropanesulfonic acid (TAPSO); acetamidoglycine; piperazine-1,4 BIS(2-hydroxypropanae sulphonic acid) (POPSO); N-(hydroxyethyl)piperazine-N′-2-hydroxypropanesulfonic acid (HEPPSO); 3-[4-(2-hydroxyethyl)piperazin-1-yl]propane-1-sulfonic acid (HEPPS); N-(tri(hydroxymethyl)methyl)glycine (tricine); 2-aminoacetamide; glycylglycine; N,N-bis(2-hydroxyethyl)glycine; N-tris(hydroxymethyl)methyl-3-aminopropanesulfonic acid (TAPS); and the like. Suitable salts and/or buffering agents may be added in an amount of 1 millimolar (mM) or greater, 5 mM or greater, or 10 mM or greater, 20 mM or greater, 50 mM or greater, 100 mM or greater 200 mM or greater, or 500 mM or greater. Suitable salts may be added in an amount of 1 M or less, 500 mM or less, 100 mM or less, 50 mM or less, or 30 mM or less. The salts may be added in an amount ranging from 1 mM to 1 M, 1 mM to 500 mM, 1 mM to 200 mM, 1 mM to 100 mM, 1 mM to 50 mM, 5 mM to 30 mM, 5 mM to 20 mM, or 20 mM to 100 mM.

In some embodiments, the isolation solution includes one or more kosmotropic salts, one or more chaotropic salts, or both. Kosmotropic salts are known as salts that decrease the solubility of nonpolar substances in aqueous solutions. In contrast, chaotropic salts increase the solubility of nonpolar substances in aqueous solutions. In some embodiments, the amount and/or identity of a kosmotropic and/or chaotropic salts may be designed to increase the binding affinity and/or binding specificity between the target molecule and the affinity groups and/or assistance groups (if present).

Examples of kosmotropic salts that may be present in the isolation solution include ammonium sulfate, ammonium phosphate, potassium phosphate, sodium sulfate, sodium chloride, and any combination thereof. Suitable kosmotropic salts may be present in the isolation solution in an amount of 0.1 M or greater, 0.5 M or greater, or 1.0 M or greater, or 2.0 M or greater. Suitable kosmotropic salts may be present in the isolation solution in an amount of 6.0 M or less, 5.0 M or less, or 4.0 M or less. The kosmotropic salts may be added in an amount ranging from 0.1 M to 6M, 0.5 M to 2.5 M, or 0.5 M to 3.0 M.

Examples of chaotropic salts that may be present in the solution include sodium chloride, calcium chloride, magnesium chloride, and any combination thereof. In some embodiments, the isolation solution includes 1 M or less, 0.5 M or less, or 0.1 M or less of chaotropic salts. In some embodiments, the isolation solution is free or substantially free of chaotropic salts.

Suitable additives include glycerol and other polyols; protease inhibitors; phosphatase inhibitors; cryoprotectants; detergents; chelating agents; reducing agents; and any combination thereof. Suitable additives may be present in the isolation solution in amounts of 0.01 mM or greater, 0.1 mM or greater, 1 mM or greater, 5 mM or greater, 10 mM or greater, or 20 mM or greater. Suitable salts may be added in an amount of 100 mM or less, 50 mM or less, 30 mM or less, 10 mM or less, 5 mM or less, or 1 mM or less. Suitable additives may be present in the isolation solution in amounts ranging from 0.01 mM to 100 mM, 1 mM to 50 mM, 5 mM to 30 mM, 5 mM to 20, 0.01 mM to 5 mM, or 1 mM to 5 mM.

In some embodiments, the carbohydrate binding domain often makes use of a cofactor to bind to the target molecule. In such embodiments, the isolation solution may include the cofactor or multiple cofactors. For example, in embodiments where the carbohydrate binding domain is the carbohydrate binding domain of the lectin or the full lectin protein, one or more cofactors may be included in the isolation media. For examples of lectins that use metal cofactors see Table 4.

The isolation solution solvent may be any solvent that does not degrade or react with the target molecule. In some embodiments, the solvent includes water, an organic solvent, or both. In some embodiments, the includes is an organic solvent such as, for example, methanol, ethanol, isopropanol, and acetonitrile, DMSO, DMF, or any combination thereof. In some embodiments, the majority of the solvent is water. Alternatively, in some embodiments, the majority of the solvent may be made up of organic solvents. In some embodiments, the solvent is nonaqueous, e.g., consists of organic solvents.

The pH of the isolation solution may be any pH that does not make the target molecule unstable or insoluble. Additionally, the pH of the isolation solution should be such that the separation ligands of the separation media are not unstable. The pH of the isolation solution may be controlled to enhance the binding affinity of the target molecules to the affinity groups and/or assistance group (if present).

The isolation solution is contacted with the separation media such that at least a portion of the plurality of the target molecules bind to at least a portion of the separation ligands that include an affinity group and/or an assistance group (if present). Molecules present in the solution that do not include a carbohydrate (i.e., molecules that are not the target molecules), will not bind to the affinity group or will bind to the affinity group with a lesser affinity than the target molecule. Such off target molecules can be removed in a washing step as discussed herein. Through binding to the affinity group, the target molecules are temporarily immobilized on the support substrate.

In some embodiments, the method 300 includes washing the separation media with a washing solution (step 320). Washing the separation media with a washing solution includes contacting the separation media with the washing solution. Washing the separation media may allow for any molecules that are not the target molecule to be removed from the separation media. In the washing step, at least a portion of the target molecules remain bound to the affinity groups and temporarily immobilized on the support substrate.

The washing solution may include a variety of components or may simply be a solvent (e.g., water). The composition and/or pH of the washing solution should be such that none of the components degrade or react with the target molecule. Additionally, the composition and/or pH should be such that the washing solution does not decrease the affinity of the target molecule to the affinity group to a point where the target molecule is able to be removed from the affinity group and washed through the separation media. The washing solution includes a washing solvent. The washing solvent may be water, an organic solvent, or both. The washing solvent may be any solvent as described herein such as those described relative to the isolation solution. In embodiments, the washing solution includes one or more buffering agents, one or more salts, one or more additives, or any combination thereof. In some embodiments, the one or more salts, one or more buffering agents, or one or more additives may be present in the washing solution in any amount as described relative to the isolation solution.

The pH of the washing solution may be any pH that does not make the target molecule unstable or insoluble. Additionally, the pH of the washing solution should be such that the separation ligands of the separation media are not unstable. The pH of the washing solution may be controlled to enhance the binding affinity of the target molecules to the affinity groups and/or decrease the binding affinity of any off target molecules to the affinity groups.

In some embodiments, the washing solution may include a cofactor or multiple cofactors that allow a carbohydrate binding domains (or proteins containing the same) to bind to the target molecule. For example, in embodiments where the carbohydrate binding domain is the carbohydrate binding domain of the lectin or the full lectin protein, one or more cofactors may be included in the washing solution. For examples of lectins that require metal cofactors see Table 4.

In some embodiments, step 320 may be repeated with additional washing solutions. The additional washing solutions may have the same composition and/or pH as the first washing solution or a different composition and/or pH than the first washing composition.

In some embodiments, method 300 further includes eluting the plurality of target molecules that were temporarily immobilized on the support membrane (step 330). The target molecules may be eluted by contacting the separation media with an elution solution. The elution solution includes an elution solvent. The elution solvent may be any solvent as described herein (e.g., the solvent included in the washing solution and/or the isolation solution). The elution solution may be of any composition and/or pH that allows for the target molecules to be separated from the affinity groups and exit the separation media.

In some embodiments, the elution solution may include a molecule that is bound by the affinity group and/or can compete for binding to the affinity group. Such a molecule may be present in an amount such as to compete off the target molecule from an affinity groups. To that end, in some embodiments, the elution solution includes an affinity group competitive molecule and the elution solvent. Different target molecules (or target molecules and other molecules) may be eluted using a linear gradient elution or using a step isocratic elution.

An affinity group competitive molecule is a molecule that binds to the affinity group, and when present at a sufficient concentration can compete off the target molecule from the affinity group. In some embodiments, the affinity group competitive molecule has a higher affinity for the affinity group than the target molecule. In other embodiments, the affinity group competitive molecule has a lower affinity for the affinity group than the target molecule. In yet other embodiments, the affinity group competitive molecule may have the same affinity for the affinity group as the target molecule.

An affinity group competitive molecule may be any molecule that binds to a given affinity group. In some embodiments, an affinity group competitive molecule is a monosaccharide, a disaccharide, or a glycan. In some such embodiments, the affinity group competitive molecule may include the same monosaccharide, disaccharide, or glycosylation pattern as the target molecule glycosylated surface protein. In some embodiments, the affinity group competitive molecule does not include the same monosaccharide or glycosylation pattern as the target molecule. The affinity group competitive molecule may be chosen based on the identity of the affinity group and/or the target. Examples of possible affinity group competitive molecules for affinity groups that include the carbohydrate binding domain of a lectin are shown in Table 5 as the inhibitor or eluting sugar.

An affinity group competitive molecule may be present in an elution solution at a concentration sufficient to compete off the target molecules from the affinity groups. In some embodiments, the affinity group competitive molecule may be present in an elution solution the amount of 20 millimolar (mM) or greater, 50 mM or greater, 100 mM or greater, 200 mM or greater, 300 mM or greater, 400 mM or greater, or 500 mM or greater. In some embodiments, the affinity group competitive molecule may be present in an elution solution the amount of 1 M or less, 500 mM or less, 400 mM or less, 300 mM or less, 200 mM or less, 100 mM or less, or 50 mM or less. In some embodiments, the affinity group competitive molecule may be present in an elution solution the amount of 20 mM to 400 mM, 50 mM to 200 mM, or 100 mM to 500 mM.

In some embodiments where assistance groups are present, the elution solution is of a pH that may decrease the electrostatic and/or hydrophobic interactions between the assistance groups and the target molecule.

In some embodiments, the elution solution includes high amounts of one or more salts in order to decrease the binding affinity between the target molecule and the affinity groups and/or assistance groups (if present). The salt or mixture of salts may be any salt as described herein, for example, in reference to the isolation solution. The salt or mixture of salts may be present in the elution solution in an amount of 50 millimolar (mM) or greater, 100 mM or greater, 150 mM or greater, 200 mM or greater, 300 mM or greater, 500 mM or greater, or 1 M or greater. The salt or mixture of salts may be present in the elution solution in an amount of 5 M or less, 1 M or less, 500 mM or less, 300 mM or less, 200 mM or less, or 100 mM or less.

In some embodiments, the amount and/or identity of a kosmotrope and/or chaotropic salts may be designed to decrease the binding affinity between the target molecules and the affinity groups and/or assistance group (if present).

In some embodiments, the pH of the elution solution may be such as to decrease the binding affinity between the target molecules and the affinity groups. Without wishing to be bound by theory, the pH of the solution may impact the strength and/or availability of various affinity group—target molecule interactions such as hydrogen bonding interactions, electrostatic interactions, hydrophobic interactions, or any combination thereof. In some embodiments, the pH of the elution solution may be higher than the pH of the washing and/or isolation solution. In some embodiments, the pH of the elution solution may be lower than the pH of the washing and/or isolation solution. In some embodiments, the pH of the elution solution may be the same as than the pH of the washing and/or isolation solution.

The volume of the elution solution used to elute the target may vary. For example, in embodiments where the separation media is being employed to concentrate the target, the volume of elution solution is less that the volume of isolation solution.

In some embodiments, the method includes regenerating the separation media. Regeneration is done to prepare the separation media (or the separation media of a separation device) for subsequent uses. Regeneration may include washing the separation media with a solution designed to strip any molecule that is not covalently attached to the support substrate form the separation media. Regeneration may also include flowing an equilibration solution through the separation media such as to prepare the separation media for future use. In some embodiments, the equilibrium solution may be the same as the isolation solution but without the target molecule or the same as the washing solution.

### EXEMPLARY EMBODIMENTS

The following is a non-limiting list of exemplary embodiments according to the present disclosure.

Embodiment 1 is a method of isolating a target molecule from an isolation solution. The isolation solution includes an isolation solvent and the target molecule. The target molecule includes a free protein and the protein includes a carbohydrate. The method includes contacting the isolation solution with a separation media. The separation media includes a support substrate and a plenarily of separation ligands of the formula SL immobilized on the support substrate. The formula SL is

-L-Z (SL)

where L is a linker and Z is a separation group. The separation group includes an affinity group. The affinity group includes a carbohydrate binding domain, a carbohydrate binding ligand, or both.

Embodiment 2 is the method of embodiment 1, where SL is or formula SL1 or SL2.

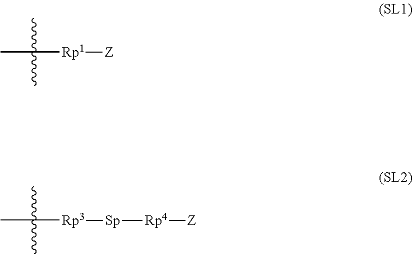

In formula SL1 and SL2 Rp¹, Rp³, and Rp⁴ each independently comprise the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, Rp^{L}, Rp^{Q} or an isomer thereof, wherein Rp^{A}, Rp^{B}, Rp^{C}, Rp^{D}, Rp^{E}, Rp^{F}, Rp^{G}, Rp^{H}, Rp^{I}, Rp^{J}, Rp^{K}, Rp^{L}, and Rp^{M} are represented by:

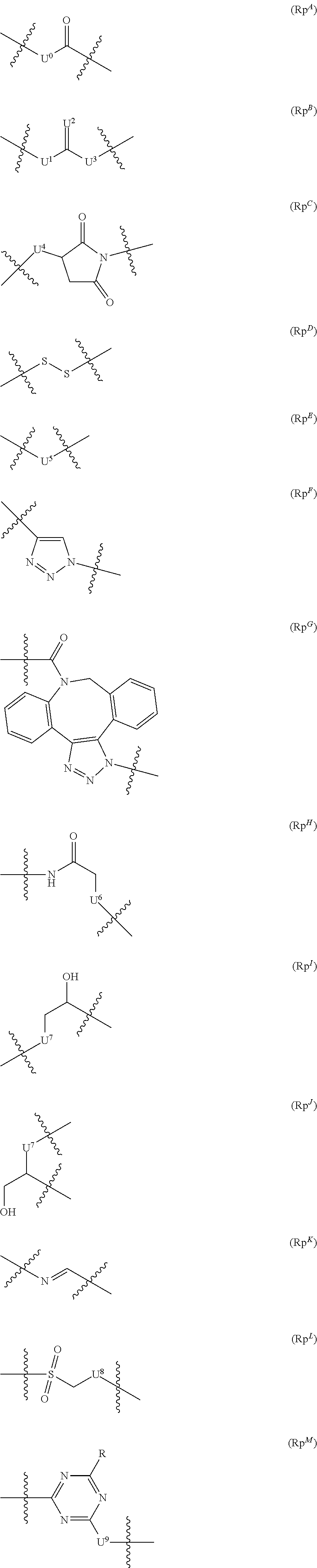

wherein U⁰, U¹, U², U³, U⁴, U⁵, U⁶, U⁷, U⁸ and U⁹ are each independently NH, N, O, or S. Sp is a spacer. R is an organic group, H, or halogen.

Embodiment 3 is the method of embodiment 1 or 2, where U⁰, U¹, U², U³, U⁴, U⁵, U⁶, and U⁷ are each independently NH, O, or S.

Embodiment 4 is the method any one of embodiments 1 to 3, where the plurality of separation ligands are of formula SL2 and Sp is an alkanediyl or alkenediyl comprising one or more catenated functional groups. In some embodiments, the alkanediyl or alkenediyl includes a backbone chain of length C1 to C18.

Embodiment 5 is the method of embodiment 4, where the alkanediyl or alkenediyl includes a backbone chain of length C1 to C3.

Embodiment 6 is the method of any one of embodiment 1 to 5, where the spacer includes —C(O)—.

Embodiment 7 is the method of any one of embodiment 1 to 6, where Rp³, Rp⁴, or both includes Rp^{E}.

Embodiment 8 is the method of any one of embodiment 1 to 6, where Rp³ and Rp⁴ includes Rp^{E}.

Embodiment 9 is the method of embodiment 8, where each U⁵ is O.

Embodiment 10 is the method of embodiment 8, where each U⁵ is NH.

Embodiment 11 is the method of embodiment 8, where one U⁵ is NH and U⁵ is O. In some embodiments, the U⁵ of Rp³ is O and the U⁵ or Rp⁴ is NH. In some embodiments, the U⁵ of Rp³ is NH and the U⁵ or Rp⁴ is O.

Embodiment 12 is the method of embodiment 1 or embodiment 2, where SL2 includes

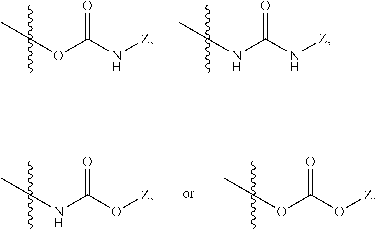

Embodiment 13 is the method embodiment 1 or embodiment 2, where SL2 includes

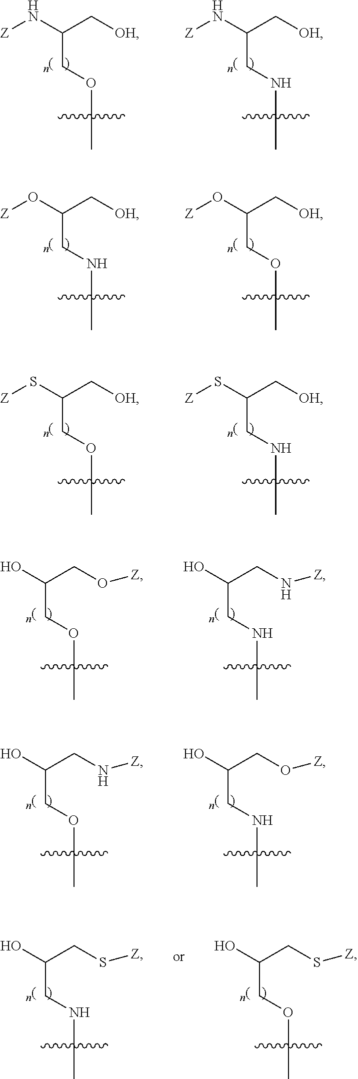

where n is 0, 1, 2, 3, 4, 5, 6, 7, 8, 9, or 10. In some embodiments, n is 1.

Embodiment 14 is the method of any one of embodiments 1 to 13, where the support substrate includes a polyolefin membrane, a polyethersulfone membrane, a poly(tetrafluoroethylene) membrane, a nylon membrane, a fiberglass membrane, a hydrogel membrane, a hydrogel monolith, a polyvinyl alcohol membrane, a cellulose membrane, a cellulose ester membrane, a cellulose acetate membrane, a regenerated cellulose membrane, a cellulosic nanofiber membrane, a cellulosic monolith, a filter paper, or any combination thereof. In some embodiments, the support substrate includes regenerated cellulose membrane.

Embodiment 15 is the method of any one of embodiments 1 to 14, where the method further includes washing the separation media with a washing solution.

Embodiment 16 is the method of any one of embodiments 1 to 15, where the method further includes eluting the target molecule from the separation media with an elution solution.

Embodiment 17 is the method of any one of embodiments 1 to 16, where the isolation solution, the washing solution, the elution solution, or any combination thereof includes an organic solvent.

Embodiment 18 is the method of any one of embodiments 1 to 17, where the isolation solution, the washing solution, the elution solution, or any combination thereof includes water.

Embodiment 19 is the method of any one of embodiments 1 to 18, where the carbohydrate of the target molecule includes sialic acid.

Embodiment 20 is the method of embodiment 19, where the sialic acid is α2,6 linked sialic acid.

Embodiment 21 is the method of embodiment 19, where the sialic acid is α2,3 linked sialic acid.

Embodiment 22 is the method of any one of embodiments 1 to 21, where the carbohydrate of the target molecule includes fucose.

Embodiment 23 is the method of any one of embodiments 1 to 22, where the target molecule includes an antibody or a fragment thereof.

Embodiment 24 is the method of any one of embodiments 1 to 23, where the target molecule includes a first carbohydrate and a second carbohydrate.

Embodiment 25 is the method of embodiments 24, where the first carbohydrate and the second carbohydrate are different.

Embodiment 26 is the method of any one of embodiments 24 to 25, where the first carbohydrate includes α2,6 linked sialic acid and the second carbohydrate includes α2,3 linked sialic acid.

Embodiment 27 is the method of any one of embodiments 1 to 26, where the target molecule includes fetuin or a fragment thereof, mucin or a fragment thereof, asialofetuin or a fragment thereof, or any combination thereof.

Embodiment 28 is the method of any one of embodiments 1 to 27, where the elution solution includes an affinity group competitive molecule.

Embodiment 29 is the method of any one of embodiments 1 to 28, wherein the separation media has a static binding capacity of 0.01 milligrams of target per 1 mL of bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media or separation device containing the same has a static binding capacity of 150 mg/mL bed volume or less, 120 mg/mL bed volume or less 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a static binding capacity of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume.

Embodiment 30 is the method of any one of embodiments 1 to 29, wherein the separation media has a dynamic binding capacity at 10% breakthrough of 0.01 milligrams of target per 1 mL bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media has a dynamic binding capacity at 10% breakthrough of 150 mg/mL bed volume or less, 120 mg/mL bed volume or less 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a dynamic binding capacity at 10% breakthrough of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume.

Embodiment 31 is the method of any one of embodiments 1 to 30, separation ligand density of 0.01 milligrams of separation ligand per 1 mL of bed volume (mg/mL bed volume) or greater, 0.1 mg/mL bed volume or greater, 1 mg/mL bed volume or greater, 5 mg/mL bed volume or greater, 10 mg/mL bed volume or greater, 20 mg/mL bed volume or greater, 30 mg/mL bed volume or greater, 40 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, 110 mg/mL bed volume or greater, or 120 mg/mL bed volume or greater. In some embodiments, a separation media has a separation ligand density of 150 mg/mL bed volume or less, 120 mg/mL bed volume or less, 110 mg/mL bed volume or less, 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 30 mg/mL bed volume or less, or 20 mg/mL bed volume or less, 10 mg/mL bed volume or less, 5 mg/mL bed volume or less, 1 mg/mL bed volume or less, or 0.1 mg/mL bed volume or less. In some embodiments, a separation media has a separation ligand density of 0.01 mg/mL bed volume to 150 mg/mL bed volume, 0.1 mg/mL bed volume to 150 mg/mL bed volume, 1 mg/mL bed volume to 150 mg/mL bed volume, 5 mg/mL bed volume to 150 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 10 mg/mL bed volume to 20 mg/mL bed volume, 15 mg/mL bed volume to 60 mg/mL bed volume, 15 mg/mL bed volume to 50 mg/mL bed volume, 15 mg/mL bed volume to 30 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 20 mg/mL bed volume to 30 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, or 1 mg/mL bed volume to 10 mg/mL bed volume.

Embodiment 32 is the method of any one of embodiments 1 to 31, wherein the separation media has a separation specific surface area (SSA) of 1.5 meters squared per milliliter of bed volume (m²/mL bed volume) or greater, 2 m²/mL bed volume or greater, 3 m²/mL bed volume or greater, 4 m²/mL bed volume or greater, 5 m²/mL bed volume or greater, 8 m²/mL bed volume or greater, 9 m²/mL bed volume or greater, 10 m²/mL bed volume or greater, or 15 m²/mL bed volume when the support substrate has an average pore size of 0.1 μm to 10.0 μm, such as 0.2 μm to 0.5 μm. In some embodiments the separation media has a separation SSA of 20 m²/mL bed volume or less, 15 m²/mL bed volume or less, 10 m²/mL bed volume or less, 9 m²/mL bed volume or less, 8 m²/mL bed volume or less, 7 m²/mL bed volume or less, 6 m²/mL bed volume or less, 5 m²/mL bed volume or less, 4 m²/mL bed volume or less, or 3 m²/mL bed volume or less, 2 m²/mL bed volume or less when the support substrate has an average pore size of 0.1 μm to 10.0 μm, such as 0.2 μm to 0.5 μm. In some embodiments the separation media has a separation SSA of 1.5 m²/mL bed volume to 20 m²/mL bed volume, 1.5 m²/mL bed volume to 15 m²/mL, 1.5 m²/mL bed volume to 10 m²/mL, 2 m²/mL bed volume to 20 m²/mL, 2 m²/mL bed volume to 15 m²/mL, 2 m²/mL bed volume to 10 m²/mL, 2 m²/mL bed volume to 9 m²/mL, 2 m²/mL bed volume to 8 m²/mL, 2 m²/mL bed volume to 7 m²/mL, 2 m²/mL bed volume to 6 m²/mL, 2 m²/mL bed volume to 5 m²/mL, 3 m²/mL bed volume to 20 m²/mL, 3 m²/mL bed volume to 15 m²/mL, 3 m²/mL bed volume to 10 m²/mL, 4 m²/mL bed volume to 20 m²/mL, 4 m²/mL bed volume to 15 m²/mL, 4 m²/mL bed volume to 10 m²/mL, 5 m²/mL bed volume to 20 m²/mL, 5 m²/mL bed volume to 15 m²/mL, or 5 m²/mL bed volume to 10 m²/mL when the support substrate has an average pore size of 0.1 μm to 10.0 μm, such as 0.2 μm to 0.5 μm.

Embodiment 33 is the method of any one of embodiments 1 to 32, wherein the separation media has an average pore size of 10 μm or less, 5 μm or less, 2 μm or less, 1 μm or less, 0.6 μm or less, 0.5 μm or less, 0.45 μm or less, or 0.2 μm or less. The membrane may have an average pore size of 0.1 μm or greater, 0.2 μm or greater, 0.45 μm or greater, 0.5 μm or greater, 0.6 μm or greater, 0.7 μm or greater, or 1 μm or greater. The membrane may have an average pore size ranging from about 0.1 μm to 10.0 μm, 0.1 μm to 0.2 μm, 0.1 μm to 0.45 μm, 0.1 μm to 0.5 μm, 0.1 μm to 1 μm, 0.2 μm to 0.45, 0.2 μm to 0.50, 0.2 μm to 1 μm, 0.2 μm to 2 μm, 0.2 μm to 10 μm, 0.45 μm to 1 μm, 0.45 μm to 2 μm, 0.45 μm to 10 μm, 1 μm to 2 μm, or 1 μm to 5 μm. In some embodiments, the support substrate has an average pore size of 0.1 μm to 0.5 μm, 0.1 μm to 0.6 μm, 0.1 μm to 0.3 μm, or 0.4 μm to 0.6 μm.

### EXAMPLES

These examples are merely for illustrative purposes only and are not meant to be limiting on the scope of the appended claims. All parts, percentages, ratios, etc. in the examples and the rest of the specification are by weight, unless noted otherwise.

In the Examples, pore sizes are understood to be average pore sizes.

### Example 1: Example Synthetic Methods to Prepare the Separation Media of the Present Disclosure

Several synthetic strategies may be employed to construct the separation media of the present disclosure. The synthetic strategies include direct or indirect immobilization of separation ligands. The synthetic strategies also include amine assisted coupling or organic solvent assisted coupling.

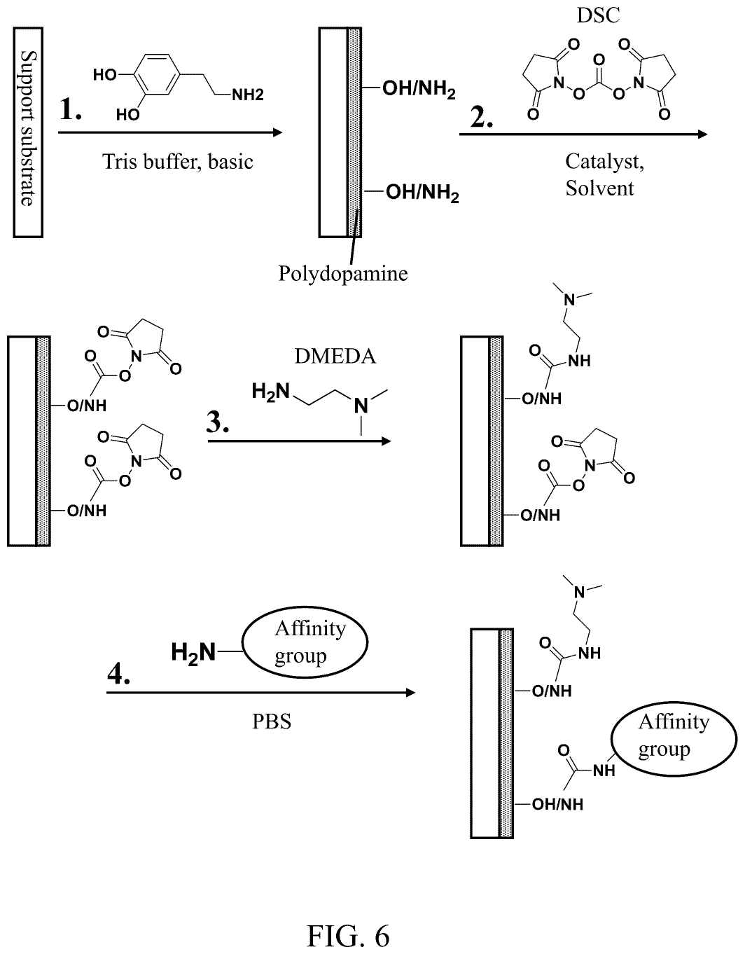

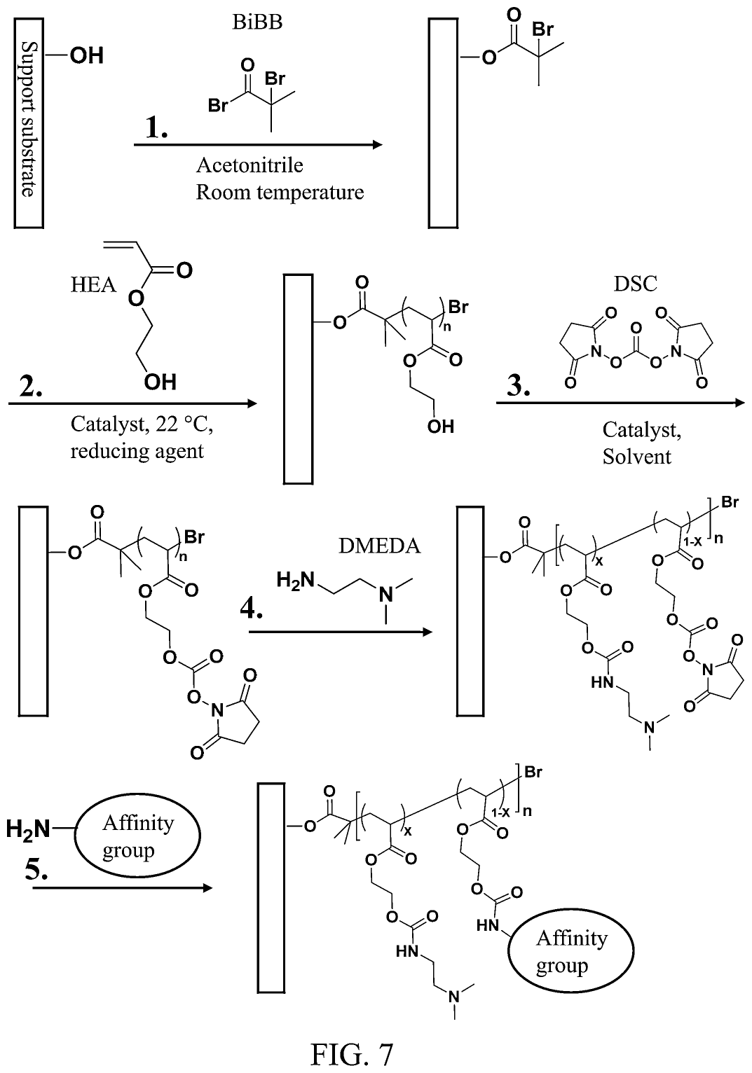

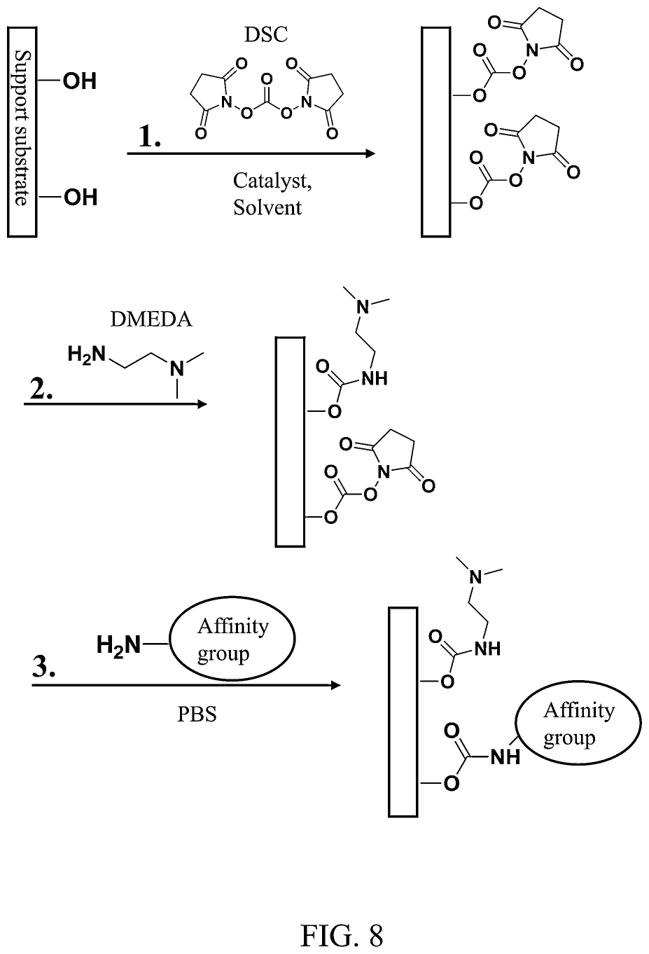

FIG. 6 and FIG. 7 show schematics of synthetic schemes that include the indirect immobilization of the separation ligands to the support substrate. The strategies of FIG. 6 and FIG. 8 also include the amine assisted coupling method.

In Step 1, of the scheme in FIG. 6, polydopamine (PDA) is incorporated onto a support membrane through oxidative polymerization of dopamine in basic aqueous buffer in the presence of air. Deposition of PDA may function to hydrophilize the support substrate and introduce support substrate reactive handles (e.g., hydroxyl, amine, and quinone). In step 2, the PDA reactive handles may be reacted with the bifunctional (has two NHS ester reactive handles) of linker precursor disuccinimidyl carbonate (DSC). The PDA reactive handles (OH and NH₂) may react with the first N-hydroxy succinimidyl (NHS) ester of the linker precursor to form a first amide or carbamate reaction product. In step 3, a portion of the second NHS ester groups may be reacted with the amine reactive handle of N,N-dimethylethylenediamine (DMEDA; a first separation group precursor) to form a second amide reaction product. The DMEDA groups (e.g., the tertiary amine of DMEDA) may act as cationic assistance affinity groups to increase the local concentration of negatively charged (in aqueous buffer) affinity groups at the membrane surface through coulombic interaction. In step 4, the second portion of the second NHS ester groups may be reacted with a second separation group (including an affinity group) precursor reactive handle (an amine as shown in this scheme) to form an amide reaction product. The conjugation reaction of step 4 may be done in an aqueous buffer.

In the first step of the scheme depicted in FIG. 7, an initiator (α-bromoisobutyryl bromide, BiBB) may be coupled to the OH groups of a support substrate. In the second step, hydroxyethyl acrylate monomers may be polymerized from the immobilized initiator to from poly(HEA). In step 3, the poly(HEA) reactive handles (OH groups) may be reacted with the bifunctional (has two NHS ester reactive handles) linker precursor disuccinimidyl carbonate (DSC). The poly(HEA) reactive handles (OH) may react with the first NHS ester of the linker precursor to form a first carbamate reaction product. In step 4, a portion of the second NHS ester groups may be reacted with the amine reactive handle of N,N-dimethylethylenediamine (DMEDA; a first separation group precursor) to form an amide reaction product and install the separation ligands containing the amine assistance groups. The DMEDA groups (e.g., the tertiary amine of DMEDA) may act as cationic assistance groups which may allow for a higher density of negatively charged (in aqueous buffer) affinity groups at the support substrate surface through coulombic interaction. In step 5, the second portion of the second NHS ester groups may be reacted with a second separation group (including the affinity group) precursor reactive handle to form an amide reaction product and install the separation ligands containing the affinity group on the support substrate. The conjugation reaction of step 5 may be completed in an aqueous buffer reaction mixture. In some embodiments, the support substrate may be exposed to a tris base solution as a final step to quench unreacted NHS intermediates and to install separation ligands containing a capping group.

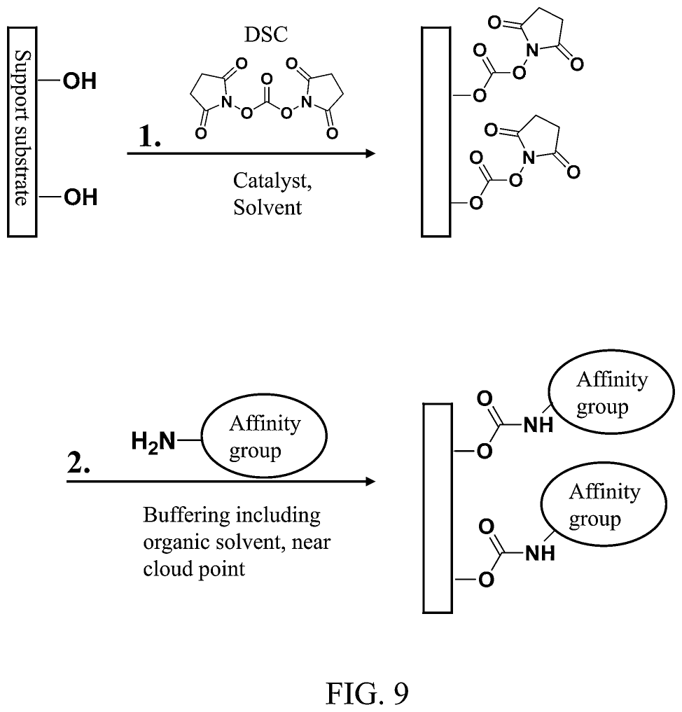

FIG. 8 and FIG. 9 show schematics of synthetic schemes that include the direct immobilization of the separation ligands on the support substrate. The strategy of FIG. 8 includes the amine assisted cooling method. The strategy of FIG. 9 includes the organic solvent assistance method.

FIG. 8 shows a synthetic strategy where the separation ligands can be directly immobilized on the support substrate and the amine assisted method may be used to achieve a high density of separation ligands. This synthetic strategy is similar to the strategy in FIG. 7 except that the membrane was not functionalized with a polymer. Instead, the hydroxyl reactive handles of the support substrate can be directly reacted with one of the NHS ester reactive handles of DSC to form a carbamate reaction product (step 1). Separation ligands having an amine assistance group can be installed (step 2). The amine assistance group may facilitate the installation of the separation groups that include an affinity group (step 3). The conjugation reaction of step 3 may be done in an aqueous buffer reaction mixture.

FIG. 9 shows a synthetic strategy where the separation ligands can be directly immobilized on the support substrate and the organic solvent assisted method may be used to achieve a high density of separation ligands. Residual tertiary amine moieties in the final separation media may have the potential for nonspecific binding when the solution conductivity is very low. As affinity chromatography typically is performed at conductivity levels above that which tertiary amines retain significant binding capacity, the residual amine groups were expected to have negligible effect on chromatographic performance. In an effort to circumvent this potential issue completely, an organic solvent assisted coupling method may be employed to install the separation ligands containing the affinity group. The organic assisted coupling method utilizes water-miscible organic solvents as a constituent of the coupling solution to increase separation group precursor coupling efficiency, which enables use of low separation group precursor concentrations in the coupling solution. Additions of organic solvents to the aqueous buffered reaction mixture (10%-80% by volume depending on the organic solvent used) to bring solution near the cloud point. At the cloud point, the reaction mixture starts to appear turbid upon increasing the concentration of organic solvent. Organic solutions replace water molecules in the separation group precursor solvation shell which can facilitate greater interaction between the separation group precursor and the support substrate. Additional organic solutions added beyond the cloud point may exacerbate aggregation and flocculation dynamics of the separation group precursor, which can comparatively reduce efficiency of coupling reaction. This coupling methodology may allow for high performance separation media to be prepared with low separation group consumption.

FIG. 9 shows an example synthetic scheme that may be used to prepare separation media of the present application via direct immobilization of the separation ligands using the organic solvent assisted coupling method. In step 1 the support substrate reactive handles (OH) may be reacted with the first N-hydroxy succinimidyl (NHS) ester of the DSC linker precursor to form a carbamate reaction product. In the second step, a reaction mixture that is near the cloud point that includes the separation group precursor (includes the affinity group), water, and a water-miscible solvent may be exposed to the reaction product of step 2. The second NHS ester groups of the support substrate-linker conjugate may react with the reactive handle (NH₂) of the separation group precursor to form an amide reaction product and install the separation ligands having the affinity group on the support substrate.

### Example 2: Assessment of Synthetic Methods to Prepare Separation Media

Two “amine assisted method” synthetic techniques were explored to install the support membrane reactive handles of the separation media on a base membrane. Without wishing to be bound by theory, it was thought that the incorporation of amine assistance groups may allow for a high density of negatively charged lectin affinity groups to be immobilized on the separation media.

The synthetic scheme of FIG. 6 was used to construct some of the separation medias of Example 2. The separation media made according to FIG. 6 included separation ligands having a lectin affinity group and separation ligands having an ionizable group (the tertiary amine of DMEDA). The scheme includes the polymer indirect immobilization of the separation ligands through the amine assisted method. In step 1, polydopamine (PDA) was incorporated into a polypropylene (PP) macroporous membrane through oxidative polymerization of dopamine in basic aqueous buffer in the presence of air. Deposition of PDA was intended to hydrophilize the PP membrane and introduce support substrate reactive handles (e.g., hydroxyl, amine, and quinone). The next step was to react the PDA reactive handles with the bifunctional (has two NHS ester reactive handles) linker precursor disuccinimidyl carbonate (DSC). The PDA reactive handles (OH and NH₂) react with the first N-hydroxy succinimidyl (NHS) ester of the linker precursor to form a first amide or carbamate reaction product. In step 3, a portion of the second NHS ester groups are reacted with the primary amine reactive handle N,N-dimethylethylenediamine (DMEDA) to form a second amide reaction product. The DMEDA groups (e.g., the tertiary amine of DMEDA) act as cationic assisting groups to increase the local concentration of negatively charged (in aqueous buffer) lectin molecules at the membrane surface through coulombic interaction. In step 4, the second portion of the second NHS ester groups are reacted with the lectin amine reactive handle to form an amide reaction product.

Reaction conditions for the PDA deposition step (step 1) of FIG. 6 were evaluated. The goal was to achieve a high degree (reflected by mass gain) of uniform, irreversible coating of the membrane pores. Dopamine concentration, buffer composition, reaction time, temperature, and membrane type (PP or regenerated cellulose (RC)) were tested. The results are summarized in Table 6. In each condition, the dopamine solution turned dark green-brown, and eventually a polymer-like precipitate formed, indicating polymerization of dopamine. In fully aqueous deposition buffer, the hydrophobic PP membrane turned dark brown, but gained negligible mass. The hydrophilic RC membrane gained a very small amount of mass. The addition of 20% or 50% ethanol facilitated wetting of the PP membrane, but mass gain was still low. Additionally, leaching of PDA was observed upon rinsing and modification of the membranes in step 2 of FIG. 6.

TABLE 6
Polydopamine deposition conditions and results

| | Deposition | Membrane | Mass |
|---|---|---|---|
| PDA deposition solution | temperature, time | base, pore size | gain |
| 2 mg/mL dopamine, 10 mM Tris pH 8.5 | 22° C., 4 h | PP, 1.2 μm | none |
| 2 mg/mL dopamine, 10 mM Tris pH 8.5 | 22° C., 4 h | RC, 1 μm | 0.9% |
| 6 mg/mL dopamine, 10 mM Tris pH 8.5 | 22° C., 4 h | RC, 1 μm | 0.6% |
| 6 mg/mL dopamine, 10 mM Tris pH 8.5, | 22° C., 20 h; followed | PP, 3 μm | 1.4% |
| 20% ethanol | by 40° C., 5 h | | |
| 8 mg/mL dopamine, 10 mM Tris pH 8.5, | 22° C., 17 h; followed | PP, 3 μm | 1.3% |
| 50% ethanol | by 40° C., 5 h | | |

To check for PDA leaching and low functionalization, RC membranes were chosen for direct immobilization of the separation ligands using the amine assisted method for making separation media. FIG. 7 shows an amine assisted method used to construct some of the separation medias of Example 2. The direct immobilization assisted amine method was similar to the polymer amine assisted method except that the membrane was not functionalized with a polymer. Instead, the hydroxyl reactive handles of the membrane were directly reacted with one of the NHS ester reactive handles of DSC to form a carbamate reaction product.

### Example 3: Evaluation of Separation Media for Static Binding Capacity (SBC)

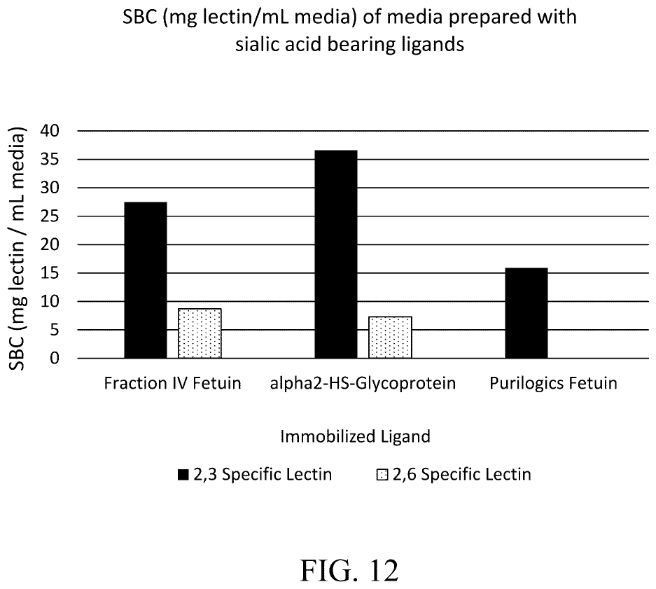

Separation media were prepared according to the indirect immobilization of the separation ligands through the amine assisted method discussed in Example 1 and 2 (“Amine Assisted” in FIG. 13) and using direct immobilization of the separation ligands without the amine assisted method (similar to FIG. 8 but without step 2; see FIG. 9; “Direct” in FIG. 13); that is media prepared in Example 3 by the direct immobilization did not include DMEDA (the cationic assistance group; step 2 of FIG. 8). The separation medias were prepared by reacting the NHS ester reactive handles of the DSC already immobilized to the support membrane with different concentrations of Concanavalin A (Con A; 2 mg/ml, 5 mg/ml, 12 mg/ml, 25 mg/ml, and 45 mg/ml) and in reaction mixtures having different pH values (7, 7.25, 7.5, and 8).

To determine static binding capacity (SBC) of the separation media, a model glycoprotein, porcine thyroglobulin (PTG), was used. PTG is a model glycoprotein used to report the binding capacity of commercial Concanavalin A resins. Although it is relatively large for a protein (670 kDa, hydrodynamic diameter=17 nm), it is still much smaller than lentiviral vectors (diameter=100 nm). PTG was used as a probe to validate separation ligand coupling to the base membrane (of the support substrate) and to rapidly screen synthetic conditions. SBCs were measured by mass balance using the difference between initial and equilibrium (2 h) porcine thyroglobulin concentrations measured by UV spectroscopy. The binding condition for each separation media with PTG was 2 mg/mL PTG in 20 mM Tris at pH 7.4, 0.5 M NaCl, 1 mM MnCl₂, and 1 mM CaCl₂). The purpose of the 0.5 M NaCl was to screen non-specific electrostatic interactions so that only binding of the target molecule to the affinity group is observed.

FIG. 13 shows the SBC for the separation media tested. Binding capacity had a weak dependence on Con A concentration in the coupling step (the coupling of the separation ligand including the lectin affinity group). Increasing the Con A concentration from 5 to 45 mg/mL increased the SBC only slightly from 32 to 38 mg PTG/mL membrane. At a fixed Con A concentration, the indirect immobilization of the separation ligands through the amine-assisted method resulted in slightly higher SBCs than the direct immobilization of the separation ligands without the amine assisted method. pH 7.75 was observed to be the most effective coupling, which resulted in an SBC of 36 mg/mL when a Con A coupling concentration of 12 g/mL was used.

### Example 4: Assessment of Additional Synthetic Methods to Prepare Separation Media

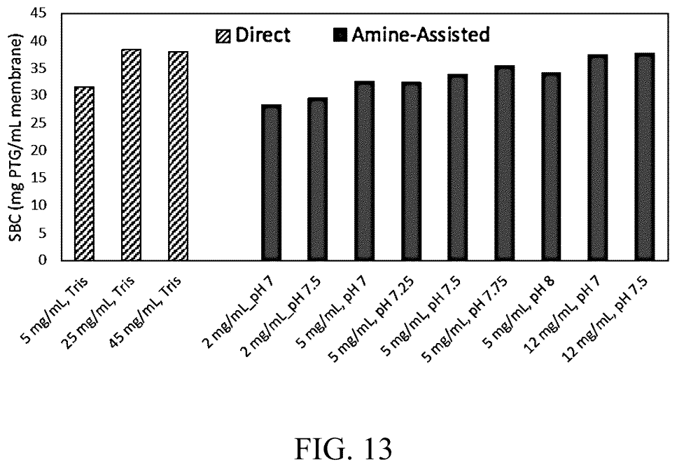

In an effort to increase the binding capacity of the separation media, direct immobilization of the separation ligands (including a lectin affinity group) using an organic-solvent assisted coupling method was explored (FIG. 9, “Solvent-assisted” in FIG. 14). In this method, during the affinity group coupling step (step 2; coupling of lectin), a water-miscible organic solvent (e.g., ethanol) was added to the lectin coupling solution to bring the solution near the cloud point. Different buffer types used during step 2 (e.g., carbonate and phosphate) and a commonly used “salt-out” method were also explored (“Salt-out” in FIG. 14).

FIG. 14 shows the SBC for the organic solvent assisted (solvent-assisted) method with various buffers, the salting out method, and the direct immobilization of the separation ligands using an organic-solvent assisted technique. Pore size of the support substrate membrane was also varied. Using a Con A coupling concentration of 5 mg/mL the organic solvent assisted method results in an SBC of 37 mg PTG/mL, which is similar to that obtained using either the direct method or the amine-assisted method with the 0.2 μm pore size membrane. In some embodiments, the direct immobilization of the separation ligands using the organic solvent assisted method is preferred over the amine assisted method because it achieves both high binding capacity using low protein concentration and is also expected to have low nonspecific binding due to the absence of tertiary amine assistance groups.

The direct immobilization of the separation ligands using an organic-solvent assisted technique was explored for larger pore sizes. Using this synthetic technique, the SBC decreased to 25 mg/mL and 15 mg/mL for pore sizes of 0.45 μm and 1.0 μm respectively. This is likely due to a reduction in specific surface area upon increasing the pore size. Even though the capacity is lower, larger pore size may exhibit increased performance for capturing the large lentiviral vectors.

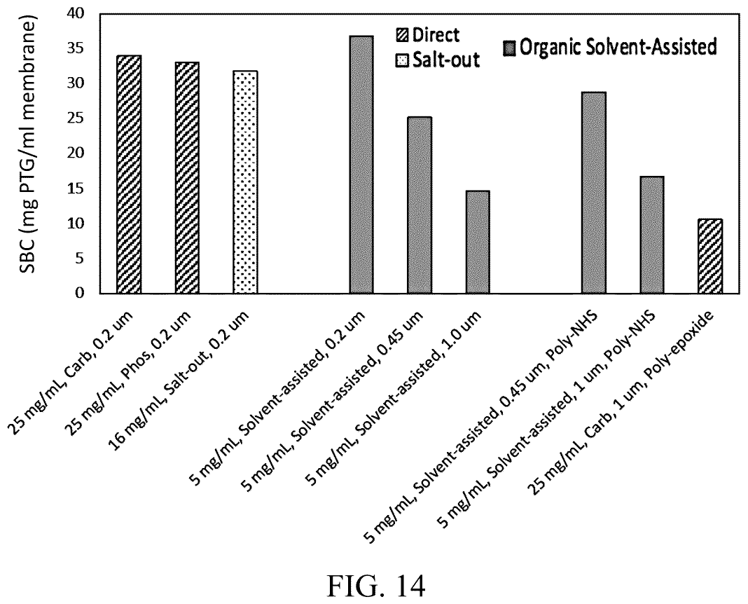

In an effort to increase binding capacity for the larger pore size membranes, an indirect immobilization of the separation ligand method was tested. In this method, a polymer was grafted from the surface membrane to install the support substrate reactive handles. This method is different from the polydopamine polymer deposition in Example 2 because the polymer was grown from the substrate surface (i.e., grafting from) not grafted onto the substrate surface. FIG. 15 shows the polymer grafting from the support surface synthetic scheme employed. Initiator moieties were coupled to a RC membrane (step 1) followed by polymer grafting via surface-initiated activators generated by electron transfer atom transfer radical polymerization (step 2; SIAGET ATRP). In step 1, the initiator 2-bromo-2-methylpropionyl bromide (BiBB) was coupled to the OH groups of the membrane surface. In step 2, 2-hydroethylacryalte (HEA) was polymerized from the surface initiator. The method may be used to prepare other functional poly(meth)acrylates, such as poly(acrylic acid) (PAA) and poly(glycidyl methacrylate) (PGMA) on the support substrate. Without wishing to be bound by theory, it is thought that grafting from the substrate surface to include flexible polymer chains will both increase the number of potential separation ligand coupling sites and reduce steric hinderance for the binding of the large species to the separation ligands, such as lentiviral vectors.

PolyHEA was grafted from RC membranes with pore sizes of 0.45 μm and 1.0 μm. Con A (5 mg/mL) was immobilized using the organic solvent assisted method and DSC (poly-NHS in FIG. 14). For both pore sizes the SBC increased slightly upon polymer grafting, going from 25 to 29 mg/mL for 0.45 μm and from 15 to 17 mg/mL for 1 μm.

PolyGMA-grafted was grafted from a membrane having a pore size of 1 μm membrane and Con A (25 mg/mL) was coupled without using the organic assistance method (poly-epoxide in FIG. 14). This separation media resulted in an SBC of 11 mg/mL.

### Example 5: Affinity Group Library Assessment and Rationale

In addition to Con A, four other lectins were evaluated as potential affinity ligands (affinity groups): Galanthus nivalis lectin (GNL), Musa paradisiaca lectin (BanLec), Triticum vulgaris (wheat germ) lectin (WGA), and Maackia amurensis lectin (MAL-I). Like Con A, GNL and BanLec are glucose and/or mannose binding lectins. WGA and MAL-I are sialic acid binding lectins and are expected to bind VSV-G through its terminal glycan residues, which contain both mannose and sialic acid.

In addition to protein lectin affinity groups, nine carbohydrate binding ligands (small molecules) were evaluated as potential affinity groups for lentiviral vector purification. The carbohydrate binding ligands were selected based on reports in the literature of binding similar proteins as VSV-G or inhibiting viruses of various types, or they have a chemical structure similar to those reported. Some of the chemical structures resemble certain aspects of heparin, a known animal-derived affinity group for lentiviral vectors. They were explored as potential heparin “mimics”. Other ligands are based of boronate affinity groups, which reversibly bind cis diols in glycoproteins. The library and the rationale for each potential ligand is summarized in Table 7.

TABLE 7
Carbohydrate binding ligands tested

| | | | Linker |
|---|---|---|---|
| Ligand | Type | Rationale | Chemistries Used |
| Concanavalin A | Lectin; binds | May bind mannose and | NHS |
| (Con A) | mannose and glucose | glucose units of VSV-G | |
| Galanthus nivalis | Lectin; binds | May bind mannose units of | NHS |
| lectin (GNL) | mannose | VSV-G | |
| Musa paradisiaca | Lectin; binds | May bind mannose and | NHS |
| lectin (BanLec) | mannose and glucose | glucose units of VSV-G | |
| Triticum vulgaris | Lectin; binds GlcNAc | May bind terminal sialic | NHS |
| (wheat germ) | and sialic acid | acid units of VSV-G | |
| lectin (WGA) | | | |
| Maackia | Lectin bind; binds | May bind terminal sialic | NHS |
| amurensis lectin | sialic acid with a-2,3- | acid units of VSV-G with a- | |
| (MAL-I) | Gal-b-1,4-GlcNAc | 2,3-Gal-b-1,4-GlcNAc | |
| | linkage | linkage | |
| Thiosalicylic acid | Multimodal, cation | May mimic heparin | Poly-epoxide |
| (TSA) | exchange | structure | |
| 4-Mercaptophenyl | Boronate affinity | Reversibly binds cis-diols | Poly-epoxide |
| boronic acid | | of glycoproteins | |
| (MPBA) | | | |
| 4-Mercaptophenol | Phenol | Structure is similar to | Epoxide, Poly- |
| (MPhOH) | | tannins | epoxide |
| Gallic acid | Polyphenol | Structure is similar to | Succinic-DCC, |
| | | tannins | Polyacrylic-DCC |
| Serotonin | Phenol, multimodal | Reported to bind sialic acid | NHS, Poly-NHS |
| | | containing proteins | |
| Epigallocatchin | Polyphenol, tannin | This tannin in green tea is | Succinic-DCC, |
| gallate (EGCG) | | reported to bind/inhibit | Polyacrylic-DCC |
| | | viruses | |
| Reactive Red 120 | Dye affinity, | May mimic heparin | Triazine, Poly- |
| | multimodal | structure | triazine |
| Cibracon Brilliant | Dye affinity, | May mimic heparin | Triazine |
| Yellow 3G-P | multimodal | structure | |
| Reactive Blue 4 | Dye affinity, | May mimic heparin | Triazine |
| | multimodal | structure | |

### Example 6: Performance Evaluation of Separation Media Using Vector Cell Supernatant

Affinity membrane column prototypes were evaluated for performance using virus vector cell supernatant (isolation solution) that included the target molecule (viral vectors). VIVIDCOLORS pLenti6.3/V5-GW/EmGFP Expression Vector was used in these studies. The viral vectors were produced by transfecting pLenti6.3/V5-GW/EmGFP into 293FT (R700-07) cells using the VIRALPOWER Supporting kit (K4970-00). 293FT cells were grown in DMEM cell culture medium supplemented with 10% FBS, 0.1 mM non-essential amino acids, 6 mM L-glutamine, 1 mM MEM sodium pyruvate and 500 μg/ml geneticin. The 293FT cell line is a suitable host for lentiviral production. The 293FT cell line was derived from the 293F cell line and stably expressed the SV40 large T antigen from the pCMVSPORT6TAg.neo plasmid. The envelope protein on the surface of the viral vectors was VSV-G glycoprotein.

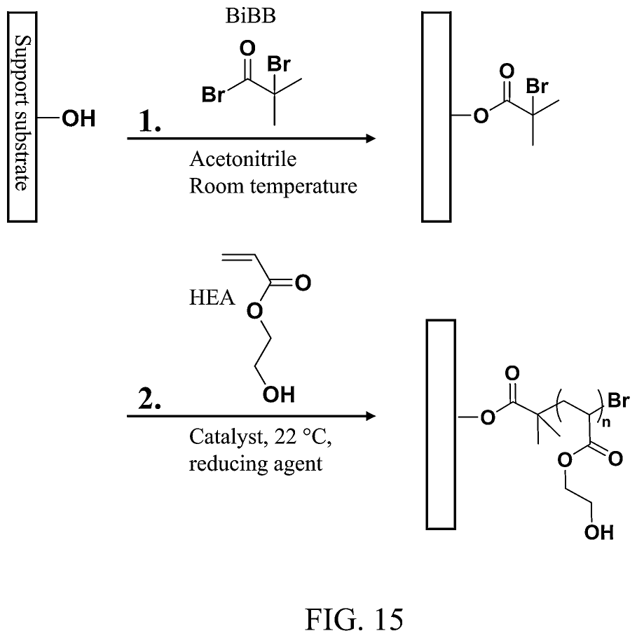

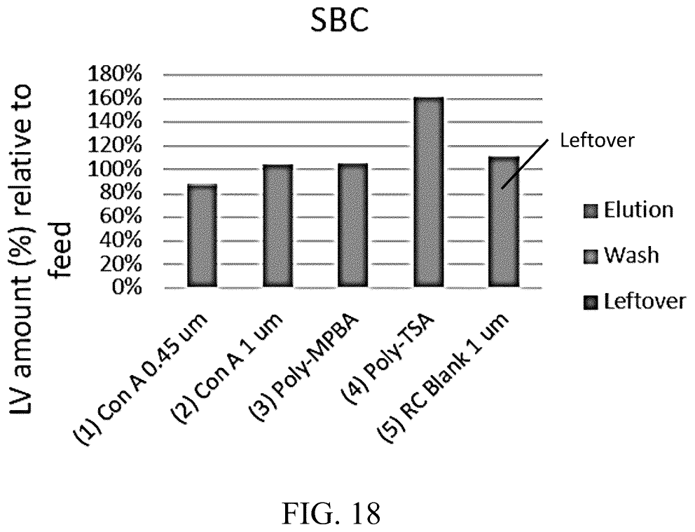

Bind-and-elute tests with LV cell culture supernatant (not purified) were used for initial evaluation of the membrane column prototypes. Note: The bind-and elute buffer conditions were not optimized in these studies; these tests were used to rapidly screen a large number of potential separation ligands. FIG. 16 shows the results of dynamic lentiviral vector bind-and-elute tests with Con A affinity membranes prepared by the organic solvent assisted method with pore sizes of 0.45 μm and 1.0 μm. “Leftover” in FIG. 18 refers to the flowthrough. Columns packed with “blank” membranes (i.e. membranes without immobilized separation ligands), were tested for comparison. The flow rate was 0.5 mL/min (3 sec residence time). For samples 1, 2, 4, and 5, the lentiviral vector sample was loaded directly from the culture media (2.5 mL, 3.68×105 viral particles/mL). For samples 3 and 6, the lentiviral vector media was buffer exchanged to PBS prior to loading (2.5 mL, 1.18×10⁶ viral particle/mL). The wash buffer was 20 mM Tris at pH 7.4, 0.5 M NaCl (3 mL). The elution buffer was 20 mM Tris at pH 7.4, 0.5 M NaCl, with 0.3 M methylglucoside (2 mL). The amount of lentiviral vector was quantified in the feed, flowthrough, wash, and elution fraction by PCR. For the Con A modified columns, the amount of lentiviral vectors in the flowthrough and wash samples was slightly decreased compared to the blank membrane controls. However, very little lentiviral vector was observed in the elution fractions. To detect virus transduction activity in the fractions, 0.1 mL samples were added to HEK293 cells with 0.1 mL medium and polybrene. The lentiviral vector and cells were incubated overnight, and observed under fluorescence microscope 72 h after transduction. Green cells indicate transduction with GFP. The presence of GFP positive cells in the flowthrough transduction sample indicates that an insignificant amount of active virus was bound to the column. No GFP positive cells were found in the wash or elution fractions.

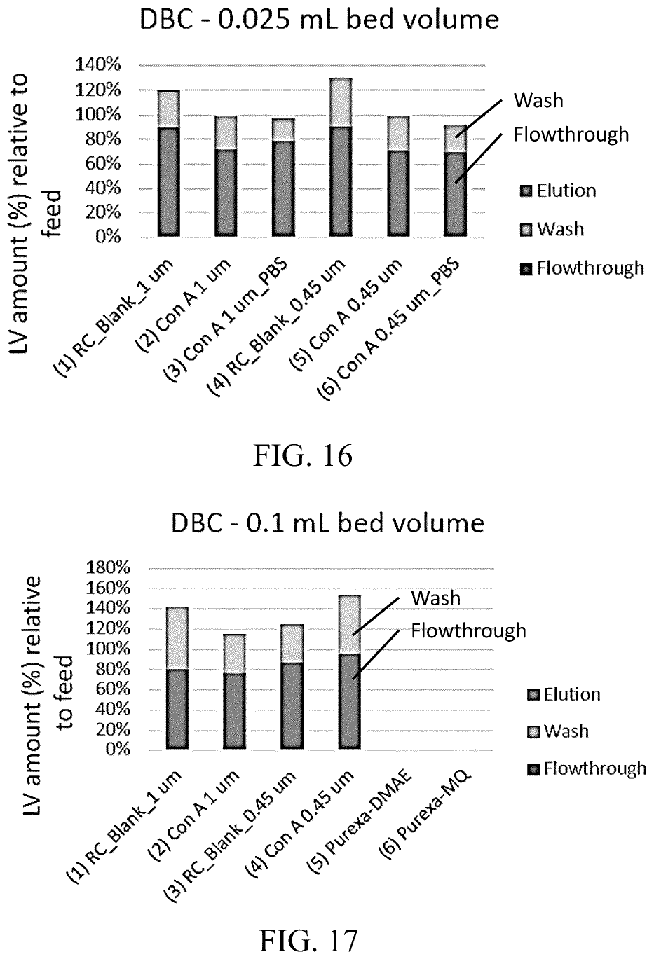

For the next round of study, the bed volume was increased by four times from 0.025 mL to 0.1 mL for Con A membranes (bed volume) with average pore sizes of 0.45 μm and 1.0 μm. The same flow rate (0.5 mL/min) and rinse and elution buffers were used. Lentiviral vector cell supernatant (2 mL, 7.22×10⁵ viral particles/mL) was loaded onto the columns, and the lentiviral vector titer in each fraction was determined by PCR. Similar to the previous experiment, the amount of lentiviral vector in the flowthrough and wash was decreased slightly for the Con A functionalized membranes compared to the blank controls and very little elution was observed (FIG. 17). For comparison, the same amount of lentiviral vector supernatant was loaded onto weak anion-exchange (Sample 5, PUREXA-DMAE) and multimodal strong anion-exchange (Sample 6, PUREXAMQ) columns (FIG. 17). The wash buffers were 20 mM at Tris pH 7 and 20 mM Tris at pH 8 with 125 mM NaCl, respectively. The elution buffer was 1 M NaCl for both. PCR analysis of the flowthrough and wash samples indicated that the lentiviral vectors were completely retained on the anion exchange columns. However, no elution was observed for these columns.

FIG. 18 shows the results of static bind and-elute test using 0.45 μm and 1.0 μm pore size Con A separation media discs (bed volume was 0.014 mL) and a 1 μm pore size blank media (a control). 1 μm pore size polymer grafted separation media having separation ligands including boronate affinity groups or multimodal cation-exchange TSA ligands were also tested. TSA may also have heparin mimicking properties. The separation media were incubated in lentiviral vector supernatant (1 mL, 1.42×10⁶ viral particles/mL) for 2 h. The Con A and blank media were washed with 20 Tris at pH 7.4, 0.5 M NaCl and eluted with 1 M methylmannoside. The boronate affinity groups and TSA ligands containing separation media were washed with 1×PBS pH 7.3 and eluted with 1 M sorbitol and 1 M NaCl, respectively. Neither sample showed a high degree of lentiviral vector binding or elution.

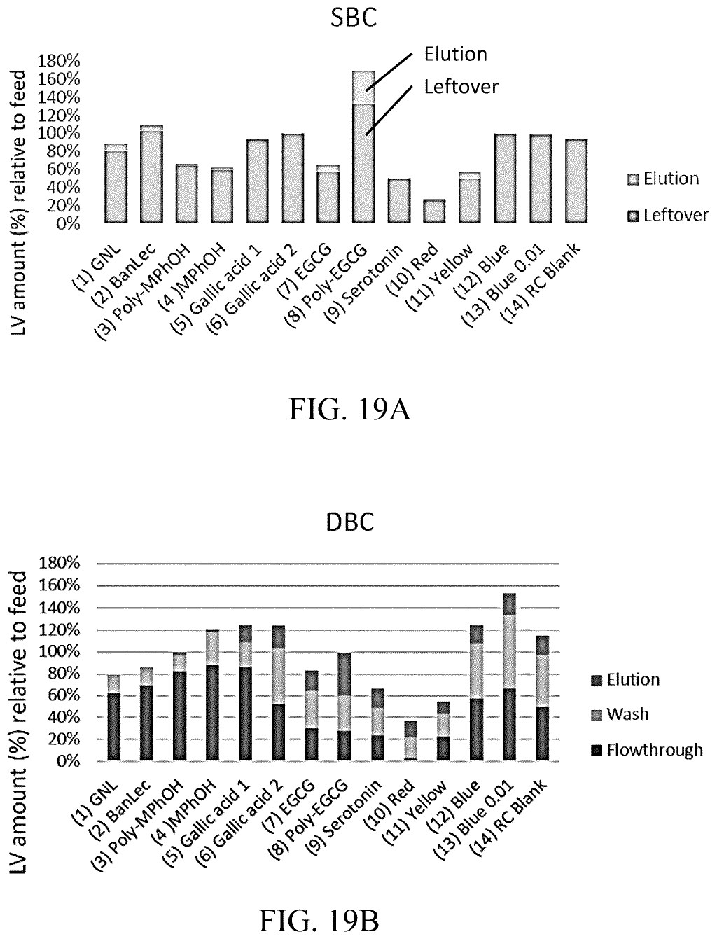

Next, a high throughput ligand screening study using both static and dynamic bind-and-elute experiments was performed. Two lectin ligands (Galanthus nivalis lectin (GNL) and Musa paradisiaca lectin (BanLec); carbohydrate binding domains) and eleven small molecule ligands where tested. Some of the separation media included the direct immobilization of the separation ligands and other included the indirect immobilization by polymer grafting (indicated by the prefix poly in FIGS. 19A and 19B). The average pore size was fixed at 1.0 μm, and a blank membrane sample was included for comparison. For the static testing, the membrane discs were incubated in 1 mL of lentiviral vector supernatant (1.91×10⁶ viral particles/mL). For dynamic testing, 2 mL of lentiviral vector supernatant (2.01×10⁶ viral particles/mL) was loaded onto the columns with a flow rate of 0.5 mL/min. The GNL and BanLec membranes were washed with 20 Tris at pH 7.4, 0.5 M NaCl and eluted with 1 M methylmannoside. All the other samples were washed with 1×PBS at pH 7.3 and eluted with 1×PBS at pH 7.3 with 1 M NaCl. The static and dynamic test results are shown in FIG. 19A and FIG. 19B, respectively. The most promising ligands identified in this study were EGCG (samples 7 and 8, with and without polymer grafting), and red dye (sample 10). The EGCG sample that included polymer grafting had the highest elution recovery for both static and dynamic testing, while the red dye affinity sample exhibited the highest binding capacity.

### Example 7: Example Processes Employing Immobilized Ligands for Capture of Fetuin and Asialofetuin Targets

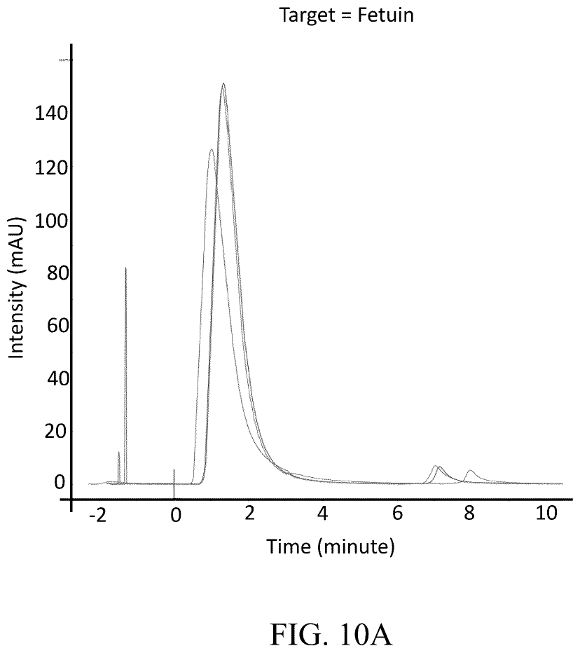

A separation media was prepared according to FIG. 9 (see Example 1) by immobilizing a lectin that can bind to 2,3 sialic acid or a lectin that can bind to 2,6 sialic acid (SIAFIND alpha 2,6-specific and SIAFIND alpha 2,3-specific; available from Lectenz Bio in Athens, GA). Both separation medias was tested by exposure to an isolation solution that included either fetuin or asialofetuin as a target. Specifically, a separation membrane having a 0.075 mL bed volume was challenged with triplicate bind and elute cycles using 0.5 mL of a 50 mM 4-(2-hydroxyethyl)piperazine-1-propanesulfonic acid (EPPS) with either 0 mM, 250 mM, or 500 mM NaCl containing 4 mg/mL fetuin or asialofetuin at a flow rate of 1 mL/min of the same buffer. Elutions were all performed with 50 mM EPPS with 1 M NaCl at the same flow rate. The lectin's activity toward 2,3 and 2,6 sialic acids is differentially modulated by NaCl concentration. Asialofetuin is partially desialyated fetuin and has lesser sialic acids content. FIG. 10A and FIG. 10B are example chromatograms of the binding experiment. Each trace is one of the triplicate runs.

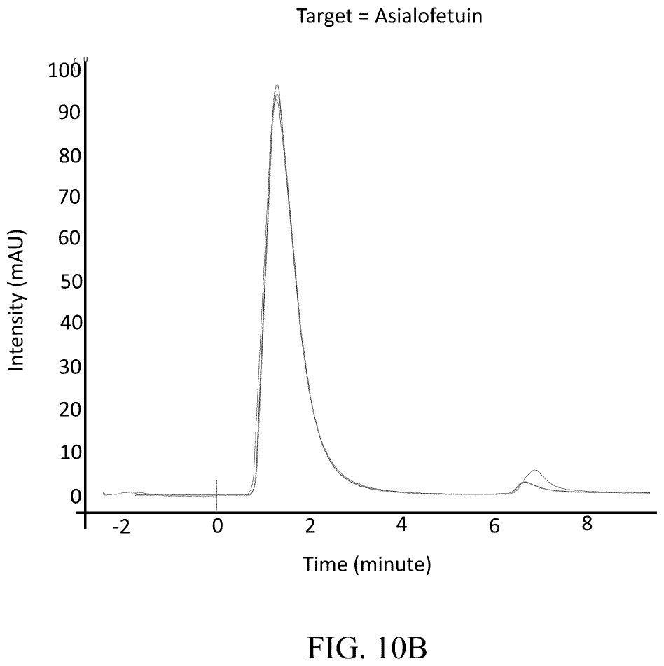

FIG. 11 summarizes membrane testing of processes using 50 mM EPPS with variable NaCl content. There was higher elution mass with fetuin relative to asialofetuin at 50 mM EPPS with 0 mM NaCl (32% reduction of elution mass with asialofetuin). Increasing the salt content 50 mM NaCl reduced performance ˜2.9 fold for fetuin compared to the same process with 0 mM NaCl for asialofetuin. Increasing salt to 100 mM further reduces asialofetuin elution masses by 3%. This is a much greater reduction of binding with less additional NaCl with asialofetuin compared to fetuin; fetuin elution masses were 66% of the same process at 0 NaCl at 250 mM NaCl compared to the lesser, 23%, elution mass of asialofetuin using a process with 50 mM EPPS with 100 mM NaCl. Processes using 50 mM EPPS with 500 mM NaCl exhibit over 50 times the elution mass compared to asialofetuin; at this concentration elution masses of fetuin are 53% of the elution masses of processes at 0 mM NaCl while elution masses of asialofetuin are 1% of the mass of fetuin eluted from a process using 50 mM EPPS with 0 mM NaCl. Overall this indicates a membrane chromatographic process with specificity towards sialic acid groups.

### Example 8: Example Processes Employing Immobilized Ligands for Capture of Sugar Recognizant Targets

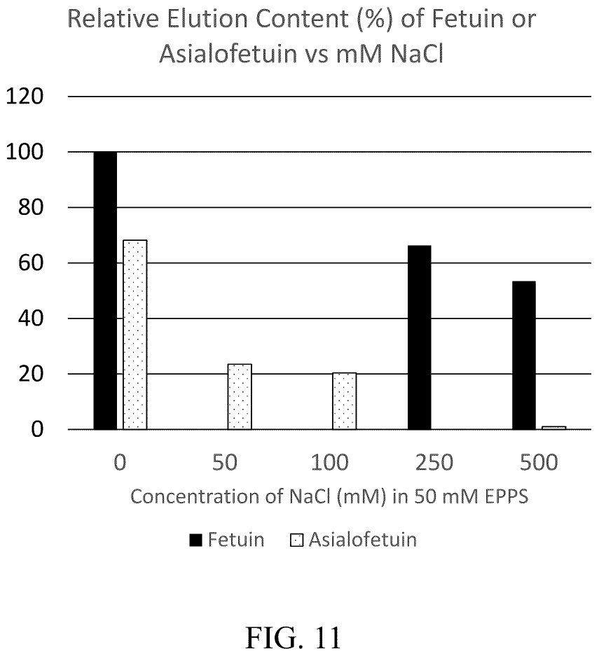

In a reciprocal fashion to Example 7, sialic acid rich ligands fraction IV fetuin (a fetuin derived from fraction iv of plasmid), fetuin derived from fetal bovine serum (Purilogics fetuin in FIG. 18), or alpha2-HS-glycoprotein (human fetuin A) were immobilized using the methodology in FIG. 9 (see Example 1) to exemplify a process in which targets that exhibit glycospecificity as the binding target. Membranes with immobilized ligands were challenged with the lectins discussed in Example 6 (SIAFIND alpha 2,6-specific and SIAFIND alpha 2,3-specific; available from Lectenz Bio in Athens, GA), which have activity toward 2,3 or 2,6 sialic acids with differing affinities. Static binding capacities (SBC) for the lectin with 2,3 sialic acid specificity were determined by incubating 0.0055 mL membrane in 0.5 mL of 1 mg/mL of a lectin having 2,3 sialic acid specificity in 50 mM EPPS 100 mM NaCl with for 2 hours. Static binding capacities for lectin having 2,6 sialic acid specificity were determined by incubating 0.0055 mL membrane in 0.5 mL of 0.2 mg/mL of lectin having 2,3 sialic acid specificity in 50 mM EPPS 100 mM NaCl with for 2 hours. Static binding performance is indicated in FIG. 12. The differential proportion in 2,3 vs 2,6 performance for each type of membrane indicates linkage specific affinity that can be leveraged in chromatographic separations.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1.-28. (canceled)

29. A method of isolating a target molecule from an isolation solution,
the isolation solution comprising:
an isolation solvent; and
the target molecule comprising a free protein and the free protein comprising a carbohydrate,
the method comprising:
contacting the isolation solution with a separation media,
the separation media comprising:
a support substrate; and
a plurality of separation ligands for formula SL1 or SL2 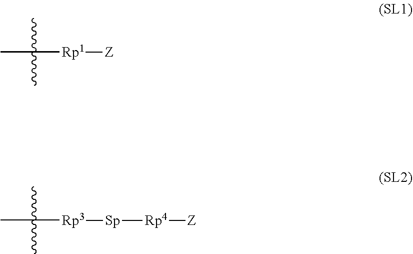
wherein:
Z is a separation group comprising an affinity group, the affinity group comprising a carbohydrate binding domain, a carbohydrate binging ligand, or both; and
Rp¹, Rp³, and Rp⁴ each independently comprise the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, Rp^{L}, and Rp^{M} or an isomer thereof, wherein Rp^{A}, Rp^{B}, Rp^{C}, Rp^{D}, Rp^{E}, Rp^{F}, Rp^{G}, Rp^{H}, Rp^{I}, Rp^{J}, Rp^{K}, Rp^{L}, and Rp^{M} are represented by: 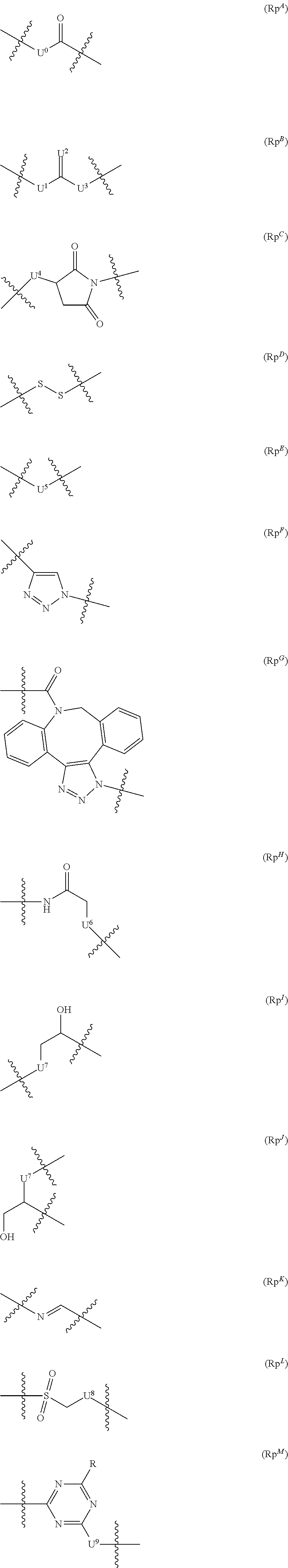
wherein U⁰, U¹, U², U³, U⁴, U⁵, U⁶, U⁷, U⁸, and U⁹ are each independently NH, O, or S;
R is an organic group, H, or halogen; and
Sp is a spacer comprising a divalent organic group.

30. The method of claim 29, wherein the alkanediyl or alkenediyl comprises a backbone chain of length C1 to C3.

31. The method of claim 29, wherein Sp comprising —C(O)—.

32. The method of claim 29, wherein Rp³, Rp⁴, or both comprises Rp^{E}.

33. The method of claim 32, wherein each U⁵ is O, each U⁵ is NH, or one U⁵ is NH and one U⁵ is O.

34. The method of claim 29, wherein SL2 comprises

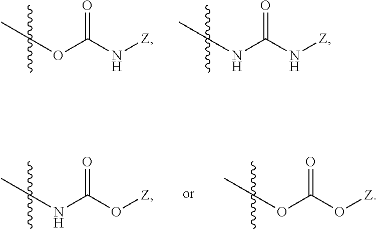

35. The method of claim 29, wherein SL2 comprises 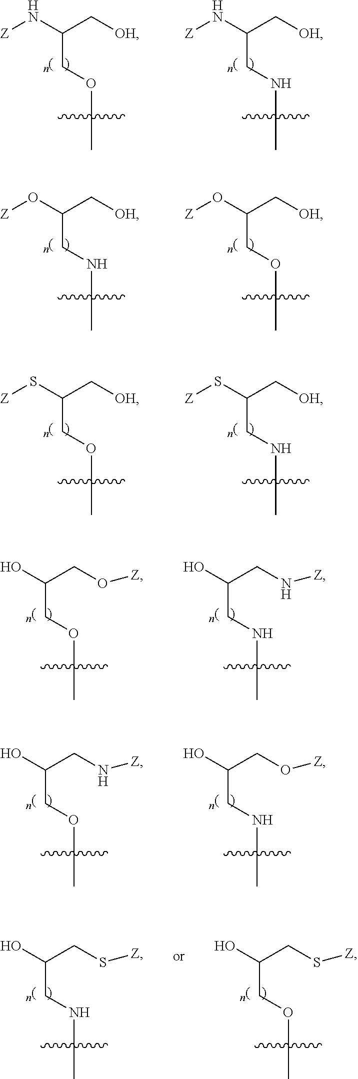
wherein n is 0, 1, 2, 3, 4, 5, 6, 7, 8, 9, or 10.

36. The method of claim 35, wherein n is 1.

37. The method of claim 29, wherein the support substrate comprises a polyolefin membrane, a polyethersulfone membrane, a poly(tetrafluoroethylene) membrane, a nylon membrane, a fiberglass membrane, a hydrogel membrane, a hydrogel monolith, a polyvinyl alcohol membrane, a cellulose membrane, a cellulose ester membrane, a cellulose acetate membrane, a regenerated cellulose membrane, a cellulosic nanofiber membrane, a cellulosic monolith, a filter paper, or any combination thereof.

38. The method of claim 29, wherein the method further comprises washing the separation media with a washing solution.

39. The method of any claim 29, further comprises eluting the target molecule from the separation media with an elution solution.

40. The method of claim 39 wherein the elution solution comprises an affinity group competitive molecule.

41. The method of claim 29, wherein the carbohydrate of the target molecule comprises sialic acid.

42. The method of claim 41, wherein the sialic acid is α2,6 linked sialic acid.

43. The method of claim 41, wherein the sialic acid is α2,3 linked sialic acid.

44. The method of claim 29, wherein the carbohydrate of the target molecule comprises fucose.

45. The method of claim 29, wherein the target molecule comprises an antibody or a fragment thereof.

46. The method of claim 29, wherein the target molecule comprises a first carbohydrate and a second carbohydrate.

47. The method of claim 46, wherein the first carbohydrate and the second carbohydrate are different.

48. The method of claim 47, wherein the first carbohydrate comprises α2,6 linked sialic acid and the second carbohydrate comprises α2,3 linked sialic acid.

49. The method of claim 29, wherein the target molecule comprises fetuin or a fragment thereof.
